(19) United States
(12) Patent Application Publication
(10) Pub. No.: US 2025/0384967 A1
(43) Pub. Date: Dec. 18, 2025

(54) Systems, Methods, Non-Transitory Instructions, and Apparatuses for Implementing a Workflow

(71) Applicant: DeepCure Inc., Boston, MA (US)

(72) Inventors: Chris MacNaughton, Fairland, IN (US); Heather Osswald, San Diego, CA (US); Luke Nam, Cambridge, MA (US); Derrick Miyao, San Diego, CA (US); Thrasyvoulos Karydis, Boston, MA (US)

(21) Appl. No.: 19/229,981

(22) Filed: Jun. 5, 2025

Related U.S. Application Data
(60) Provisional application No. 63/660,363, filed on Jun. 14, 2024.

(51) Int. Cl. G16C 20/10; G05B 15/02

(52) U.S. Cl. CPC G16C 20/10; G05B 15/02

(57) ABSTRACT

Systems and methods for implementing a workflow are provided. A workflow is obtained including a plurality of target compounds and a plurality of synthesis tasks collectively configured to synthesize the plurality of target compounds. Each synthesis task includes a corresponding specification including an identification of respective solvent, an amount of a reactant, an address of a well in a multi-well plate, a reaction duration, a reaction temperature, and a reaction volume. A respective subset of the plurality of synthesis tasks is performed including reacting the plurality of reactants in the plurality of wells in accordance with the corresponding specification of at least the respective subset of the plurality of synthesis tasks. A data set associated with a physical property of a liquid in each well specified by the corresponding specification is informed. The performing and obtaining are repeated until each synthesis task has been performed. An amount of each target compound that was synthesized is determined and used to amend the corresponding specification of each synthesis task in the plurality of synthesis tasks.

### CROSS REFERENCE TO RELATED APPLICATIONS

This application claims priority to U.S. Provisional Patent Application No. 63/660,363, entitled “SYSTEMS, METHODS, NON-TRANSITORY INSTRUCTIONS, AND APPARATUSES FOR IMPLEMENTING A WORKFLOW,” filed Jun. 14, 2024, which is hereby incorporated by reference.

### TECHNICAL FIELD

The present application is directed to implementing a workflow for generating compounds from synthons.

### BACKGROUND

Pharmaceutical companies spend millions of dollars screening compounds to discover novel compounds and develop them into prospective drug leads. Traditionally, this has involved collecting and testing large libraries of compounds to find a small number of compounds that interact with the disease target of interest. Unfortunately, the cost and time needed to physically assay compounds is prohibitive to testing them at scale.

Despite decades of effort and millions of dollars spent on end-to-end automation, drug discovery is conventionally driven by manual lab processes. End-to-end automated platforms have largely fallen short of expectations because traditional automation relies on worklists designed around single, fixed-input processes. These traditional worklists are unsuitable for driving complex, multi-instrument workflows with dynamically changing parameters. Further, traditional worklists require manual customization for each iteration of the design-make-test cycle.

Given the above background, what is needed in the art are improved methods for designing, developing, and/or synthesizing compounds for drug discovery.

### SUMMARY

The present disclosure addresses the problems identified in the background by providing systems and methods that make use of automated reaction devices, machine learning models, workflows, and/or pipelines thereof to facilitate development, synthesis, optimization, and/or screening of compounds for drug discovery. In particular, the disclosed systems and methods utilize a framework for dynamic performance of molecular reactions to enable automation of such processes. In some embodiments, the framework includes the generation, optimization, and/or selection of various elements involved in such processes. Furthermore, in some embodiments, the framework further contemplates molecular reaction conditions, instances of molecular reactions (e.g., reaction wells), synthons, and/or molecular products, as well as model inputs or outputs comprising the same. Advantageously, in some implementations, the disclosed systems and methods allow a platform for one or more of compound development, synthesis, and screening. Moreover, in some implementations, the disclosed systems and methods are agnostic to the type of automated workflow used and removes the need for scientists to review outputs between stages of execution. In some implementations, the disclosed systems and methods also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and compound testing for drug discovery using machine learning models.

Accordingly, one aspect of the present disclosure provides a method for implementing a workflow. The method includes obtaining an initial workflow. The initial workflow includes a selection of a plurality of target compounds and a plurality of synthesis tasks collectively configured to synthesize each target compound in the plurality of target compounds. Each synthesis task in the plurality of synthesis tasks includes a corresponding specification including an identification of respective solvent in one or more solvents, an amount of at least one reactant in a plurality of reactants, an x-y address of a well in a first multi-well plate including a plurality of wells, a reaction duration, a reaction temperature, and a reaction volume. The method further includes performing at least a respective subset of the plurality of synthesis tasks of the initial workflow at a molecular foundry. The molecular foundry includes a plate handler for the first multi-well plate and one or more liquid handlers for at least the plurality of reactants. The performing includes reacting the plurality of reactants in the plurality of wells in accordance with the corresponding specification of at least the respective subset of the plurality of synthesis tasks. Additionally, the method includes informing a first data set associated with a physical property of a liquid in each well specified by the corresponding specification of each synthesis task in at least the respective subset set of synthesis tasks. Furthermore, the method includes repeating the performing the at least a respective subset of the plurality of synthesis tasks and obtaining until each synthesis task in the plurality of synthesis tasks has been performed. Moreover, the method includes determining an amount of each target compound in the plurality of target compounds that was synthesized in accordance with the plurality of synthesis tasks using the first data set. The method includes using the amount of each target compound in the plurality of target compounds that was synthesized to amend the corresponding specification of each synthesis task in the plurality of synthesis tasks.

In some embodiments, the using the amount of each target compound includes training a model that estimates target compound synthetic efficiency as a function of synthesis task specification. The model includes a plurality of parameters, and the model training adjusts these parameters in order to optimize model performance so that model accurately provides compound synthetic efficiency as a function of synthesis task specification. In some embodiments the model training is done through application of a procedure that includes: i) applying the synthesis task specification of one or more synthesis tasks in the plurality of synthesis tasks for the synthesis of a corresponding target compound in the plurality of target compounds thereby obtaining a calculated synthetic efficiency for the corresponding target compound; ii) determining a difference between (a) an efficiency of the corresponding target compound as determined by the model and (b) an actual (known) efficiency of the corresponding target compound as determined by the first data set; and iii) back-propagating the difference between the two through the model to adjust model parameters thereby training the model.

In some embodiments, the training procedure is repeated for each target compound, or batch of target compounds, in the plurality of target compounds.

In some embodiments, the plurality of synthesis tasks encodes a plurality of different specifications for synthesizing a first target compound in the plurality of target compounds. Moreover, in some such embodiments, the using the amount of each target compound includes pruning out a first subset of the plurality of different specifications for synthesizing the first target compound from the initial synthesis tasks that the first data set indicates synthesized the first target compound at a lower efficiency than a second subset of the plurality of different specifications for synthesizing the first target compound.

In some embodiments, the physical property is determined using spectroscopy.

In some embodiments, the spectroscopy is ultraviolet (UV) spectroscopy and the physical property is absorbance of UV light.

In some embodiments, the spectroscopy is light spectroscopy and the physical property is absorbance of visible light.

In some embodiments, the spectroscopy is infrared (IR) spectroscopy and the physical property is absorbance of IR light.

In some embodiments, the spectroscopy is atomic absorption spectroscopy and the physical property is absorbance of light.

In some embodiments, the spectroscopy is inductively coupled plasma optical emission spectroscopy (ICP-OES) and the physical property is light emission.

In some embodiments, the spectroscopy is fluorescence spectroscopy and the physical property is light emission.

In some embodiments, the spectroscopy is Raman spectroscopy and the physical property is vibrational or rotational model of atoms of the target compound.

In some embodiments, the initial workflow further includes one or more plating tasks, one or more filtration tasks, one or more dilution tasks, one or more analytical tasks, or any combination thereof.

In some embodiments, the plurality of target compounds consists of organic compounds.

In some embodiments, the performing the at least a respective subset of the plurality of synthesis tasks and/or the informing the first data set associated with the physical property of a liquid is conducted without human intervention.

In some embodiments, the performing the at least a respective subset of the plurality of synthesis tasks further includes illuminating a field of view across the first multi-well plate with substantially uniform optical characteristics across the field of view.

In some embodiments, the spectral range of light when illuminating the field of view is between 250 nanometers (nm) and 315 nm.

In some embodiments, the first data set includes a first plurality of data elements associated with the field of view prior to the illuminating and a second plurality of data elements associated with the field of view when illuminated during the informing the first data set associated with the physical property.

In some embodiments, the first data set includes a first plurality of data elements associated with one or more reaction conversion rates when producing at least one target compounds in the plurality of target compounds.

In some embodiments, the first data set includes at least one corresponding alphanumeric identifier for each target compound in the plurality of target compounds that identifies a well in the multi-well plate that the initial workflow specifies contains the target compound.

In some embodiments, the first data set includes at least one corresponding set of Cartesian coordinates for each target compound in the plurality of target compounds that identifies a well in the multi-well plate that the initial workflow specifies contains the target compound.

In some embodiments, the first data set includes one or more spatial coordinates associated for each target compound in the plurality of target compounds that identifies a well in the multi-well plate that the initial workflow specifies contains the target compound, a temporal identifier of an amount of time in the corresponding specification of a synthesis task associated with the respective target compound, a spectral identifier of one or more wavelengths associated with the respective target compound, or a combination thereof.

In some embodiments, the multi-well plate includes between 24 and 384 wells.

In some embodiments, the reaction duration is between 1 second and two days.

In some embodiments, the reaction temperature is between 0° C. and 99° C.

In some embodiments, at least two synthesis tasks in the plurality of synthesis tasks are required to synthesize a first compound in the plurality of compounds and the performing the at least a respective subset of the plurality of synthesis tasks and informing the first data set associated with the physical property is performed a first time for a first synthesis task in the at least synthesis two tasks and the performing and informing the first data set associated with the physical property is performed a second time, after the first synthesis task, for a second synthesis task in the at least two synthesis tasks.

In some embodiments, the respective solvent in a corresponding specification is water, dimethyl sulfoxide (DMSO), acetonitrile, dimethyl ether, chloroform, hexanes, toluene, dichloromethane, N-Methyl-2-pyrrolidone (NMP), methanol, or a combination thereof.

In some embodiments, the reaction volume is between 10 μL and 10000 μL.

In some embodiments, the plurality of target compounds includes 10 or more compounds. In some embodiments, the plurality of target compounds includes 100 or more compounds. In some embodiments, the plurality of target compounds includes 1000 or more compounds.

In some embodiments, the plurality of synthesis tasks includes 20 or more synthesis tasks. In some embodiments, the plurality of synthesis tasks includes 50 or more synthesis tasks. In some embodiments, the plurality of synthesis tasks includes 100 or more synthesis tasks. In some embodiments, the plurality of synthesis tasks includes 1000 or more synthesis tasks.

In some embodiments, a first synthesis task in the plurality of synthesis task includes: a first plurality of step instructions for controlling a first pipettor in fluid communication with a first reactant, in the one or more reactants specified by the first synthesis task, that is dissolved in a respective solvent specified by the first synthesis task, and a plurality of x-y plate instructions for causing the x-y address of a first well in the first multi-well plate specified by the first synthesis task to be in fluid communication with the first pipettor.

In some embodiments, the first synthesis task further includes: a set of instructions to cause the first pipettor to switch from being in fluid communication with the first reactant, to being in fluid communication with a second reactant, in the one or more reactants specified by the first synthesis task, that is dissolved in a respective solvent specified by the first synthesis task, and a plurality of step instructions for causing the first pipettor to dispense an amount of the second reactant that is dissolved in a respective solvent, specified by the first synthesis task specified by the first synthesis task, into the first well.

In some embodiments, each target compound in the plurality of target compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, each target compound in the target plurality of target compounds is organic compound having a molecular weight of less than 500 Daltons. In some embodiments, each target compound in the plurality of target compounds is an organic compound having a molecular weight of less than 1000 Daltons. In some embodiments, each target compound in the plurality of target compounds is an organic compound having a molecular weight of less than 2000 Daltons.

In some embodiments, each target compound in the plurality of target compounds is an organic compound having a molecular weight of less than 4000 Daltons, less than 6000 Daltons, less than 8000 Daltons, less than 10000 Daltons, or less than 20000 Daltons.

In some embodiments, each target compound in the plurality of target compounds is organic compound having a molecular weight of between 300 Daltons and 1500 Daltons.

In some embodiments, a first synthesis task in the plurality of synthesis tasks specifies an amount of a first reactant as a number of equivalents relative a second reactant specified by the first synthesis task to be added to the well specified by the first synthesis task.

In some embodiments, a first synthesis task in the plurality of synthesis tasks specifies an amount of a first reactant as a number of moles of the first reactant to be added to the well specified by the first synthesis task.

In some embodiments, a first synthesis task in the plurality of synthesis tasks specifies an amount of a first reactant as a mass of the first reactant to be added to the well specified by the first synthesis task.

In some embodiments, the corresponding specification of the synthesis task further includes a reaction task.

In some embodiments, the reaction task is an agitation task, a mixing task, a liquid chromatography task, or a temperature gradient task.

Another aspect of the present disclosure is directed to providing a computer system for implementing a workflow. The computer system includes one or more processors, and a memory coupled to the one or more processors. The memory includes one or more programs configured to be executed by the one or more processors, which causes the computer system to perform a method of the present disclosure.

Another aspect of the present disclosure is directed to providing a non-transitory computer readable storage medium storing one or more programs. The one or more programs includes instructions, which when executed by a computer system cause the computer system to perform a method of the present disclosure.

Another aspect of the present disclosure is directed to providing a device for implementing a workflow. The device includes one or more processors and a memory coupled to the one or more processors, the memory including one or more programs configured to be executed by the one or more processors, thereby causing the device to perform a method of the present disclosure.

Yet another aspect of the present disclosure provides a method for visualizing reaction conversion. The method includes obtaining a selection of a first multi-well plate in a plurality of multi-well plates, in which each multi-well plate in the plurality of multi-well plates includes an array of wells. The method further includes assigning a corresponding identifier to each respective well of the first multi-well plate. The corresponding identifier is associated with both a corresponding reagent-solvent pairing accommodated by the respective well and a corresponding reaction stoichiometry. Additionally, the method includes evaluating, when performing a reaction at a molecular foundry, a conversion for each respective well of the first multi-well plate, which produces a conversion data set. Moreover, the method includes generating, for display through a graphical user interface, a visualization of the first multi-well plate based on the conversion data set, thereby visualizing the reaction conversion.

In some embodiments, the selection of the first multi-well plate defines two or more dimensions of the first multi-well plate.

In some embodiments, each respective well in the array of wells is uniquely defined by a different reagent, a different solvent, a different reaction stoichiometry, or a combination thereof.

In some embodiments, the corresponding reaction stoichiometry defines a reagent:solvent ratio.

In some embodiments, the visualization includes a local heat map associated with a respective conversion rate for each corresponding well in the array of wells of the first multi-well plate.

In some embodiments, the local heat map includes a color gradient mapping for each corresponding well in the array of wells of the first multi-well plate.

In some embodiments, the color gradient mapping is based on a linear scale, a logarithmic scale, a rank scale, or an exponential scale.

In some embodiments, in accordance with a determination, for each respective well in the array of wells of the first multi-well plate, that the conversion exceeds a first threshold conversion rate, a first color of the color gradient mapping is assigned to the respective well, and in accordance with a determination, for each respective well in the array of wells of the first multi-well plate, that the conversion fails to exceed the first threshold conversion rate, a second color of the color gradient mapping is assigned to the respective well.

In some embodiments, in accordance with a determination, for each respective well in the array of wells of the first well plate, that the conversion exceeds a first threshold conversion rate, a first color of the color gradient mapping is assigned to the respective well, and in accordance with a determination, for each respective well in the array of wells of the first multi-well plate, that the conversion exceeds a second threshold conversion rate, a second color of the color gradient mapping is assigned to the respective well, and in accordance with a determination, for each respective well in the array of wells of the first multi-well plate, that the conversion exceeds the first threshold conversion rate but not the second threshold conversion rate, a third color of the color gradient mapping is assigned to the respective well.

In some embodiments, the local heat map includes an indicia mapping for each corresponding well in the array of wells of the first multi-well plate.

In some embodiments, in accordance with a determination, for each respective well in the array of wells of the first multi-well plate, that the conversion exceeds a third threshold conversion rate, a first indicia is assigned to the respective well, and in accordance with a determination, for each respective well in the array of wells of the first multi-well plate, that the conversion fails to exceed the third threshold conversion rate, no indicia is assigned to the respective well.

In some embodiments, the visualization includes a regional heat map associated with a respective conversion for a subset of wells in the array of wells of the first multi-well plate.

In some embodiments, each well in the subset of wells shares a contiguous boundary with at least one well in the subset of wells.

In some embodiments, an exterior boundary of the subset of wells forms a closed-form polygon.

In some embodiments, the regional heat map includes an opacity gradient mapping for each corresponding well in the array of wells of the first multi-well plate.

In some embodiments, the opacity gradient mapping is based on a linear scale, a logarithmic scale, a rank scale, or an exponential scale.

In some embodiments, in accordance with a determination of a collective conversion of the subset of wells exceeds a fourth threshold conversion rate, a first opacity of the opacity gradient mapping is assigned to the subset of wells, and in accordance with a determination a collective conversion of the subset of wells fails to exceed the fourth threshold conversion rate, a second opacity of the opacity gradient mapping is assigned to the subset of wells.

Yet another aspect of the present disclosure provides computer system for visualizing reaction conversion. The computer system includes one or more processors, and a memory coupled to the one or more processors. The memory includes one or more programs configured to be executed by the one or more processors, thereby causing the computer system to perform a method of the present disclosure.

Yet another aspect of the present disclosure provides a non-transitory computer readable storage medium storing one or more programs, the one or more programs including instructions, which when executed by a computer system cause the computer system to perform a method of the present disclosure.

Yet another aspect of the present disclosure provides a device for visualizing reaction conversion, the device including one or more processors and a memory coupled to the one or more processors, the memory including one or more programs configured to be executed by the one or more processors, thereby causing the device to perform a method of the present disclosure.

Yet another aspect of the present disclosure provides a method for performing a synthesis and/or purification task at a molecular foundry. The method includes obtaining a first workflow including a plurality of tasks for performing a first reaction. The plurality of tasks includes a liquid chromatography task, an evaporation task, an agitation task, or a combination thereof. The method further includes assigning a first selection of parameters for each task in the plurality of tasks. Moreover, the method includes executing the first workflow using the first selection of parameters. The method further includes determining, concurrent with the executing, (i) a conversion efficiency of the first reaction in making a target compound and/or (ii) a purity of the target compound. Furthermore, the method includes assigning a second selection of parameters for one or more tasks in the plurality of tasks. The second selection of parameters includes an increase in at least one parameter in the first selection of parameters in accordance with a determination that the conversion efficiency of the first reaction and/or the purity of the target compound fails to satisfy a threshold conversion efficiency or purity, and the second selection of parameters includes a decrease in at least one parameter in the first selection of parameters in accordance with a determination that the conversion efficiency of the first reaction or the purity of the target compound satisfies the threshold conversion efficiency or purity. Additionally, the method includes generating a second workflow including some or all of the plurality of tasks for performing the first reaction, wherein the second workflow includes the second selection of parameter. The method further includes executing the second workflow, thereby performing the synthesis and/or purification task at the molecular foundry.

In some embodiments, the plurality of tasks includes the liquid chromatography task. The liquid chromatography task includes one or more column location parameters, one or more solvent parameters, one or more mobile phase gradient parameters, one or more mass flow rate parameters, one or more stoichiometry parameters, one or more wavelength parameters, one or more epoch parameters, one or more volumetric parameters, one or more resolution parameters, one or more mass parameters, or a combination thereof.

In some embodiments, the plurality of tasks includes the evaporation task and the evaporation task includes one or more epoch parameters, one or more velocity parameters, one or more stoichiometry parameters, one or more temperature parameters, one or more frequency parameters, one or more pressure parameters, one or more human interaction parameters, or a combination thereof.

In some embodiments, the plurality of tasks includes the agitation task and the agitation task includes one or more temperature parameters, one or more power source parameters, one or more frequency parameters, one or more amplitude parameters, one or more epoch parameters, or a combination thereof.

In some embodiments, the first selection of parameters includes an amount of agitation.

In some embodiments, the first selection of parameters includes an amount of a reactant.

In some embodiments, the first selection of parameters includes a reaction duration.

In some embodiments, the first selection of parameters includes a reaction temperature.

In some embodiments, the first selection of parameters includes a stoichiometric ratio between a first reactant and a second reactant.

In some embodiments, the first selection of parameters includes a stoichiometric ratio between a first reactant and a second reactant.

In some embodiments, the first selection of parameters includes a reaction temperature.

In some embodiments, the threshold conversion efficiency or purity is a threshold conversion efficiency.

In some embodiments, the conversion efficiency is between 20 percent and 80 percent.

In some embodiments, the threshold conversion efficiency or purity is a threshold purity.

In some embodiments, the purity is between 20 percent and 80 percent.

Another aspect of the present disclosure provides a computer system for performing a synthesis and/or purification task. The computer system includes one or more processors and a memory coupled to the one or more processors. The memory includes one or more programs configured to be executed by the one or more processors, thereby causing the computer system to perform a method of the present disclosure.

Another aspect of the present disclosure provides a non-transitory computer readable storage medium storing one or more programs, the one or more programs including instructions, which when executed by a computer system cause the computer system to perform a method of the present disclosure.

Another aspect of the present disclosure provides a device for performing a synthesis and/or purification task. The device includes one or more processors and a memory coupled to the one or more processors. The memory includes one or more programs configured to be executed by the one or more processors, thereby causing the device to perform a method of the present disclosure.

Yet another aspect of the present disclosure provides methods for visualizing reaction conversions. The methods include obtaining a selection of a first multi-well plate in a plurality of multi-well plates. Each multi-well plate in the plurality of multi-well plates includes an array of wells. The methods further include assigning a corresponding identifier to each respective well of the first multi-well plate. Additionally, the methods include obtaining a plurality of user-defined conditions and a plurality of geometric conditions for each respective well of the first multi-well plate. Furthermore, the methods include generating, for display through a graphical user interface, a visualization of the first multi-well plate based on the corresponding identifier assigned to each respective well in the array of wells, the plurality of user-defined conditions, and the plurality of geometric conditions, thereby providing visualization of the reaction conversions.

In some embodiments, the corresponding identifier includes an alphanumeric identifier associated with a first position in the array of wells.

In some embodiments, the corresponding identifier includes a Cartesian identifier associated with a second position in the array of wells.

In some embodiments, the plurality of user-defined conditions includes one or more local reaction conditions associated with a first well in the array of wells of the first multi-well plate.

In some embodiments, the plurality of user-defined conditions includes one or more regional reaction conditions associated with a subset of wells in the array of wells of the first multi-well plate.

In some embodiments, each well in the subset of wells shares a contiguous boundary with at least one well in the subset of wells.

In some embodiments, the subset of wells includes a second well that lacks a contiguous boundary with at least one well in the subset of wells.

In some embodiments, the one or more regional reaction conditions includes a reagent-solvent pairing condition.

In some embodiments, the one or more regional reaction conditions includes a reaction stoichiometry condition between a first reactant and a second reactant.

In some embodiments, the plurality of user-defined conditions includes one or more global reaction conditions associated with each well in the array of wells of the first multi-well plate.

In some embodiments, the one or more global reaction conditions includes one or more epoch conditions, one or more temperature conditions, or a combination thereof.

In some embodiments, the plurality of user-defined conditions includes one or more reactants in one or more wells in the array of wells of the first multi-well plate.

In some embodiments, the plurality of user-defined conditions defines a first order of operations associated with the first multi-well plate, a second order of operations associated with the subset of wells, a third order of operations associated with each well in the array of multi-wells, or a combination thereof.

In some embodiments, the plurality of user-defined conditions defines one or more Boolean conditions for visualizing a status of a respective solution of one or more wells in the array of wells.

In some embodiments, in accordance with a determination a first well in the array of wells satisfies a first Boolean condition, assigning a first color to the first well, and

in accordance with a determination the first well in the array of wells fails to satisfy the first Boolean condition, assigning a second color to the first well.

In some embodiments, the plurality of geometric conditions includes one or more dimensions associated with each respective well in the array of wells.

Another aspect of the present disclosure provides a computer system for visualizing reaction conversion. The computer system includes one or more processors and a memory coupled to the one or more processors. The memory includes one or more programs configured to be executed by the one or more processors, thereby causing the computer system to perform a method of the present disclosure.

Another aspect of the present disclosure provides a non-transitory computer readable storage medium storing one or more programs. The one or more programs include instructions, which when executed by a computer system cause the computer system to perform a method of the present disclosure.

Another aspect of the present disclosure provides a device for visualizing reaction conversions. The device includes one or more processors and a memory coupled to the one or more processors. The memory includes one or more programs configured to be executed by the one or more processors, thereby causing the device to perform a method of the present disclosure.

Yet another aspect of the present disclosure provides a method for drawing a solution into a robotic pipette tip. The method includes obtaining a status of a solution in a first well in a multi-well container. The status of the solution includes an identification of the solution and at least one dimension of the first well. In some embodiments, the method includes obtaining, using a lookup table, a conductivity of the solution, which determines a volume of the solution accommodated by the first well based on the conductivity of the solution and the at least one dimension of the first well. In some embodiments, the method includes generating, in accordance with the volume of the solution and the at least one dimension of the first well, one or more execution instructions for drawing some or all of the solution from the first well using the robotic pipette tip including an opening at a lower end portion of the robotic pipette tip. In some embodiments, the one or more execution instructions includes at least one traverse instruction to traverse either the lower end portion of the robotic pipette tip or a lower end portion of the first well in a first vertical direction. The at least one traverse instruction includes a coordinate instruction and a velocity instruction. Furthermore, the method includes performing the one or more execution instructions at a liquid handling robot coupled to the robotic pipette tip, which draws the solution into the robotic pipette tip.

In some embodiments, the method further includes determining, concurrent with the performing the one or more execution instructions at a liquid handling robot, the solution capacitance. Moreover, in accordance with a determination the solution capacitance exceeds a first threshold value, the method includes continuing with the performing the one or more execution instructions at a liquid handling robot, and, in accordance with a determination the solution capacitance fails to exceed the first threshold value, ceasing the performing the one or more execution instructions at a liquid handling robot.

In some embodiments, identification of the solution includes a corresponding classification, in a plurality of classifications, of the solution.

In some embodiments, the plurality of classifications is defined, at least in part, by a first source. In some embodiments, the lookup table is obtained from the first source.

In some embodiments, the plurality of classifications includes a first classification associated with one or more solvents that exceeds a first threshold conductivity and a first threshold volatility.

In some embodiments, the one or more solvents of the first classification includes N-methyl-2-pyrrolidone (NMP), dimethyl sulfoxide (DMSO), water, acetic acid (AcOH), or a combination thereof.

In some embodiments, the plurality of classifications includes a second classification associated with one or more solvents that exceeds a second threshold conductivity and fails to exceed a second threshold volatility.

In some embodiments, the one or more solvents of the second classification includes methanol.

In some embodiments, the plurality of classifications includes a third classification associated with one or more solvents that fails to exceed a third threshold conductivity and exceeds a third threshold volatility.

In some embodiments, the plurality of classifications includes a fourth classification associated with one or more solvents that fails to exceed a fourth threshold conductivity and a fourth threshold volatility.

In some embodiments, the one or more solvents of the fourth classification includes N,N-diisopropylethylamine (DIPEA).

In some embodiments, the solution includes (i) a first component solution selected from the first classification or the second classification and (ii) a second component solution selected from the third classification or the fourth classification.

In some embodiments, the solution includes (i) a first component solution selected from the first classification or the second classification and (ii) a second component solution selected from the third classification or the fourth classification. In some embodiments, the solution exceeds the first threshold conductivity of the first classification or the second threshold conductivity of the second classification.

In some embodiments, the solution includes (i) a first component solution selected from the first classification or the second classification and (ii) a second component solution selected from the third classification or the fourth classification. In some embodiments, the solution exceeds the first threshold conductivity of the first classification and the second threshold conductivity of the second classification.

In some embodiments, the status of the solution of the first well further includes the depth of the solution.

In some embodiments, the status of the solution of the first well further includes the dead volume of the solution.

In some embodiments, the status of the solution of the first well further includes the depth of a lower end portion of the first well.

In some embodiments, the method further includes determining, concurrent with the performing the one or more execution instructions at the liquid handling robot, the depth of the lower end portion of the first well. In accordance with the depth of the lower end portion of the first well exceeding a second threshold value, continuing with the performing the one or more execution instructions at the liquid handling robot, and in accordance with the determination the depth of the lower end portion of the first well fails to exceed the second threshold value, ceasing the performing the one or more execution instructions at the liquid handling robot.

In some embodiments, the obtaining further includes using the lookup table to obtain the solution volatility.

In some embodiments, in accordance with a determination that the volume of the solution exceeds a third threshold value, the at least one traverse instruction includes traversing the lower end portion of the robotic pipette tip, and in accordance with a determination that the volume of the solution fails to exceed the third threshold value, the at least one traverse instruction includes traversing the lower end portion of the first well in the first vertical direction.

In some embodiments, the at least one dimension of the first well includes the interior volume of the first well.

In some embodiments, the obtaining further includes in accordance with a determination the volume of the solution satisfies a fourth threshold value based on the interior volume of the first well, conducting the generating, and in accordance with a determination the volume of the solution fails to exceed the fourth threshold value, ceasing the performing the one or more execution instructions at the liquid handling robot.

Yet another aspect of the present disclosure includes a system, including a memory; one or more processors; and one or more modules stored in the memory and configured for execution by the one or more processors, the one or more modules including instructions for performing any of the methods disclosed above.

Still another aspect of the present disclosure includes a non-transitory computer readable storage medium, the non-transitory computer readable storage medium storing one or more programs for execution by one or more processors of a computer system, the one or more computer programs including instructions for performing any of the methods disclosed above.

Another aspect of the present disclosure provides a device for drawing a liquid solution into a robotic pipette tip, the device including one or more processors and a memory coupled to the one or more processors, the memory including one or more programs configured to be executed by the one or more processors, thereby causing the device to perform any of the methods described above.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIG. 1 illustrates a system architecture in accordance with some embodiments of the present disclosure.

FIGS. 2A and 2B collectively illustrates various modules and components of a computing system in accordance with some embodiments of the present disclosure.

FIG. 2C illustrates various modules and components of a molecular foundry in accordance with some embodiments of the present disclosure.

FIGS. 3A, 3B, 3C, 3D, 3E, 3F, 3G, and 3H collectively illustrate a flowchart for implementing a workflow, in which optional steps are indicated by dashed lines, in accordance with some embodiments of the present disclosure.

FIGS. 4A, 4B, and 4C collectively illustrate a flowchart for visualizing reaction data including a reaction conversion, in which optional steps are indicated by dashed lines, in accordance with some embodiments of the present disclosure.

FIGS. 5A, 5B, and 5C collectively illustrate a flowchart for performing a synthesis and/or purification task at a molecular foundry, in which optional steps are indicated by dashed lines, in accordance with some embodiments of the present disclosure.

FIGS. 6A, 6B, and 6C collectively illustrate a flowchart for visualizing reaction data including a reaction conversion, in which optional steps are indicated by dashed lines, in accordance with some embodiments of the present disclosure.

FIGS. 7A, 7B, 7C, 7D, and 7E collectively illustrate a flowchart for drawing a liquid, in which optional steps are indicated by dashed lines, in accordance with some embodiments of the present disclosure.

FIGS. 8, 9, 10, 11, 12, and 13 illustrate a variety of user interfaces for visualizing reaction data including a reaction conversion in accordance with some embodiments of the present disclosure.

FIGS. 14, 15, 16, 17, and 18 illustrate a variety of user interfaces in accordance with some embodiments of the present disclosure.

FIGS. 19, 20, 21, 22, 23, 24, 25, 26, 27, and 28 illustrate a variety of user interfaces for implementing and/or designing a workflow in accordance with some embodiments of the present disclosure.

FIG. 29 illustrates a side view of a well of a multi-plate well and a pipettor drawing a liquid from the well in accordance with some embodiments of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The present disclosure addresses the problems identified in the background by providing systems and methods that make use of automated reaction devices, machine learning models, workflows, and/or pipelines thereof to facilitate development, synthesis, and/or screening of compounds for drug discovery. In particular, the disclosed systems and methods utilize a framework for dynamic performance of molecular reactions to enable automation of such processes. In some embodiments, the framework includes the generation, optimization, and/or selection of various elements involved in such processes. Furthermore, in some embodiments, the framework further contemplates molecular reaction conditions, instances of molecular reactions (e.g., reaction wells), synthons, and/or molecular products, as well as model inputs or outputs comprising the same.

Combining automation, chemistry, and machine learning can overcome human limitations in drug discovery. For instance, manual chemistry often leads to performing more of what an individual already knows. Typically, chemists approach drug design one parameter at a time, in addition to designing and synthesizing compounds one at a time. As such, the limitations of manual chemistry can impede the design of new molecules. Conversely, an automated chemical synthesis platform is as powerful as the reactions it can perform. More reactions is equivalent to more chemical space, which in turn enables machine learning tools to design and access a greater scope of multiparameter-designed molecules. Utilizing recent increases in computational power, an automated synthesis platform connected to compound screening and testing can enable standardized big data that have never before been possible. Such data can lead to improved models and designs of new molecules for drug discovery.

Advantageously, in some implementations, the disclosed systems and methods allow for target compound development, synthesis, and screening within a single platform (e.g., “design-make-test”). Moreover, in some implementations, the disclosed systems and methods are agnostic to the type of automated workflow used and remove the need for scientists to review outputs between stages of execution. In some implementations, the disclosed systems and methods also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and target compound testing for drug discovery using machine learning models.

In some embodiments, the use of machine learning models and/or automated reaction devices, such as an automated synthesis device or robot, improves the technical field of drug discovery.

Drug discovery efforts often suffer from significant bottlenecks, including the ability to identify hit compounds and validate any such identified hit compounds as lead compounds for downstream synthesis and testing. These difficulties can be attributed, at least in part, to the massive size of molecule libraries that are searched in these early stages, which can reach up to 10¹² candidate molecules. Conventional methods, including traditional screening and fragment-based screening require laborious hit identification and/or hit-to-lead steps that increase the overall time, cost, and resource expenditure of drug discovery.

In some embodiments, use of an automated reaction device improves the efficiency and speed of drug discovery and compound development processes by providing a mechanism for streamlined and dedicated preparation and implementation of molecular reactions, thereby relieving, at least in part, the bottlenecks described above. In contrast to manual processes, the automated reaction device reduces the amount of time, expertise, and human labor required to perform such reactions. In some embodiments, the automated reaction device further reduces human error, thereby increasing the accuracy and reliability of any generated experimental output. Similarly, in some embodiments, the automated reaction device further reduces variability due to human error or varying environmental conditions, thereby improving the reproducibility of the output.

In some embodiments, use of an automated reaction device further improves the efficiency of a computer-implemented method for drug discovery (e.g., for selection or optimization of reaction conditions and/or any synthons thereof), by reducing the bottleneck of human data collection, review, analysis, and input, in generating molecular outputs and/or updating or training a model to generate the same. Molecular outputs can include, for instance, molecular reactions, molecular products, reaction conditions, instances of molecular reactions, and/or synthons, among others.

In some embodiments, the systems and methods disclosed herein provide improvements to drug discovery and compound development by facilitating the use of machine learning models. In some embodiments, for instance, the training, development, and/or use of a machine learning model to predict various molecular outputs removes the need for laborious and exhaustive testing of a vast number of possible candidate molecules, combined with an even larger number of possible permutations of candidate molecular reactions, reaction conditions, ratios, and other considerations. Exhaustive testing of the sheer number of possibilities would be impractical, indeed infeasible, through human effort. By providing training and use of a machine learning model, the present disclosure facilitates the prediction of target molecular products, reactions, reaction conditions, synthons, instances, etc., as well as the adaptive identification of elements having poor performance for optimization. In this way, the processes of compound development, synthesis, optimization, and/or screening are made more rapid and efficient, thus improving the technical field of drug discovery.

In some embodiments, the presently disclosed systems and methods provide for an automated reaction device in combination with a machine learning model that improves the accuracy, reliability, and reproducibility of the molecular outputs (e.g., molecular products, reactions, reaction conditions, synthons, and/or instances thereof), for at least the reasons noted above, thereby improving the technical field of drug discovery.

Accordingly, the present disclosure provides systems and methods for improving molecular reaction conversion values for a set of synthons. An initial conversion value for the synthons is obtained for an initial reaction instance that transforms the synthons into compounds under initial reaction conditions using an automated device. When the initial conversion value fails to satisfy a selection criterion, the synthons are optimized by performing test reaction instances using the synthons, where each test instance includes a corresponding set of normalized conditions. A test conversion value is determined for each test instance. Each test instance having a test conversion value that satisfies the criterion is selected. In some embodiments, a selected test instance is further used for optimization of one or more reaction conditions, in a corresponding set of reaction conditions for the selected test instance. Another aspect of the present disclosure provides systems and methods for selecting synthon sets for optimization of a molecular reaction. Another aspect of the present disclosure provides systems and methods for determining synthons having target conversion values when transformed by a molecular reaction.

Still another aspect of the present disclosure provides systems and methods for improving conversion values using multistep molecular reactions. Yet another aspect of the present disclosure provides systems and methods for selecting reaction conditions for use in a multistep molecular reaction.

### Definitions

It will be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “conversion value” is determined as a percent consumption (e.g., mass, volume) of one or more synthons and/or one or more reactants (e.g., a first reactant and/or a second reactant) obtained in a molecular reaction. As an example, where 80% of a first synthon and/or a first reactant is consumed in the respective instance of the molecular reaction, the percent consumption of the first synthon and/or the first reactant is 80%. In some embodiments, a conversion value is calculated using actual values of the corresponding compound (reaction product) and/or one or more synthons and/or one or more reactants (e.g. by directly measuring the remaining amounts after synthesis of the target compound). In some embodiments, a conversion value is calculated using estimated values of the target compound and/or one or more synthons and/or one or more reactants (e.g. by using methods capable of determining concentrations such as UV spectroscopy).

In some embodiments a conversion value is a metric for reaction efficiency, such as percent yield, conversion, selectivity, or atom economy. Yield is the amount of product formed relative to the theoretical maximum amount that could be formed, often expressed as a percentage. There are two main types of yield, theoretical yield and actual yield. Theoretical yield is the maximum amount of product expected based on reaction stoichiometry. Actual yield is the amount of product actually obtained from the reaction. In some embodiments percent yield is defined as:

$$( \frac{actual yield}{theoretical yield} ) \times 100 \% .$$

Conversion is the fraction of reactant that has been converted into product. It is often used in industrial and catalytic processes. In some embodiments conversion is defined as:

$$( \frac{Initial amount of reactant - Remaining amount of reactant}{Initial amount of reactant} ) \times 100 \% .$$

Selectivity is the ratio of the desired product formed to the total products formed. In some embodiments selectivity is defined as:

$$( \frac{Desired product}{Total products} ) \times 100 \% .$$

Atom economy is a measure of the efficiency of a reaction in terms of how well atoms are utilized. In some embodiments atom economy is defined as:

$$( \frac{Molecular weight of desired product}{Molecular weight of all reactants} ) \times 100 \% .$$

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “hour” is an epoch of 3,600 seconds. Moreover, as used herein, the term “day,” is an epoch of 86,400 seconds.

As used interchangeably herein, the terms “macromolecule,” “macromolecule complex,” or “polymer” refer to a biological object that is capable of interacting with a molecule. In some embodiments, a macromolecule is a protein, a polypeptide, a polynucleic acid, a polyribonucleic acid, a polysaccharide, or an assembly of any combination thereof. In some embodiments, a macromolecule is a large molecule composed of repeating residues. In some embodiments, the macromolecule is a natural material. In some embodiments, the macromolecule is a synthetic material. In some embodiments, the macromolecule is an elastomer, shellac, amber, natural or synthetic rubber, cellulose, Bakelite, nylon, polystyrene, polyethylene, polypropylene, polyacrylonitrile, polyethylene glycol, or a polysaccharide. In some embodiments, the macromolecule is a heteropolymer (copolymer). In some embodiments, the macromolecule is a plurality of polymers (e.g., 2 or more, 3, or more, 10 or more, 100 or more, 1000 or more, or 5000 or more polymers), where the respective polymers in the plurality of polymers do not all have the same molecular weight. In some embodiments, the macromolecule is a polypeptide. As used herein, the term “polypeptide” means two or more amino acids or residues linked by a peptide bond.

In some embodiments, the macromolecule includes any number of posttranslational modifications. Thus, in some embodiments, a macromolecule includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphatases and kinases). Other types of posttranslational modifications are known in the art and are within the scope of the macromolecules or macromolecule complexes of the present disclosure.

In some embodiments, the macromolecule is a surfactant. In some embodiments, the macromolecule is a reverse micelle or liposome. In some embodiments, the target macromolecule is a fullerene. In some embodiments, the macromolecule includes two different types of polymers, such as a nucleic acid bound to a polypeptide. In some embodiments, the target macromolecule includes two polypeptides bound to each other. In some embodiments, the target macromolecule includes one or more metal ions (e.g., a metalloproteinase with one or more zinc atoms).

As used herein, the term “normalized condition” refers to a condition associated with a reaction. Nonlimiting examples of normalized conditions include synthon type, a reagent, a solvent, a concentration thereof, an order of reactant addition, an amount of equivalents for addition, synthon scope, temperature, incubation time, stoichiometry of synthons, and stoichiometry of reagents. Alternatively or additionally, in some embodiments, a normalized condition is an experimental layout (e.g., on a reaction plate) that hosts a reaction. In some embodiments, the molecular reaction, and/or one or more instances thereof, is performed in a reaction plate, including, but not limited to, a 12-well, 24-well, 48-well, 96-well, and/or 384-well plate. In some embodiments, a normalized condition is one or more solvents suitable for use in automation. In some embodiments, a solvent having a boiling point, rate of evaporation, density, and/or surface tension the same or substantially the same or greater than that of water would be suitable for use in automation, whereas a solvent having a boiling point, a rate of evaporation, density, and/or surface tension less than that of water would not be ideal for use in automation.

As used herein, the term “model” refers to a machine learning model, algorithm, model, regressor, and/or classifier.

In some embodiments, a model is an unsupervised model. One example of an unsupervised model is cluster analysis.

In some embodiments, a model is a supervised model. Nonlimiting examples of supervised learning models include, but are not limited to, logistic regression, neural networks, support vector machines, Naive Bayes algorithms, nearest neighbors, random forests, decision trees, boosted trees, multinomial logistic regression, linear models, linear regression, GradientBoosting, mixture models, hidden Markov models, Gaussian NB, linear discriminant analysis, or any combinations thereof. In some embodiments, a model is a multinomial classifier or regressor. In some embodiments, a model is a 2-stage stochastic gradient descent (SGD) model. In some embodiments, a model is a deep neural network (e.g., a deep-and-wide sample-level classifier).

Neural networks. In some embodiments, the model is a neural network (e.g., a convolutional neural network and/or a residual neural network). Neural networks, also known as artificial neural networks (ANNs), include convolutional and/or residual neural networks (deep learning models). Neural networks can be machine learning models that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. In some embodiments, a deep learning model is a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. In one example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in the present disclosure. Examples can include, but are not limited to, feedforward neural networks, radial basis function networks, recurrent neural networks, residual neural networks, convolutional neural networks, residual convolutional neural networks, and the like, or any combination thereof.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural networks, including convolutional neural networks, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVMs suitable for use as models of the present disclosure are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes model. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor model. Nearest neighbor models can be memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses any type of regression. For example, in some embodiments, the regression is logistic regression. In some embodiments, the regression is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve model performance. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, the model is a reinforcement learning model. In some embodiments, the reinforcement learning model comprises four main elements—an agent, a policy, a reward signal, and a value function, where the behavior of the agent is defined in terms of the policy. In some embodiments, the reinforcement learning model comprises a learning algorithm. In some implementations, the learning algorithm is an on-policy learning algorithm or an off-policy learning algorithms. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety. In some embodiments, the reinforcement learning model includes at least 10, at least 100, at least 1000, at least 10,000, at least 100,000, at least 1×10⁶, at least 1×10⁷, or more parameters. In some embodiments, the reinforcement learning model includes no more than 1×10⁸, no more than 1×10⁷, no more than 1×10⁶, no more than 100,000, no more than 10,000, no more than 1000, or no more than 100 parameters. In some embodiments, the reinforcement learning model consists of from 10 to 1000, from 100 to 100,000, from 10,000 to 1×10⁷, or from 1×10⁶ to 1×10⁸ parameters. In some embodiments, the plurality of parameters for the reinforcement learning model falls within another range starting no lower than 10 parameters and ending no higher than 1×10⁸ parameters.

As used herein, the term “parameter” refers to any coefficient or, similarly, any value of an internal or external element (e.g., a weight and/or a hyperparameter) in an algorithm, model, regressor, and/or classifier that affects (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that is used to control, modify, tailor, and/or adjust the behavior, learning and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable an algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods, as described elsewhere herein).

In some embodiments, an algorithm, model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal Instruction Set Computers (MISC), Very Long Instruction Word (VLIW), Explicitly Parallel Instruction Computing (EPIC), and One Instruction Set Computer (OISC).

As used herein, “synthon” refers to a representation of a chemical structure having an open valence (attachment bond) at least at one position. In embodiments, synthons are derived from a reagent, from a synthetic reaction sequence, or from the fragmentation of a molecule (e.g., chemical structures derived from the disconnection of a bond). In embodiments, synthons are used to computationally assemble a whole molecule, or when appropriate through synthetic organic chemistry, to synthesize a whole molecule.

### Example Systems for Improving Models for Use in Optimizing Molecular Reactions

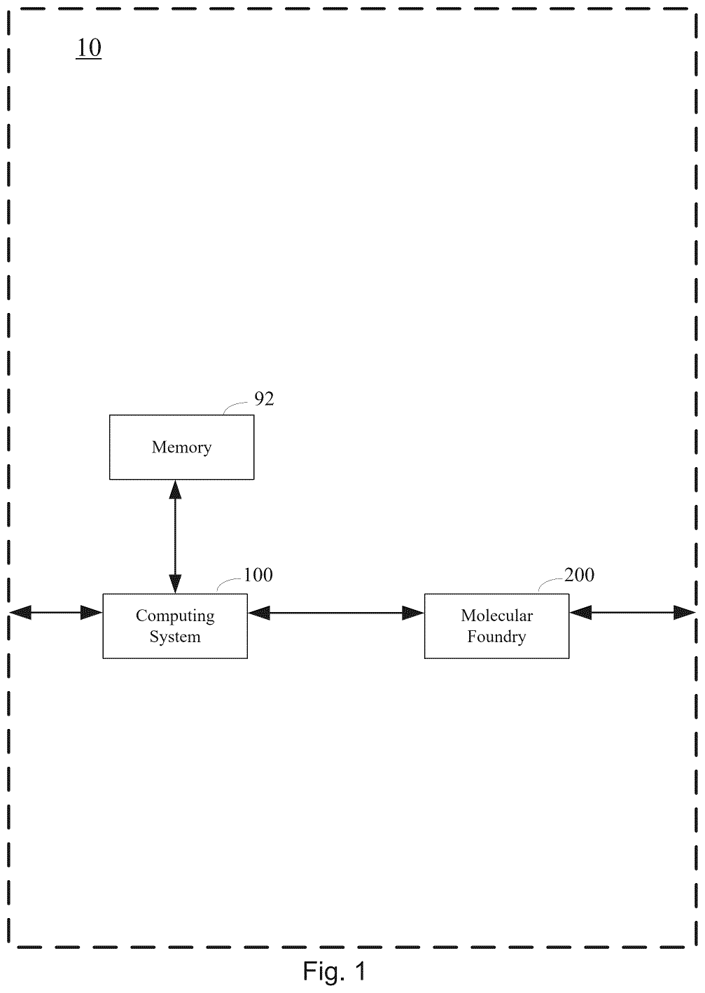

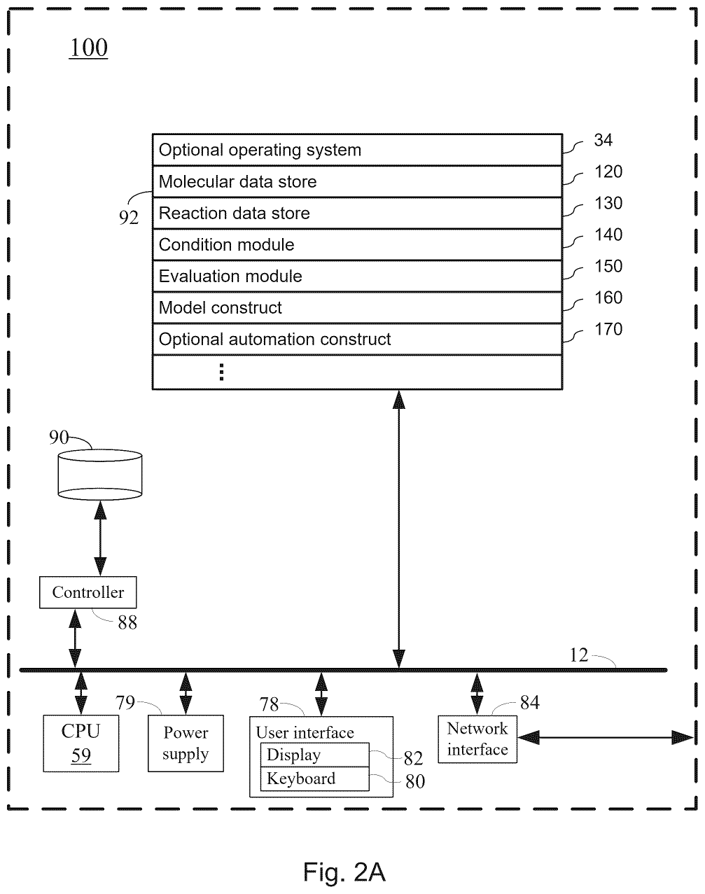

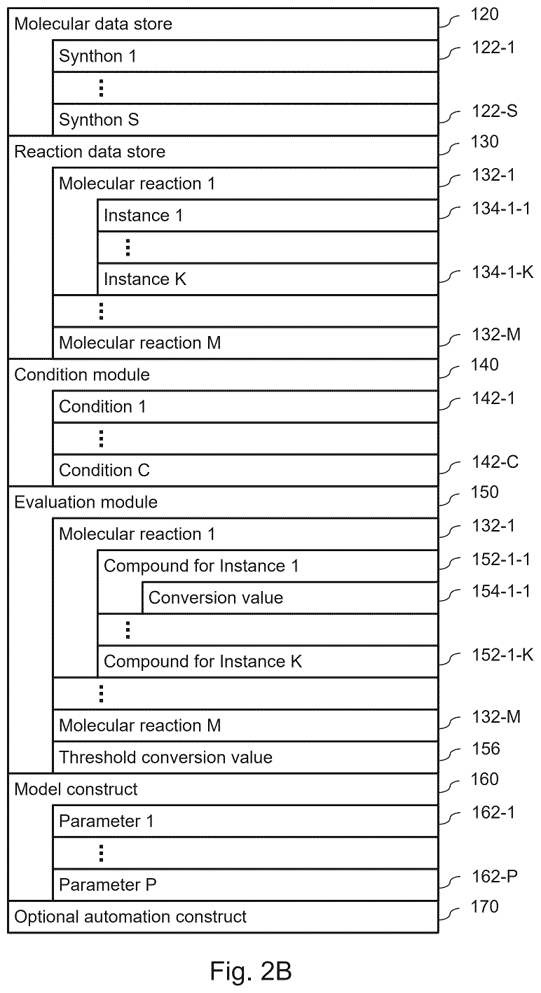

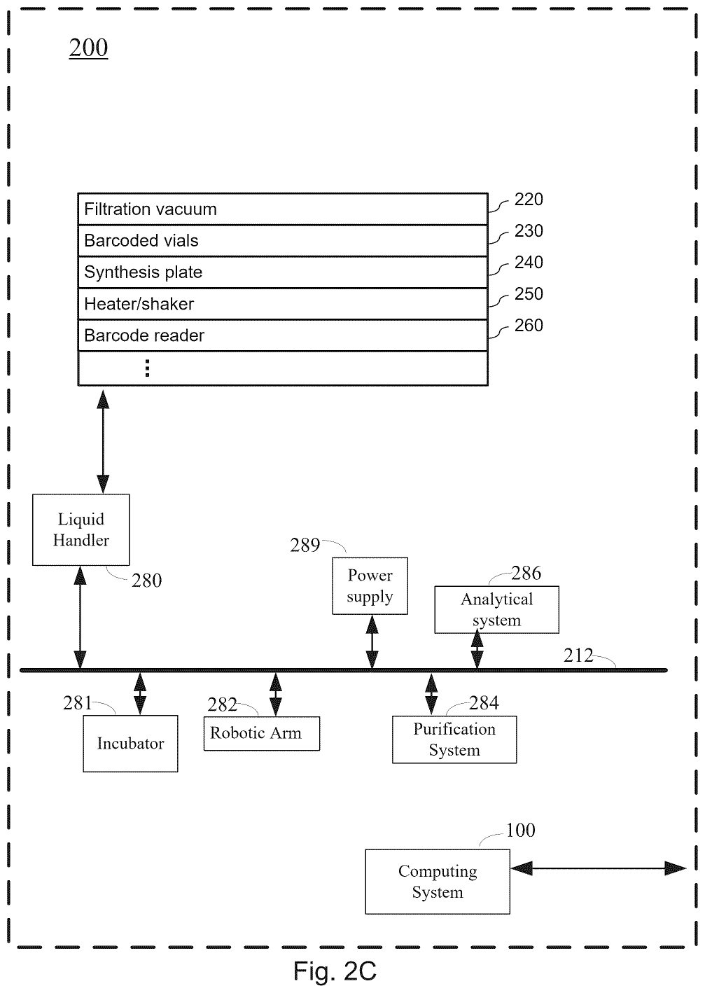

FIGS. 1-2B collectively illustrate a computer system 100 (e.g., for improving a conversion value of a molecular reaction, selecting a set of synthons for optimization of a molecular reaction, and/or selecting reaction conditions for use in a molecular reaction, such as a multistep molecular reaction).

FIGS. 2A-2B, in some embodiments, computing system 100 comprises one or more computers. For purposes of illustration in FIGS. 2A-2B, the computing system 100 is represented as a single computer that includes all of the functionality of the disclosed computing system 100. However, the present disclosure is not so limited. The functionality of the computing system 100 can be spread across any number of networked computers and/or reside on each of several networked computers and/or virtual machines. One of skill in the art will appreciate that a wide array of different computer topologies is possible for the computer system 100 and all such topologies are within the scope of the present disclosure.

The computer system 100 comprises one or more processing units (CPUs) 59, a network or other communications interface 84, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

In some embodiments, the memory 92 of the computing system 100 stores:
an optional operating system 34 that includes procedures for handling various basic system services;
a molecular data store 120, optionally comprising a plurality of synthons 122 (e.g., 122-1, . . . 122-S);
a reaction data store 130, optionally comprising one or more molecular reactions 132 (e.g., 132-1, . . . 132-M) and, for a respective selected molecular reaction 132, a plurality of instances 134 (e.g., 134-1-1, . . . 134-1-K);
a condition module 140, optionally comprising a plurality of conditions 142 (e.g., 142-1, . . . 142-C), where:
   the plurality of instances 134 of the molecular reaction 132 (e.g. a first molecular reaction or a second molecular reaction) are performed using the plurality of synthons 122 (e.g., at least 4 synthons) and the plurality of conditions 142 (e.g. an original plurality of normalized conditions), thereby generating a plurality of compounds 152 (e.g., 152-1-1, . . . 152-1-K), and
   for each respective instance 134 of the molecular reaction 132, at least a subset of the plurality of synthons 122 are transformed;
an evaluation module 150, optionally comprising, for each respective instance 134 of the molecular reaction 132, a respective conversion value 154 for the respective instance (e.g., 154-1-1) and a threshold conversion value 156, where:
   a subset of instances 134 are selected from the plurality of instances based on at least the threshold conversion value 156 and the respective conversion value 154 for each respective instance;
a model construct 160, optionally comprising a plurality of parameters 162 (e.g., 162-1, . . . 162-P), where:
   the subset of instances 134 are used to adjust one or more parameters in the plurality of parameters 162 of the model construct 160, thereby obtaining an updated plurality of parameters, and
   the model construct 160 uses, subsequent to obtaining the updated plurality of parameters 162, to search for and identify an updated plurality of normalized conditions 164 for the molecular reaction 132 that collectively have an improved conversion value for the molecular reaction 132 relative to the plurality of conditions 142;
a training construct 170, where:
   the subset of instances 134 are used as a first set of variables (e.g., independent variables) and the respective conversion value 154 for each respective instance are used as a second set of variables (e.g., dependent variables), and
   the training construct 170 uses the subset of instances 134 and the respective conversion value 154 for each respective instance to guide adjustment of one or more parameters in a plurality of parameters 162 of the model construct 160, so that the model construct 160 produces a calculated conversion value in agreement with the corresponding conversion value of each instance of the subset of instances 134 upon input of the subset of instances 134 into the model construct 160; and
a selection construct 180, optionally comprising a first subset of synthons 158, where:
   the subset of instances 134 are used as independent variables and the respective conversion value 154 for each respective instance are used as dependent variables, and selecting a first subset of synthons that are enriched in the first subset of instances 158 relative to the plurality of instances of the molecular reaction.

In some implementations, one or more of the above identified data elements or modules of the computer system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above. In some embodiments, the first neural network 72 is replaced with another form of model.

Referring to FIG. 2C, in some embodiments, a molecular foundry 200 comprises one or more automated synthesis platforms. In some embodiments, the molecular foundry 200 is included in an apparatus of the disclosure. For purposes of illustration in FIGS. 1-2C, the molecular foundry 200 is represented as a single automated synthesis platform that includes all of the functionality of the disclosed molecular foundry 200. However, the present disclosure is not so limited. The functionality of the molecular foundry 200 can be spread across any number of networked automated synthesis platform. One of skill in the art will appreciate that a wide array of different automated synthesis platform topologies is possible for the molecular foundry 200 and all such topologies are within the scope of the present disclosure.

The molecular foundry 200 comprises one or more computing systems 100, a liquid handler 280, an incubator 281, a robotic arm 282, a purification system 284, an analytical system 286; one or more communication busses 212 for interconnecting the aforementioned components, and a power supply 289 for powering the aforementioned components.

In some embodiments, the liquid handler 280 comprises:
a filtration vacuum 220;
one or more barcoded vials 230;
a synthesis plate 240;
a heater/shaker 250; and
a barcode reader 260.

In some embodiments, the molecular foundry is useful for improving a model 160 for use in an optimization of a molecular reaction. In some embodiments, the apparatus comprises an automated synthesis platform (e.g., automated synthesis platform) and a computing system (e.g., computing system 100). In some embodiments, the computing system comprises one or more processors and memory addressable by the one or more processors, the memory storing the model. In some embodiments, the computing system 100 informs the molecular foundry 200 of the molecular reaction (see, for example, FIG. 1).

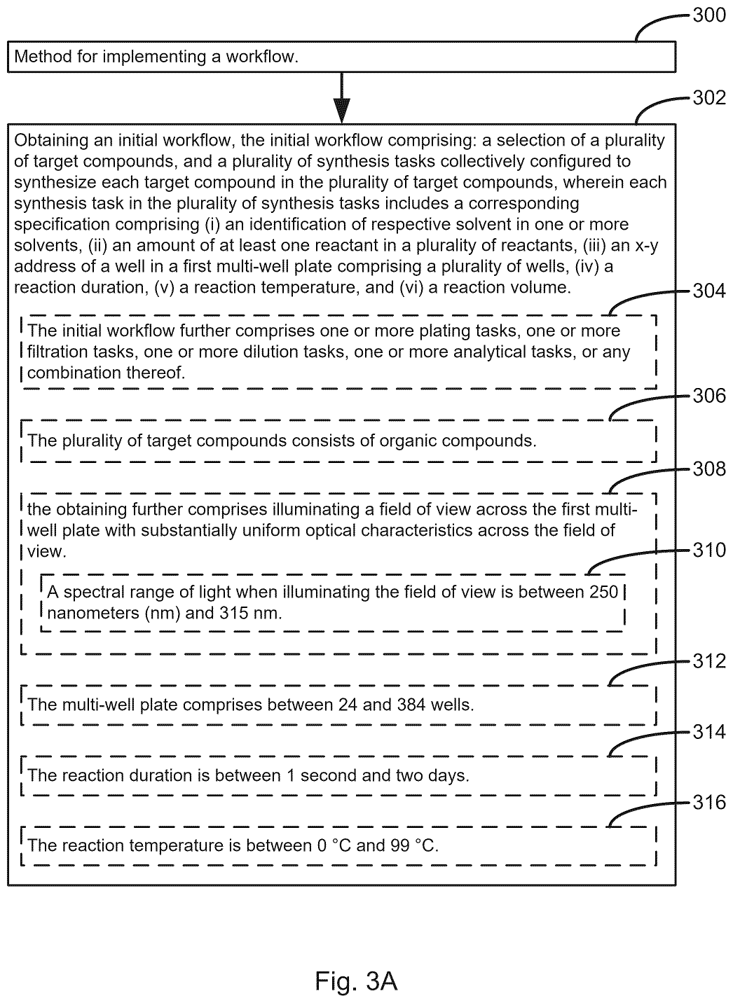

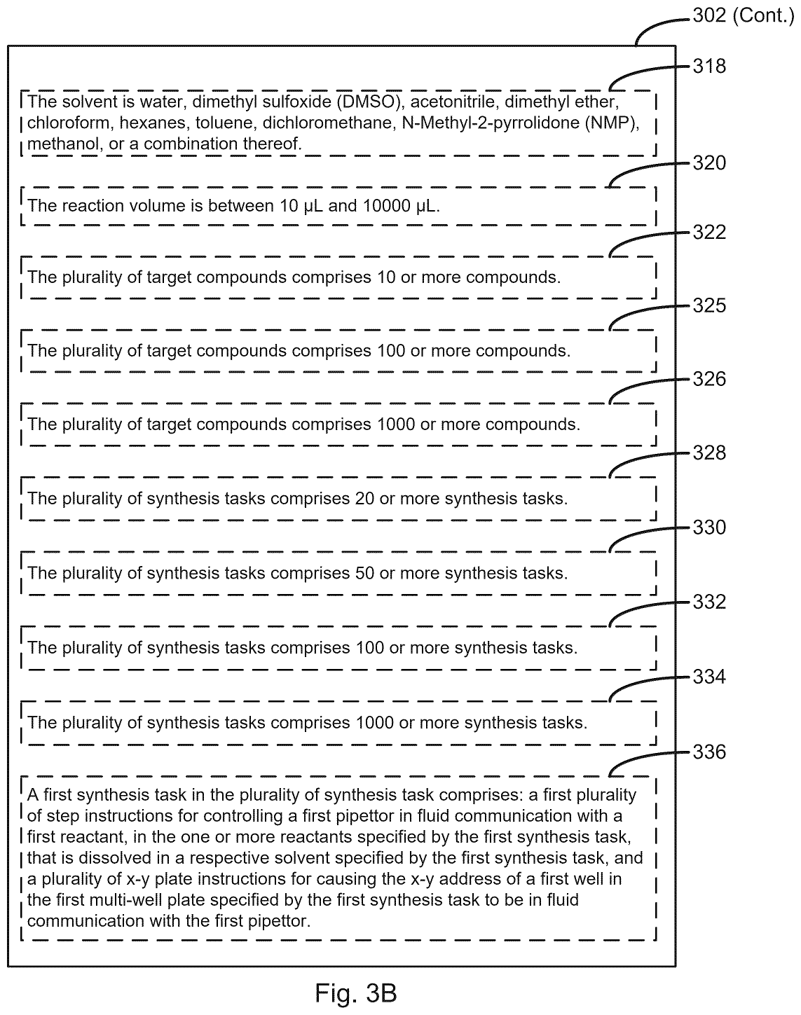

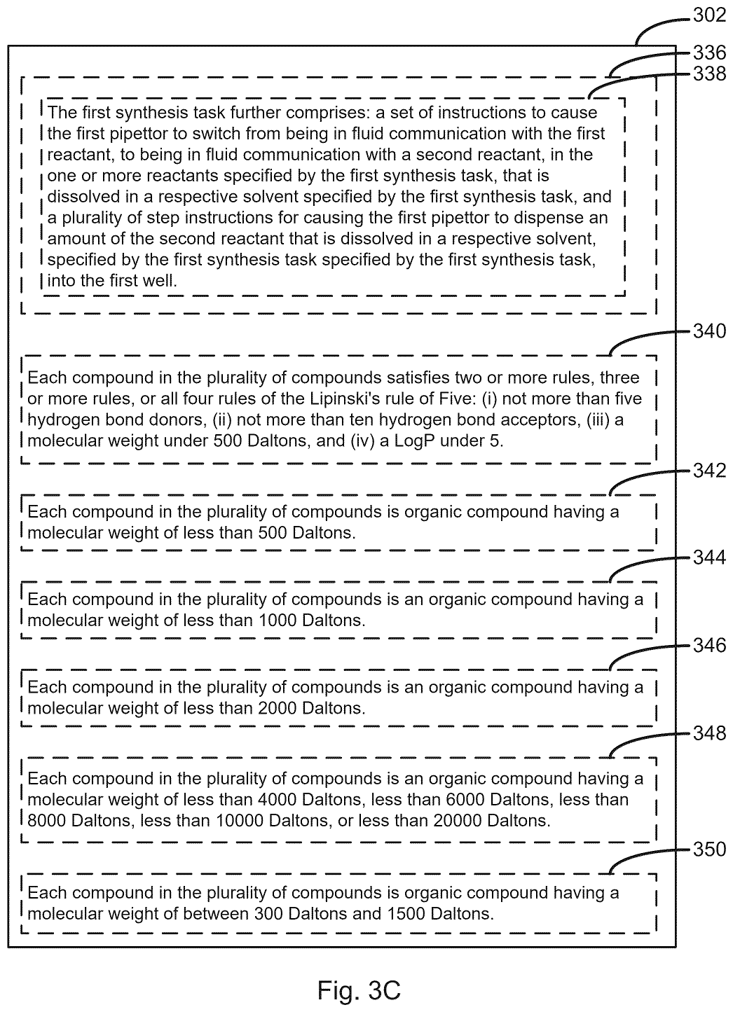

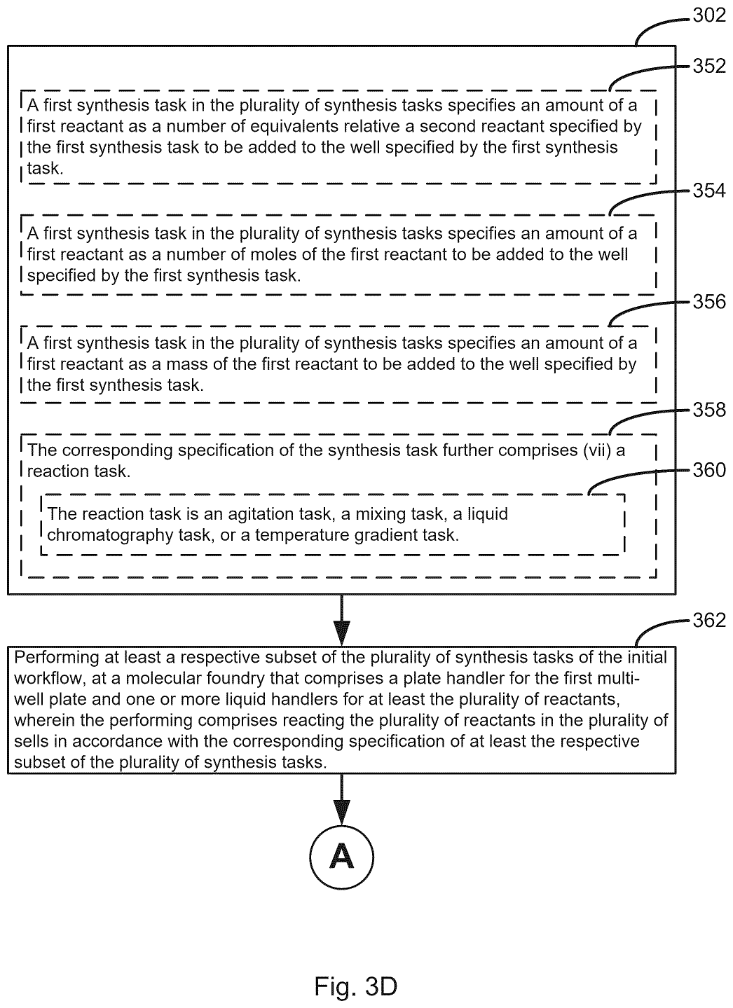

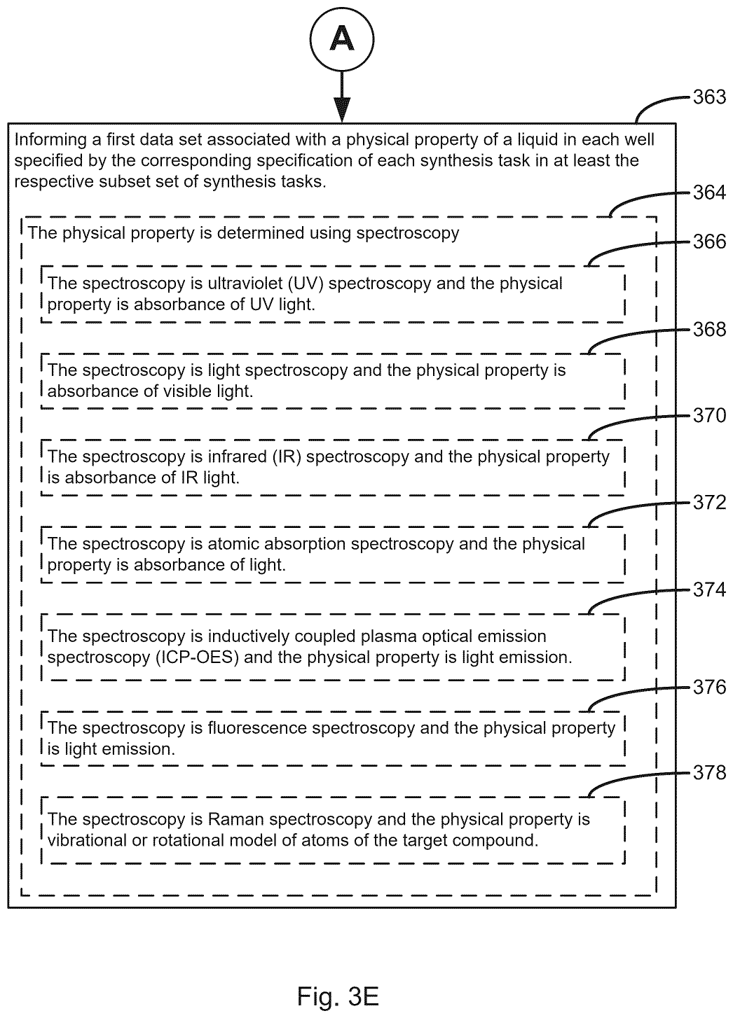

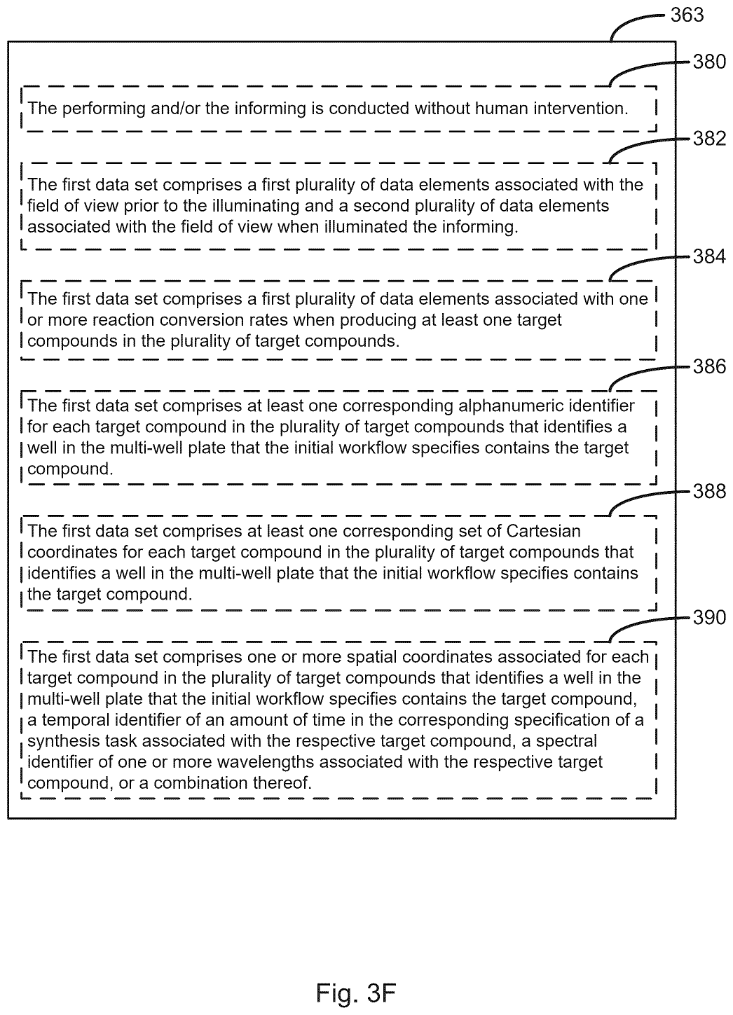

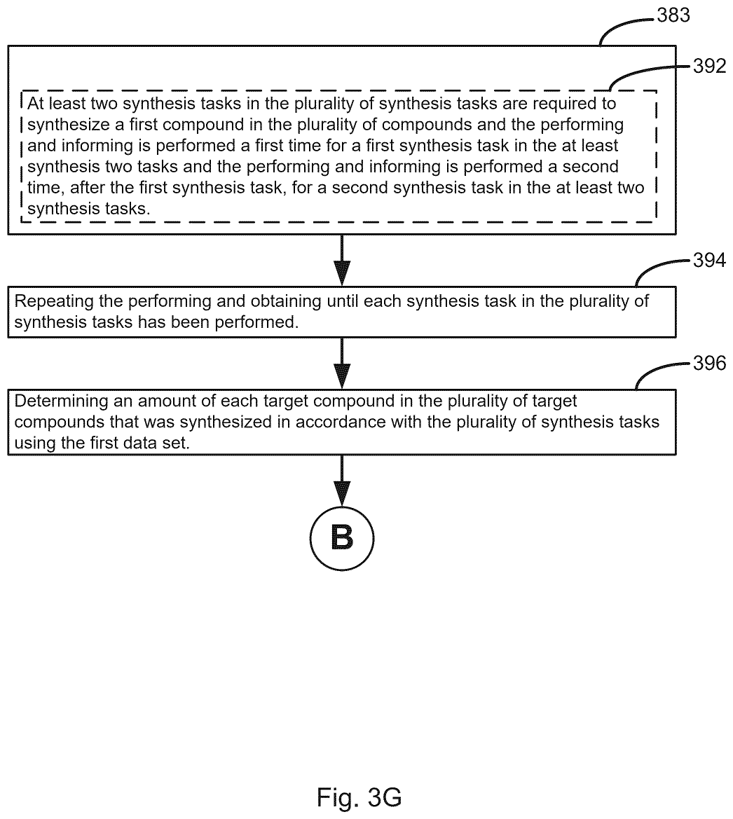

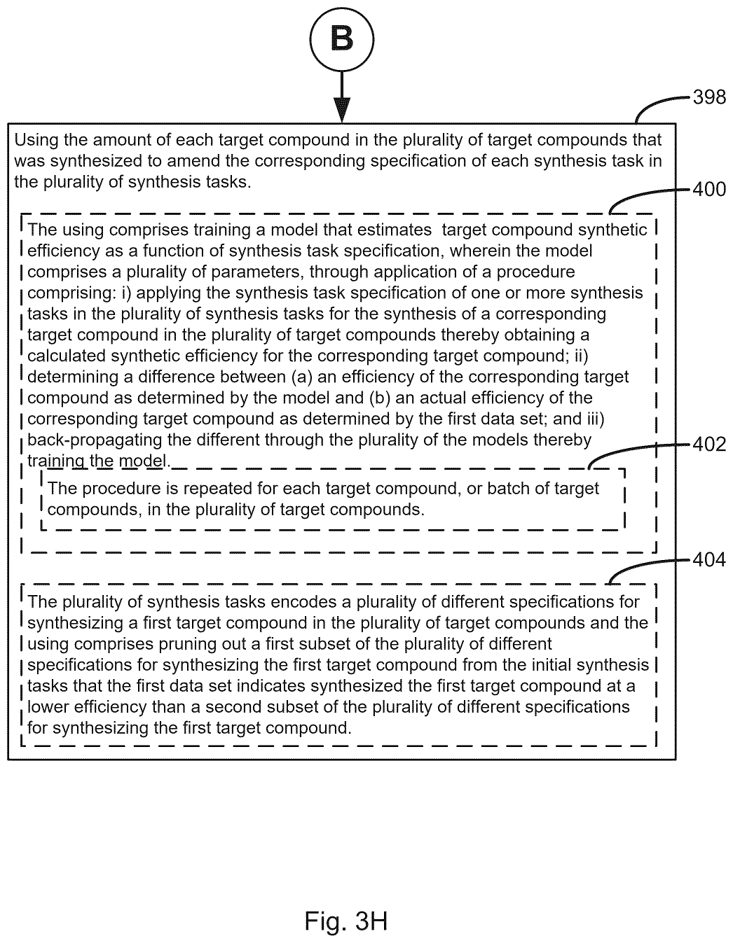

Now that a system 100 (e.g., for improving a conversion value of a molecular reaction, selecting a set of synthons for optimization of a molecular reaction, and/or selecting reaction conditions for use in a molecular reaction, such as a multistep molecular reaction) has been disclosed, methods for performing such processes are detailed with reference to FIGS. 3A-7E.

### Example Methods for Implementing a Workflow

As illustrated in FIGS. 3A-3H, another aspect of the present disclosure provides a method 300 for automating synthesis of a compound using a molecular reaction. In some embodiments, the molecular reaction is a multistep molecular reaction.

In some embodiments, the molecular reaction comprises at least 2, at least 3, or at least 4 steps. In some embodiments, the molecular reaction comprises at least 5, at least 10, at least 20, or at least 30 steps. In some embodiments, the molecular reaction comprises no more than 50, no more than 30, no more than 20, no more than 10, or no more than 5 steps. In some embodiments, the molecular reaction consists of from 2 to 5, from 2 to 10, from 5 to 20, from 10 to 30, or from 20 to 50 steps. In some embodiments, the molecular reaction falls within another range starting no lower than 2 steps and ending no higher than 50 steps.

In some embodiments, the molecular reaction is a first molecular reaction (e.g., a molecular reaction type) selected from a plurality of molecular reactions (e.g., a plurality of molecular reaction types). In some embodiments, the molecular reaction is a second molecular reaction (e.g., a molecular reaction type) selected from a plurality of molecular reactions (e.g., a plurality of molecular reaction types).

In some embodiments, the plurality of molecular reactions comprises at least 2, at least 5, at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 2 molecular reactions and ending no higher than 5000 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, the plurality of molecular reactions comprises one or more reaction SMARTS (SMILES arbitrary target specification). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups (see, Green and Wuts, Protective Groups in Organic Synthesis, second edition, John Wiley & Sons, Inc., New York, 1991, which is hereby incorporated by reference), total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, esterification reactions (e.g., methyl esterification), hydrolysis of esters, amide synthesis, transamidation, oxidative amidation, nucleophilic aromatic substitution reactions, protecting group addition/removal reactions (e.g., additional/removal of tert-butoxycarbonyl protecting group (BOC group)); addition/removal of silyl protective group (e.g., trimethylsilyl group, triethylsilyl group, tert-butyldimethylsilyl (TBDMS), tert-butyldiphenylsilyl group (TBDPS)), reaction of electrophiles with amines, synthesis of heterocycles, reductive amination, debenzylation, alkylation of an alcohol (e.g., phenol), sulfonamide formation, reduction (e.g., reduction of nitro group to amine group, reduction of aldehyde, ketone, carboxylic acid, etc., to alcohol), oxidation (e.g., oxidation of an alcohol to an aldehyde, ketone, carboxylic acid, etc.), diazotization followed by reaction with nucleophile, lithiation reaction (e.g., aryl lithiation) followed by reaction with electrophile, halogenation (e.g., aromatic halogenation, aldol reaction, oxidation/reduction of olefin, hydrogenation, oxygenation/deoxygenation, oxidative cleavage reactions, alkylation, hydrolysis and/or decarboxylation of beat-keto ester, Schmidt Reaction, Schotten-Baumann Reaction, Ugi Reaction, arylamine synthesis, Grignard reaction, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki-Miyaura Coupling, Sonogashira Coupling, Click Chemistry, cycloaddition reactions including but not limited to Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, and Synthesis of 1,2,3-Triazoles, Wittig reaction, Horner-Wadsworth-Emmons reaction, epoxide synthesis, Jacobsen-Katsuki Epoxidation, Prilezhaev Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

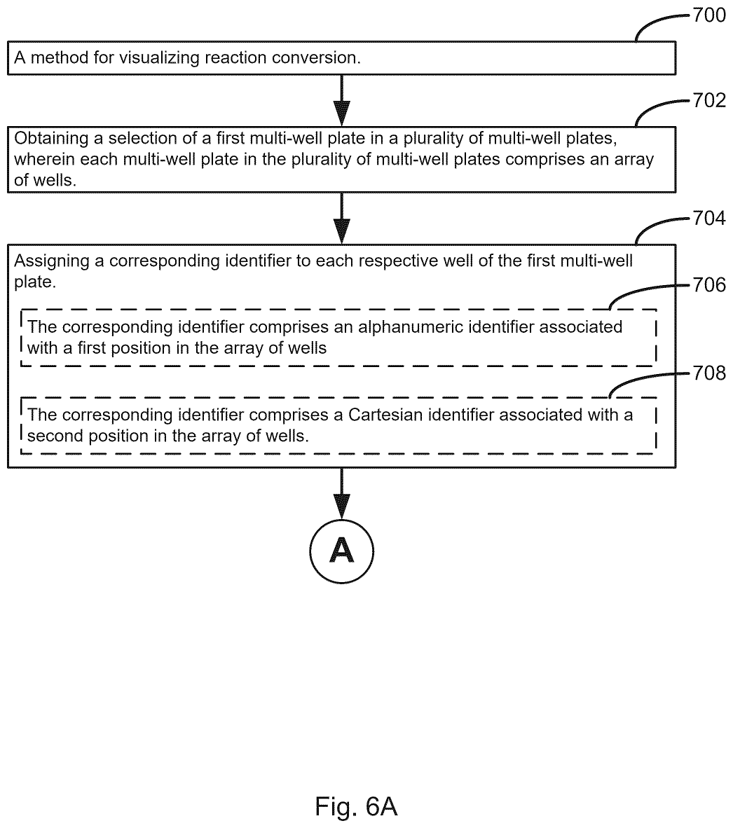

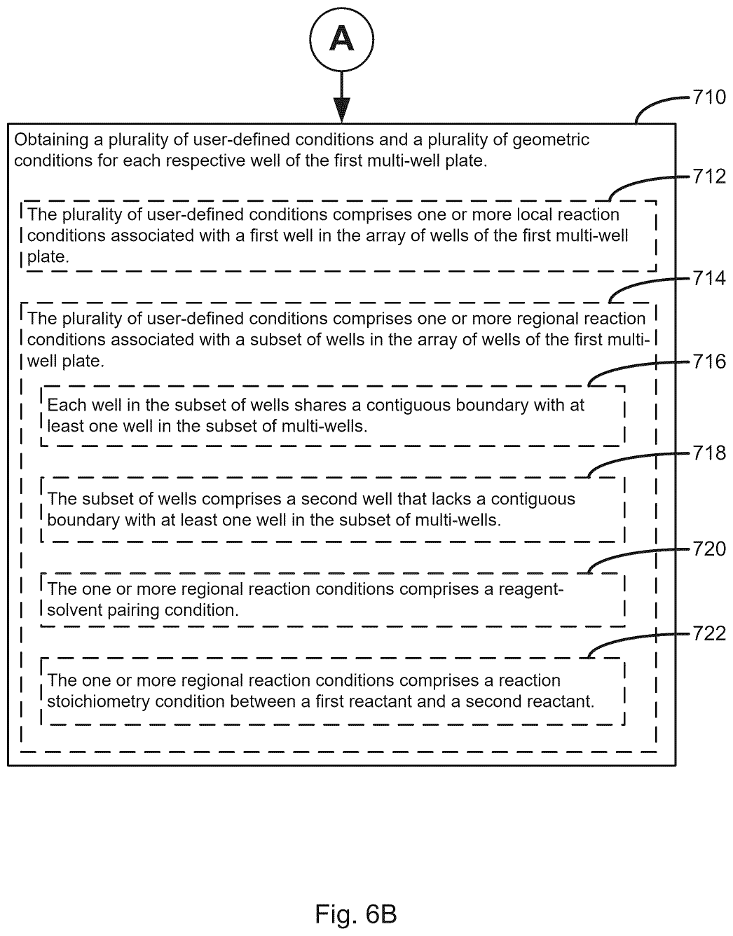

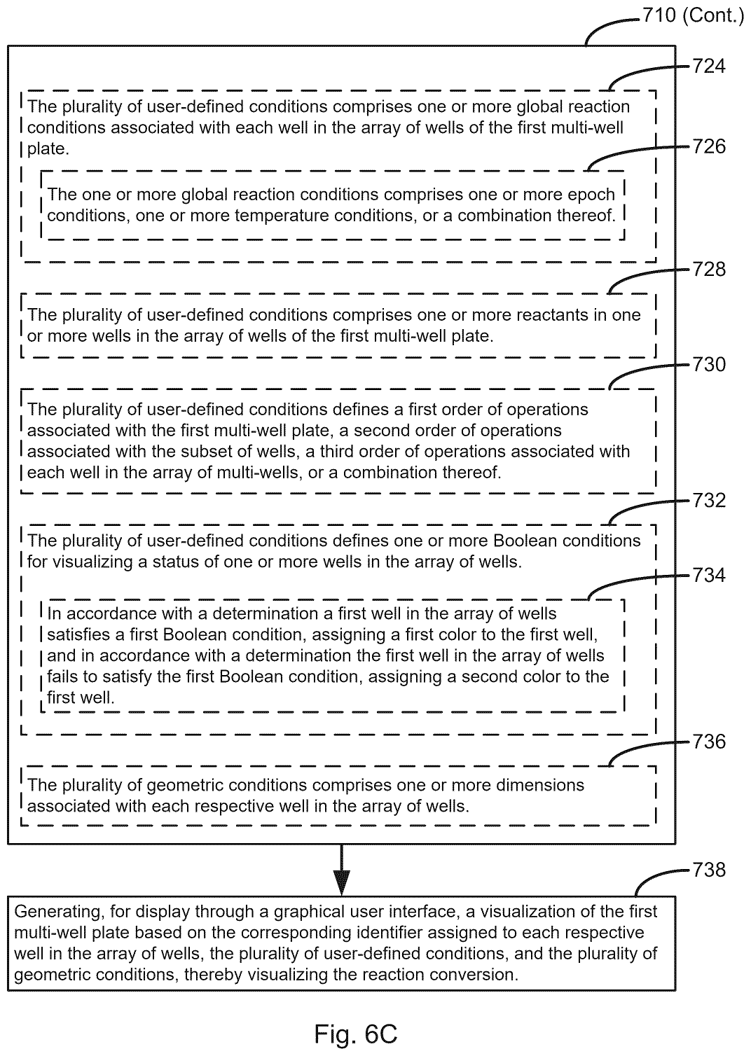

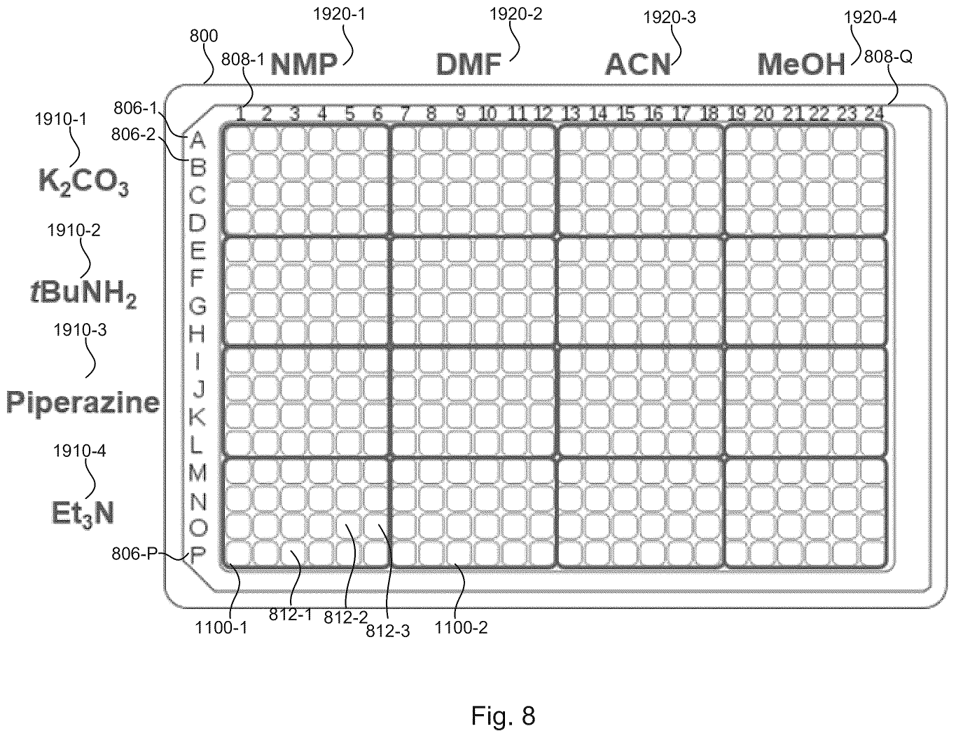

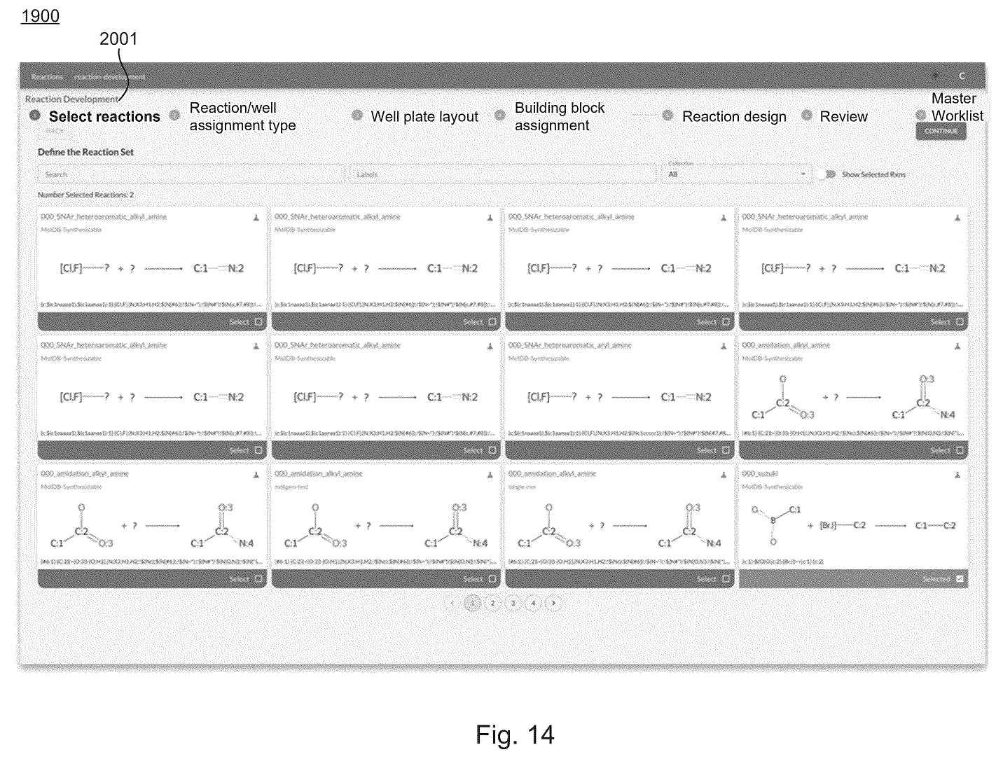

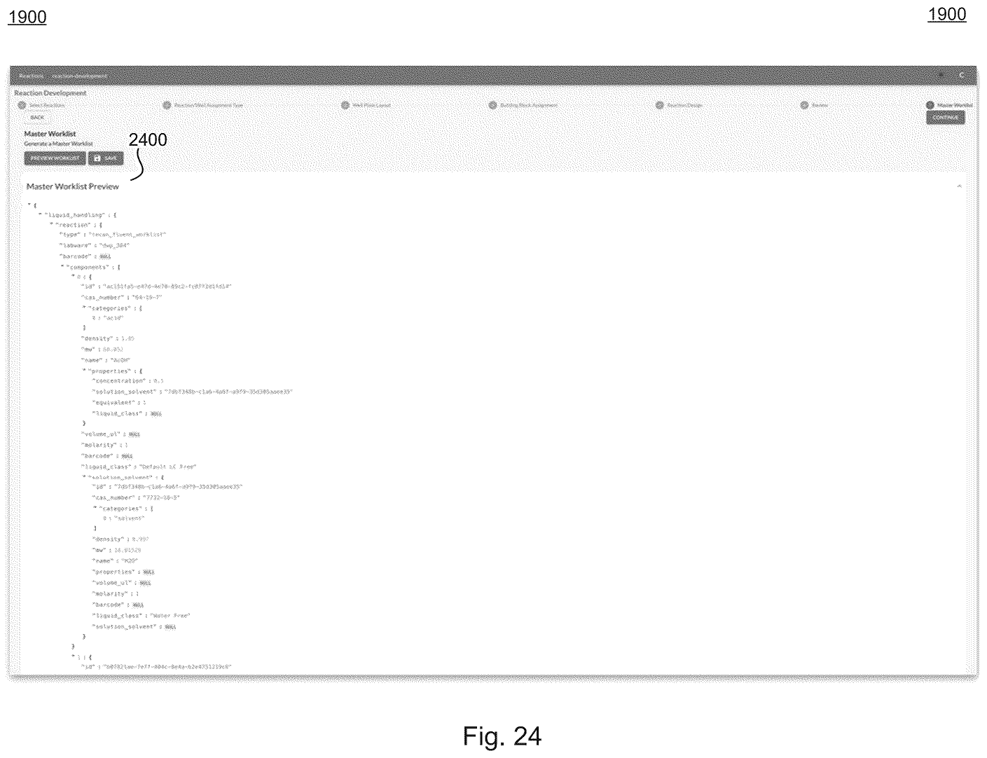

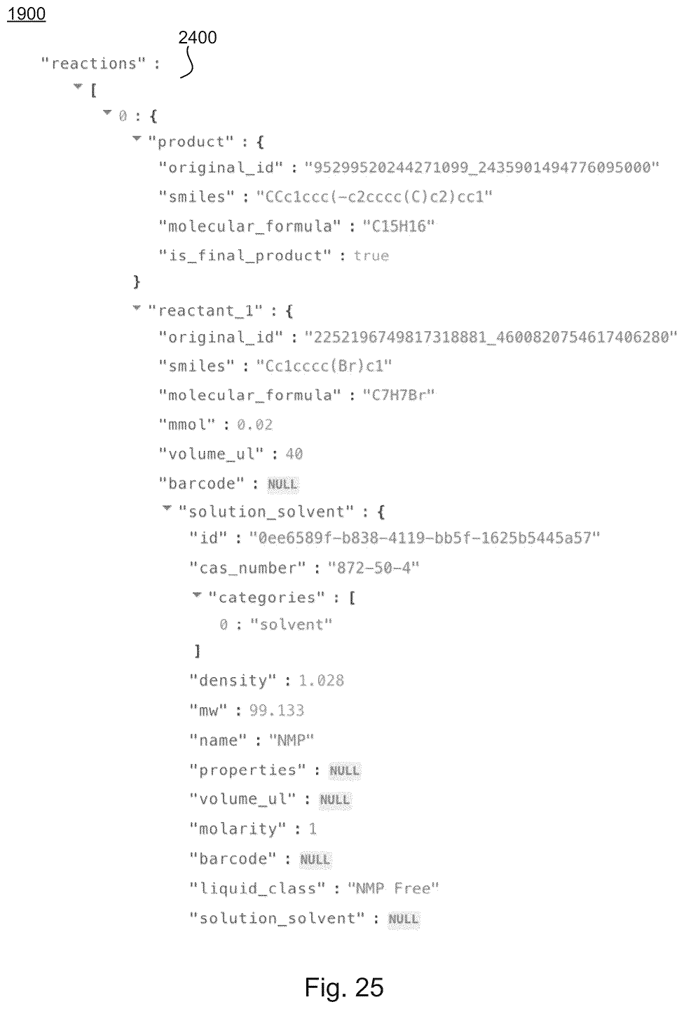

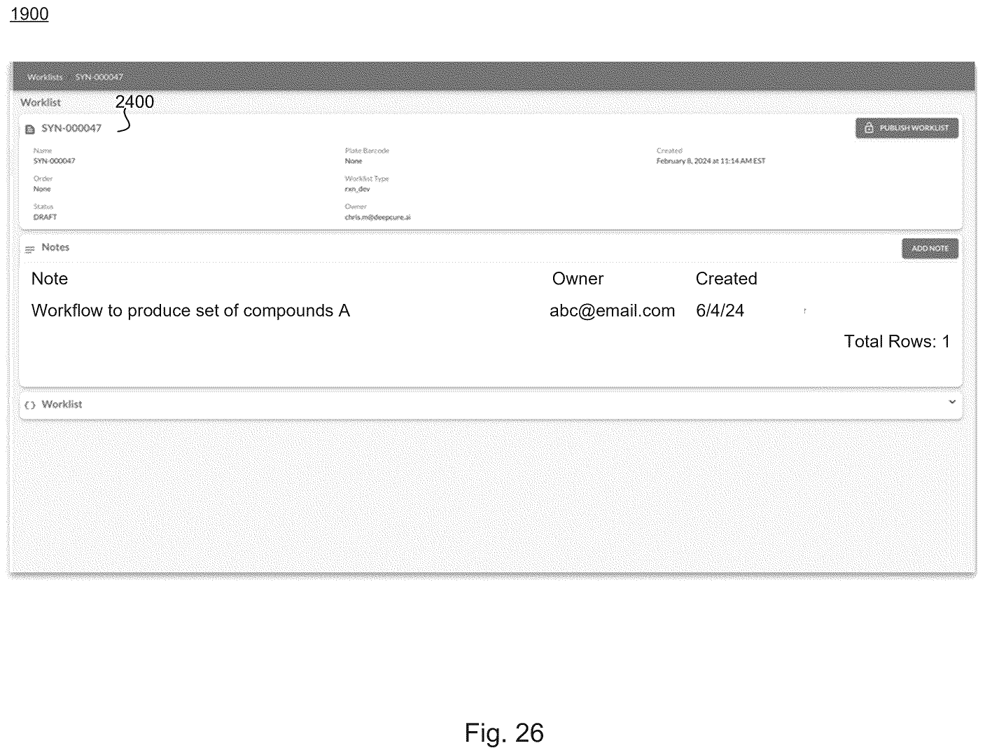

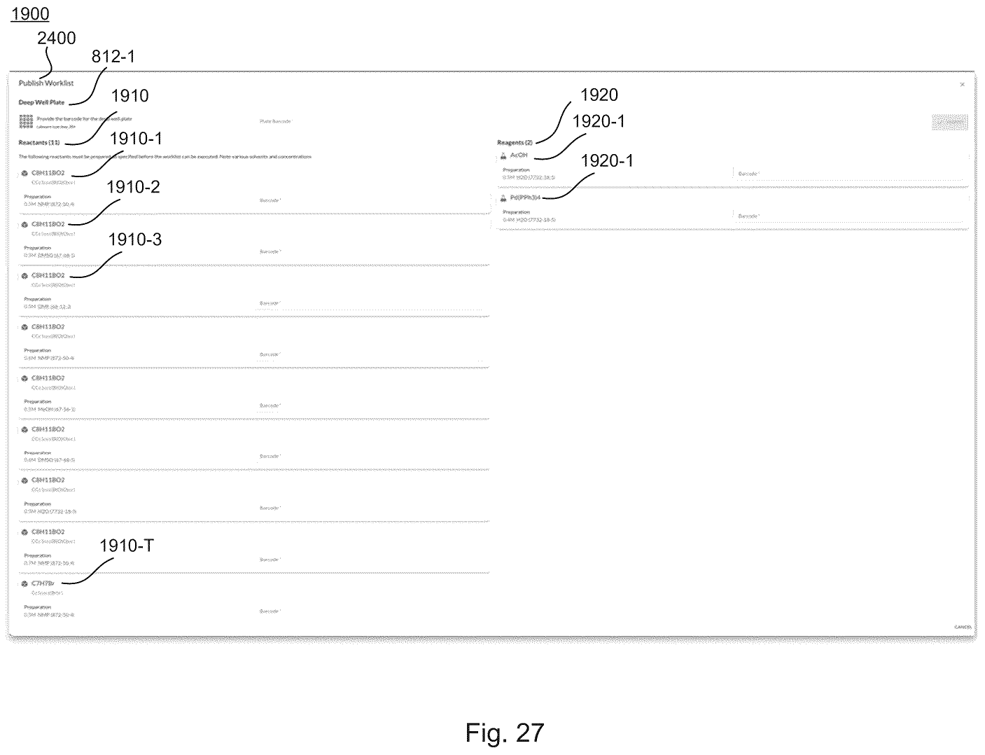

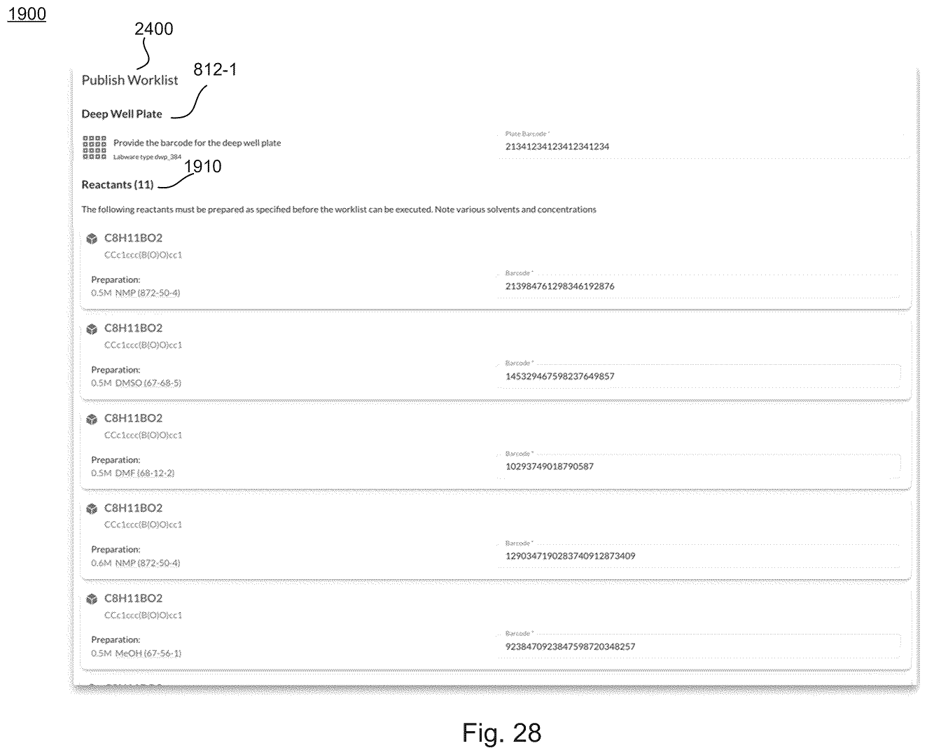

Block 300. Referring to block 300 of FIG. 3A, in some embodiments, a method for implementing a workflow (e.g., workflow of block 602 of FIG. 6A, workflow 2400 of FIG. 24, workflow 2400 of FIG. 25, workflow 2400 of FIG. 26, workflow 2400 of FIG. 27, user interfaces 1900 of FIGS. 8-28, etc.) is provided.

Referring to block 302, in some embodiments, the method 300 includes obtaining an initial workflow 2400, such as a configuration file for a first workflow 2400-1 from a computing system (e.g., computing system 100 of FIGS. 1 and 2A-2B, workflow 2400 of FIGS. 24-28, etc.). For instance, in some embodiments, the initial workflow 2400 is generated at the computing system 100 by designing molecular reaction conditions, instances of molecular reactions (e.g., reaction wells), synthons, and/or molecular products, as well as model inputs or outputs, such as through a user interface of the computing system 100 (e.g., user interface 78 of FIG. 2A, user interfaces 1900 of FIGS. 8-28, etc.). However, the present disclosure is not limit thereto.

In some embodiments, the initial workflow 2400 includes a selection of a plurality of target compounds. For instance, in some embodiments, an end-user selects one or more target compounds for inclusion in the plurality of target compounds through the user interface 78 of the computing system 100. In some embodiments, each well of a multi-well plate 800 is associated with a corresponding target compound in the selection of the plurality of target compounds. However, the present disclosure is not limited thereto.

Moreover, in some embodiments, the initial workflow 2400 includes the selection of a plurality of synthesis tasks that is collectively configured to synthesize each target compound in the plurality of target compounds.

In some embodiments, each workflow 2400 includes one or more respective instances of the molecular reaction that is implementation, replicated, executed, and/or “run” by a computing system 100 associated with a molecular foundry 200. In some embodiments, a first workflow 2400-1 includes a first instance of the molecular reaction that is a replicate of a second instance of the molecular reaction performed by a second workflow 2400-2, where both the first and the second instance of the molecular reaction have the same synthons and/or the same normalized conditions. In some embodiments, a first instance of the molecular reaction and a second instance of the molecular reaction are performed by different workflows having a different set of synthons and/or a different set of normalized conditions. In some embodiments, each respective instance of the molecular reaction has a different set of synthons and/or a different set of normalized conditions from any other instance of the molecular reaction.

In some embodiments, each instance (e.g., each run) of the molecular reaction is performed using a different set of conditions (for instance, to test which conditions result in improved conversion values (e.g., conversion value(s) 154 of FIG. 2, etc.) by permutating the different reaction conditions under which the molecular reaction is performed) defined by the workflow. In some embodiments, the different sets of conditions include one or more different synthons (e.g., selected to be used as starting components for the molecular reaction), and/or one or more different normalized conditions (e.g., reaction conditions such as temperature, incubation time, concentrations, etc., as described above) used to produce a target compound.

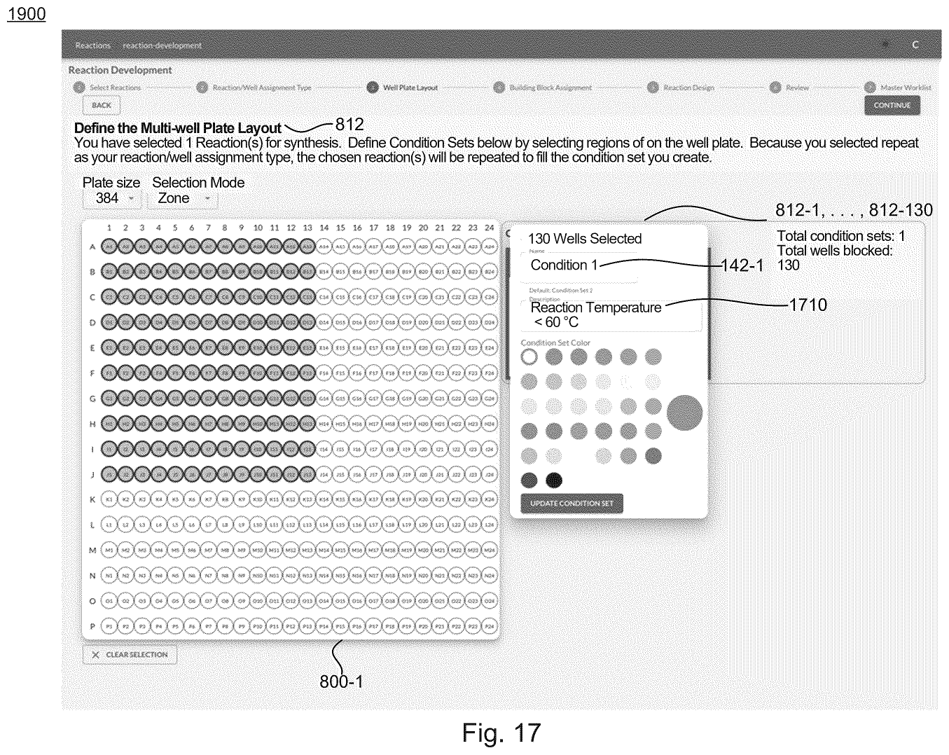

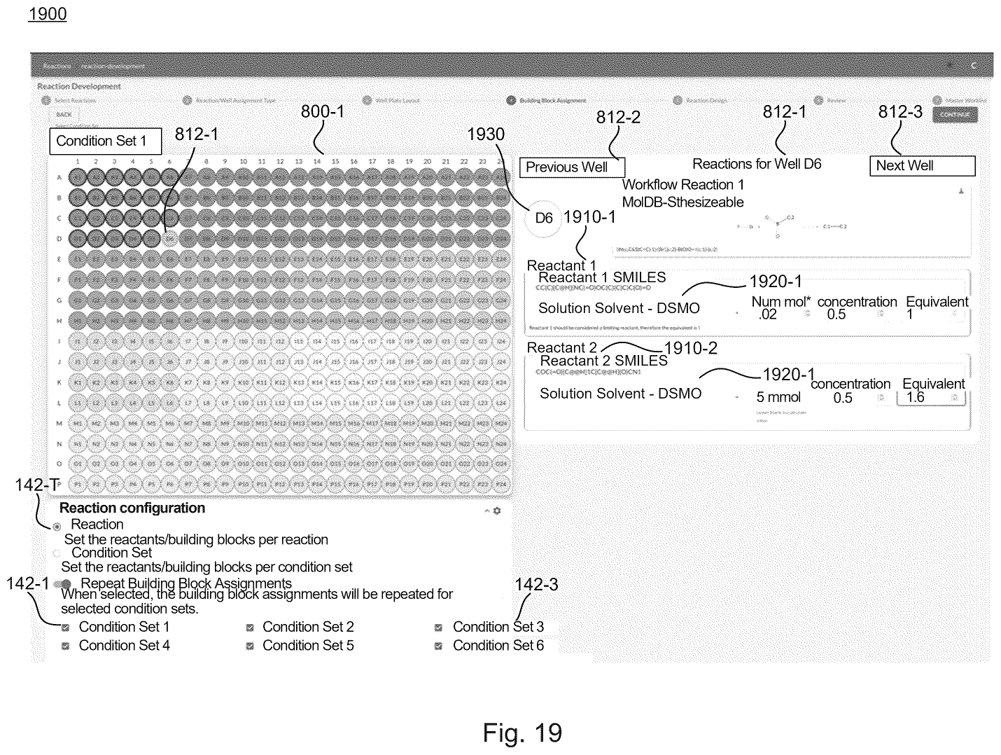

In some embodiments, each synthesis task in the plurality of synthesis tasks includes a corresponding specification, which allows for defining and controlling execution of the synthesis task based on the information set forth by the corresponding specification. For instance, in some embodiments, the corresponding specification includes (i) the identification of respective solvent in one or more solvents (e.g., identification of a first solvent 1920-1 from a library of solvents 1920 available at the molecular foundry 200), (ii) the amount of at least one reactant 1910 in the plurality of reactants 1910, (iii) an x-y address (e.g., address 1930 of FIG. 19) of the well 812 in the first multi-well plate 800-1 including the plurality of wells, (iv) a reaction duration, (v) a reaction temperature (e.g., reaction temperature 1710 of FIG. 17), (vi) a reaction volume, or (vii) a combination thereof. In some embodiments, each synthesis task in the plurality of synthesis tasks includes the corresponding specification that includes (i) the identification of respective solvent 1920 in one or more solvents, (ii) the amount of at least one reactant 1910 in the plurality of reactants 1910, (iii) the x-y address 1930 of the well 812 in the first multi-well plate 800-1 including the plurality of wells, (iv) the reaction duration, (v) the reaction temperature 1710, and (vi) the reaction volume.

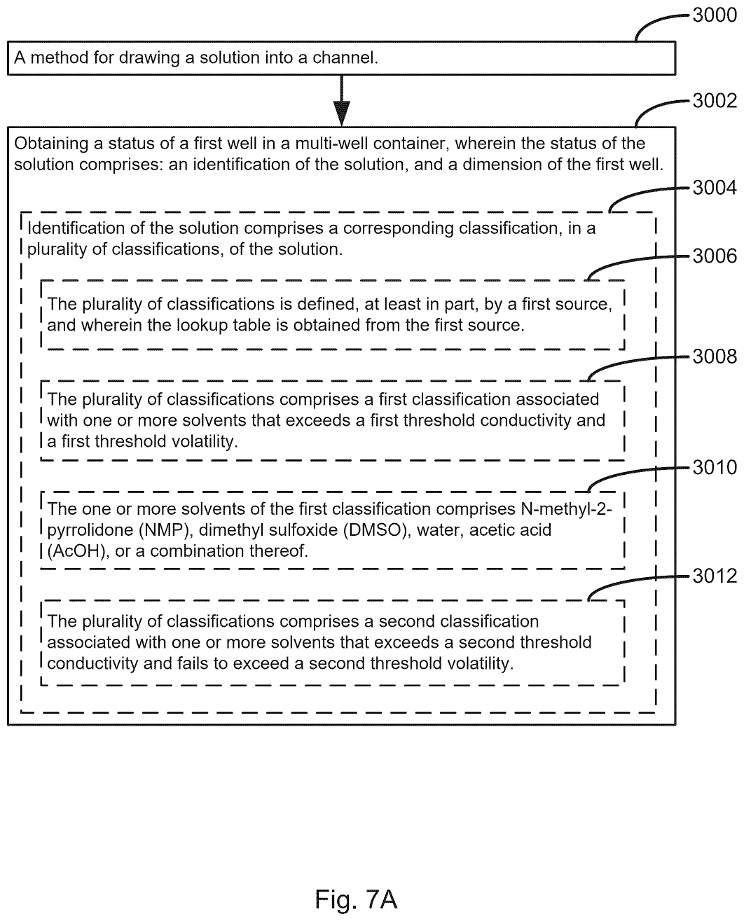

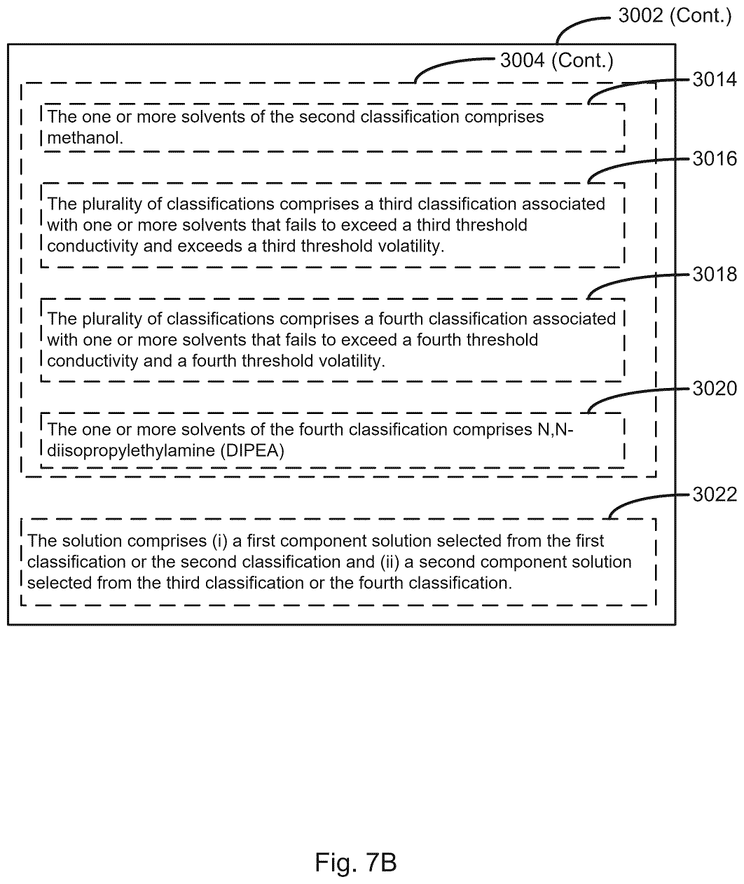

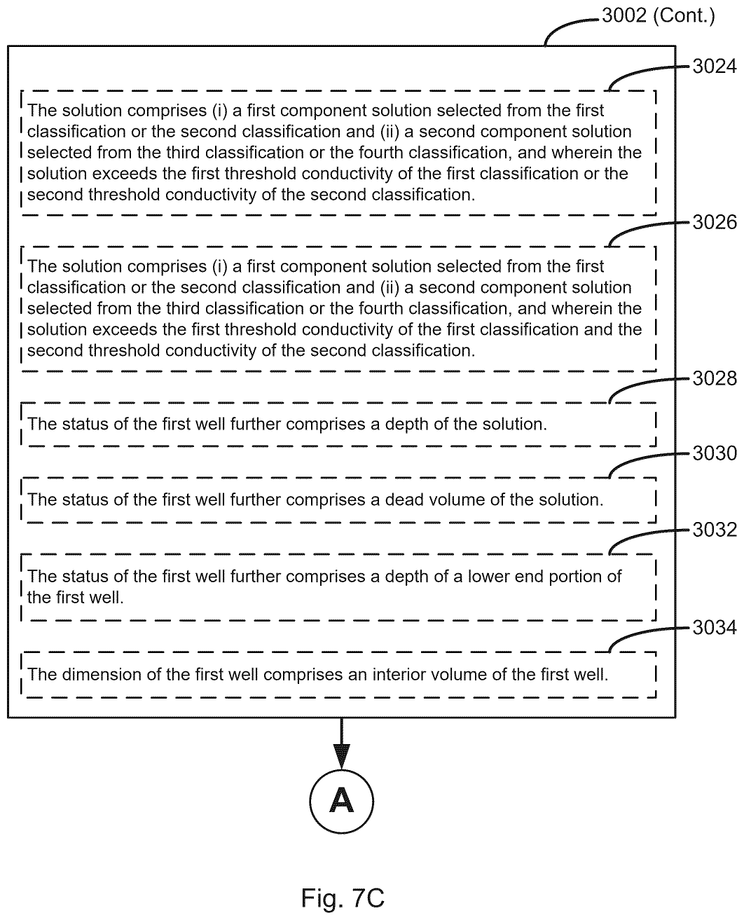

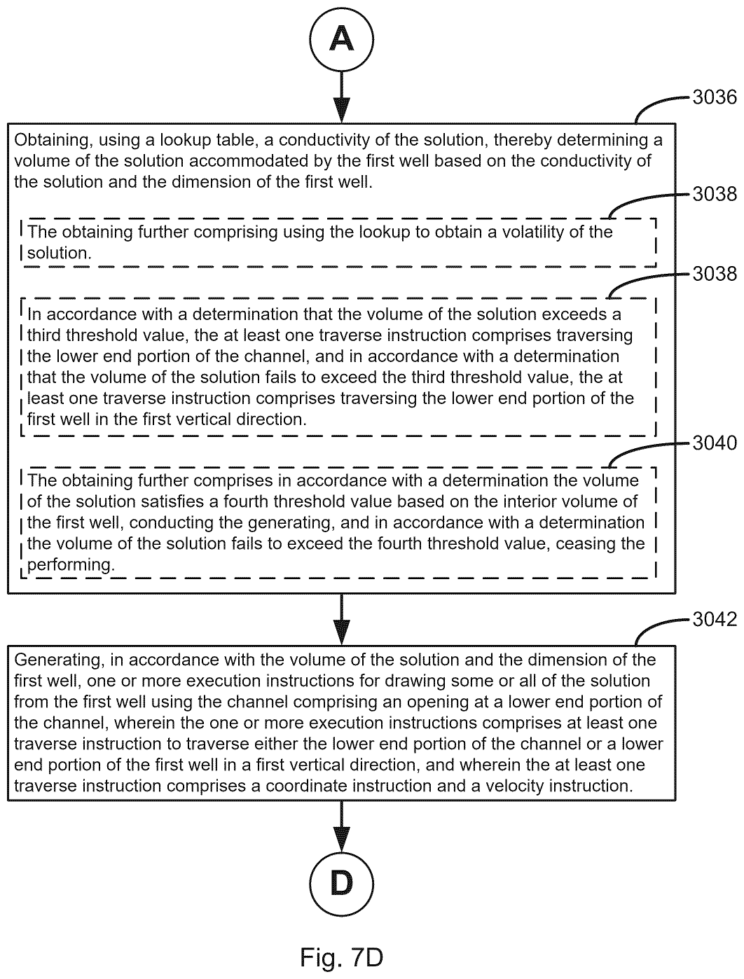

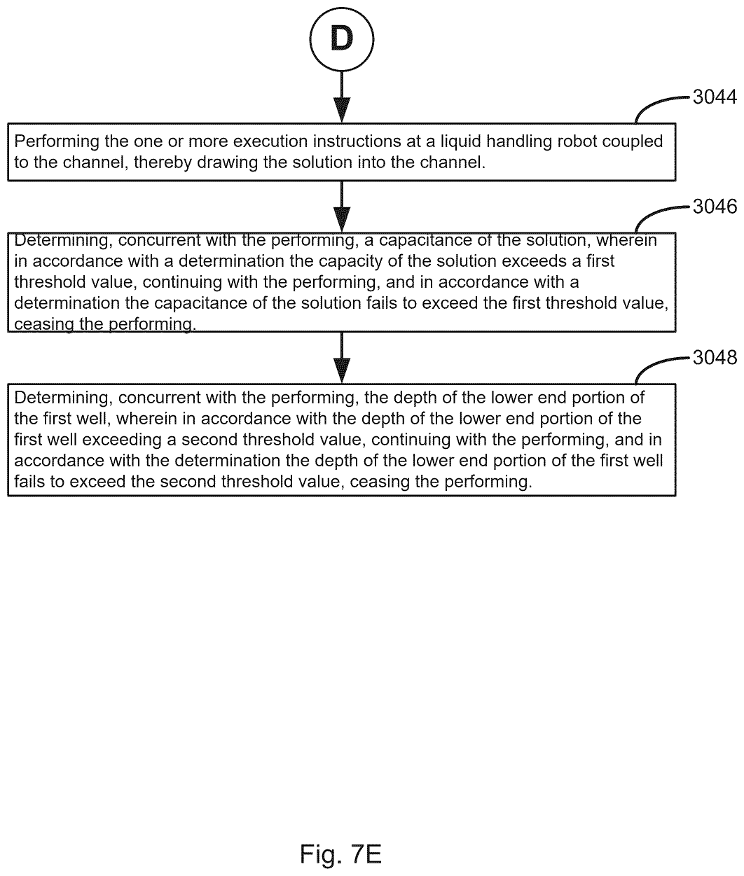

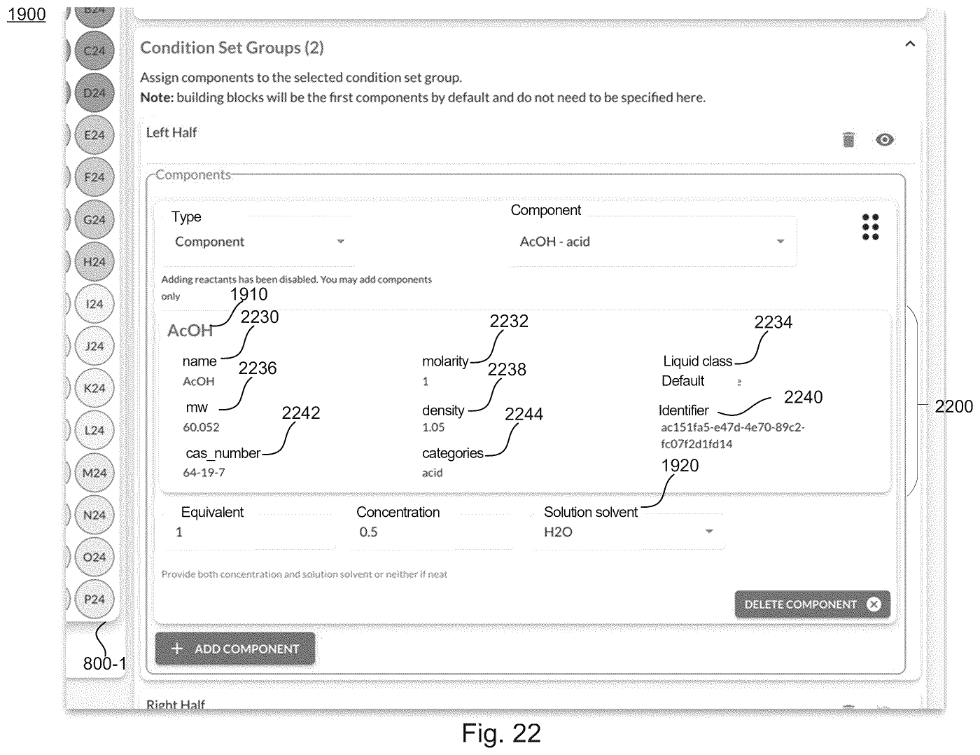

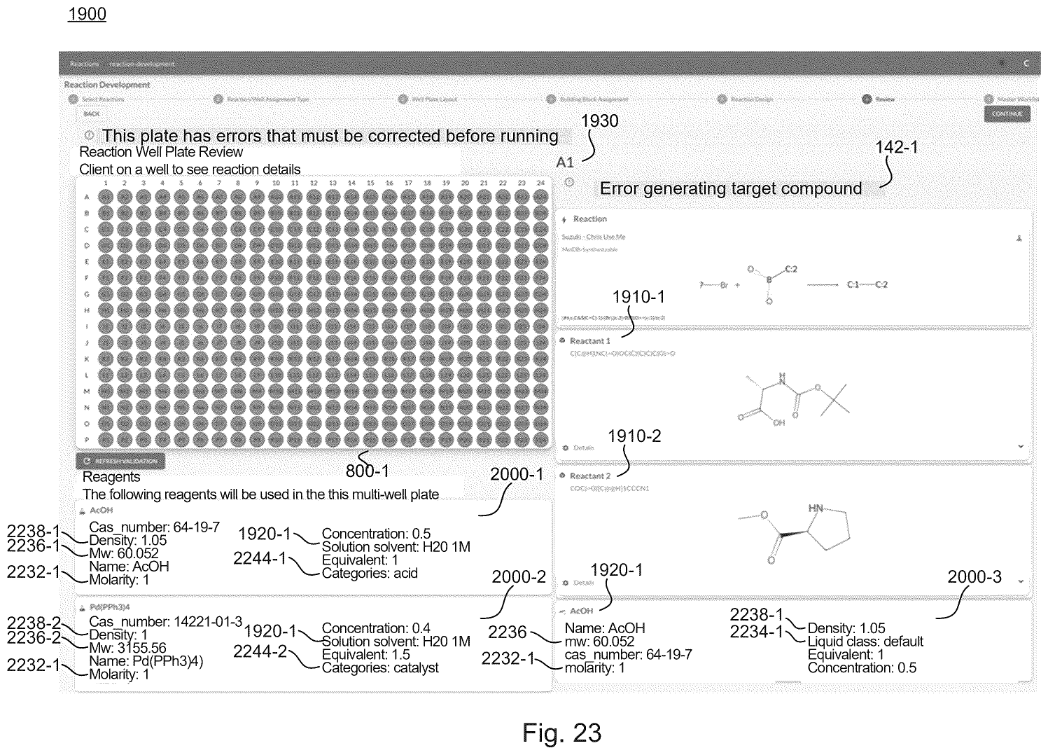

In some embodiments, the identification of respective solvent 1920 in one or more solvents 1920 is based a classification (e.g., liquid classification 2234 of FIG. 22, categories 2244 of FIG. 22, liquid classification 2234 of FIG. 23, categories 2244 of FIG. 23, etc.) of the respective solvent 1920, such as a genus of fluid associated with the respective solvent 1920 and/or a species of fluid associated with the respective solvent 1920, such as a solution 2920 (e.g., block 3002 of FIG. 7A). For instance, referring briefly to FIG. 22, a reactant 1910-1 is associated with a first classification 2234 “Default” liquids and a second classification 2244-2 of catalysts. However, the present disclosure is not limited thereto.

In some embodiments, the amount of at least one reactant 1910 in a plurality of reactants includes a mass of the at least one reactant, a volume of the at least one reactant, a dimensionality of the at least one reactant, or the like. For instance, referring briefly to FIGS. 22 and 23, in some embodiments, the amount of the at least one reactant includes a molarity 2232 of a reactant, a molality of the reactant, a weight percent of the reactant, a volume percent of the reactant, a parts-per-unit (e.g., parts per million (ppm)) of the reactant, a first classification 2234 (e.g., volatility and/or conductivity classification), a second classification 2244 (e.g., a pH classification, an energy classification, etc.), a molecular weight 2236, a density 2238, an identifier or the like.

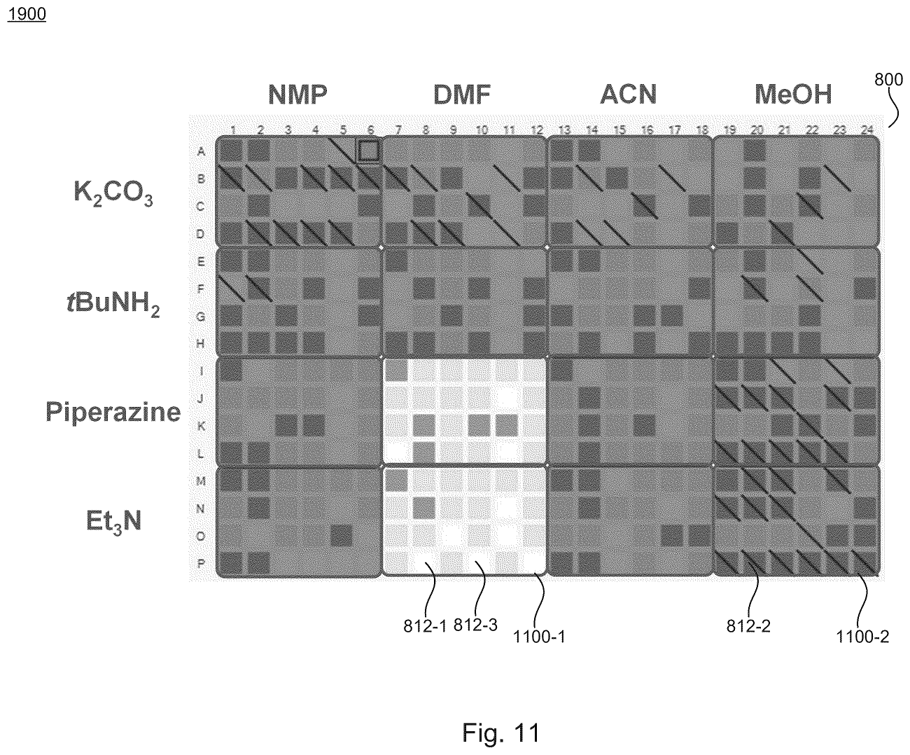

In some embodiments, the x-y address 1930 of the well 812 in a first multi-well plate 800-1 identifies a position of the well 812 on a surface of the first multi-well plate 800-1, such as a horizontal coordinate (e.g., a column 808 of FIG. 8, etc.) and/or a vertical coordinate (e.g., row 806 of FIG. 8) associated with the well 812. For instance, referring briefly to FIG. 11, a first well 812-1 is associated with an X address 808 of 3 (e.g., a third position on a horizontal axis) and a Y address of P 806 (e.g., a sixteenth position on a vertical axis), which yields an x-y address 1930 of 3P for the first well 812-1. As another example, a second well 812-2 of FIG. 11 is associated with an X address 808 of 8 and a Y address 806 of P, which yields an x-y address 1930 of 8P for the second well 812-2. However, the present disclosure is not limited thereto. For instance, in some embodiments, each position of a first axis (e.g., x position) 808 is associated with one or more alphanumeric characters and each position of a second axis (e.g., y position) 806 is associated with one or more alphanumeric characters, which allows for customizing a text string to identify each well 812 of the multi-well plate 800.

Furthermore, in some embodiments, the reaction duration is based on an epoch required to reach equilibrium for the reaction. In some embodiments, the reaction duration is based on an induction epoch of the reaction in which no change is observable, such as a first epoch for a reactant to mix with a solvent. In some embodiments, the reaction duration includes an initiation epoch of the reaction, a dissolution epoch of the reaction, a half-life of one or more reactants, a half-life of one or more solvents, a reaction completion epoch, an equilibrium epoch, a specific conversion reaction epoch, a reaction rate epoch, or a combination thereof.

In some embodiments, the reaction temperature 1710 includes an initial temperature of a reaction, such as a first initial temperature of a solvent and/or a second initial temperature of a reactant. In some embodiments, the reaction temperature 1710 includes a temperature at which a reaction is maintained. In some embodiments, the reaction temperature 1710 includes a peak temperature and/or a minimum temperature, such as a maximum or minimum temperature achieved during the reaction. In some embodiments, the reaction temperature 1710 includes a change in temperature (e.g., delta temperature), such as a different between the initial temperature and the peak temperature and/or a different between the initial temperature and a final temperature. In some embodiments, the reaction temperature 1710 includes a temperature rate, such as a heating rate, a cooling rate, or the like.

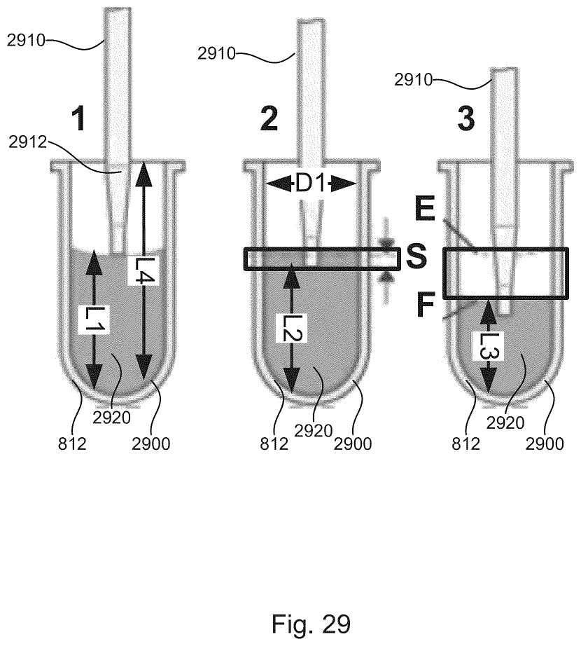

In some embodiments, the reaction volume includes a volume of the solution 2920, a volume of the solvent, a volume of the reactant, a fluid volume of the reaction, a gas volume of the reaction, a vessel volume (e.g., container volume) of the reaction, a total reaction mixture volume, or a combination thereof. For instance, referring briefly to FIG. 29, in some embodiments, the reaction volume includes a volume of the solution 2920, a volume of the well 812 (e.g., vial, container, flask, well, etc.) accommodating the solution 2920, the volume of the well 812 less a volume of the pipettor 2910, or a combination thereof.

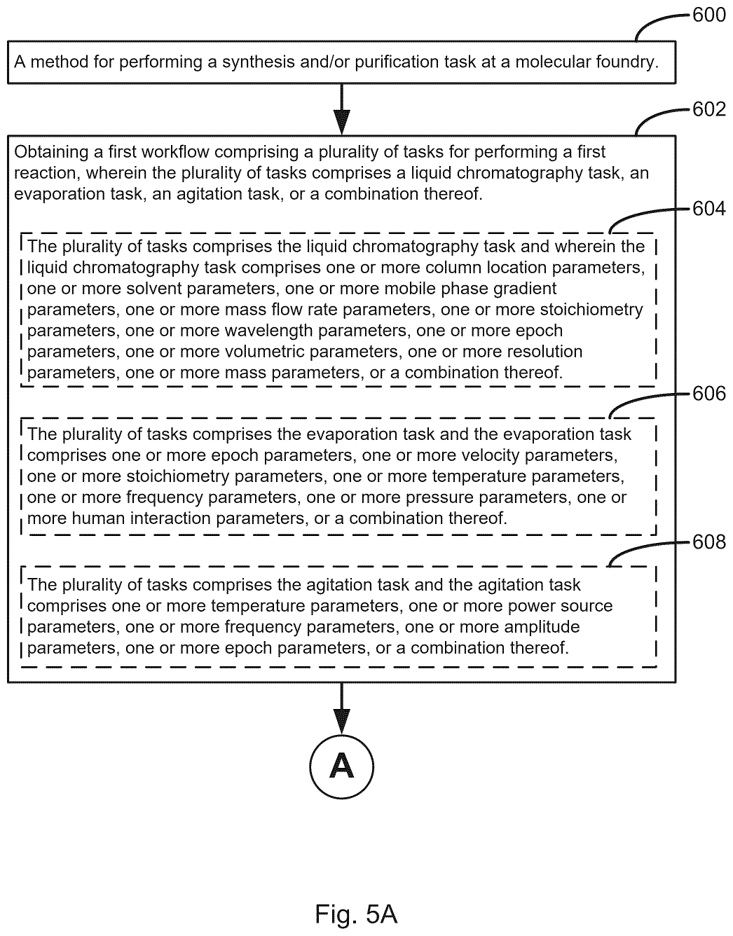

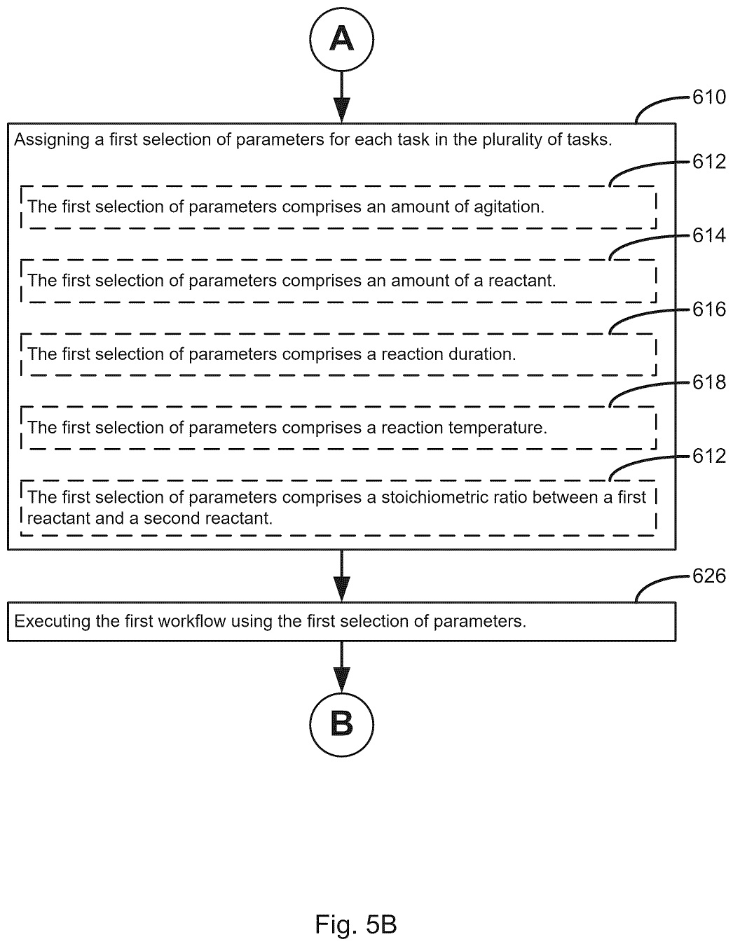

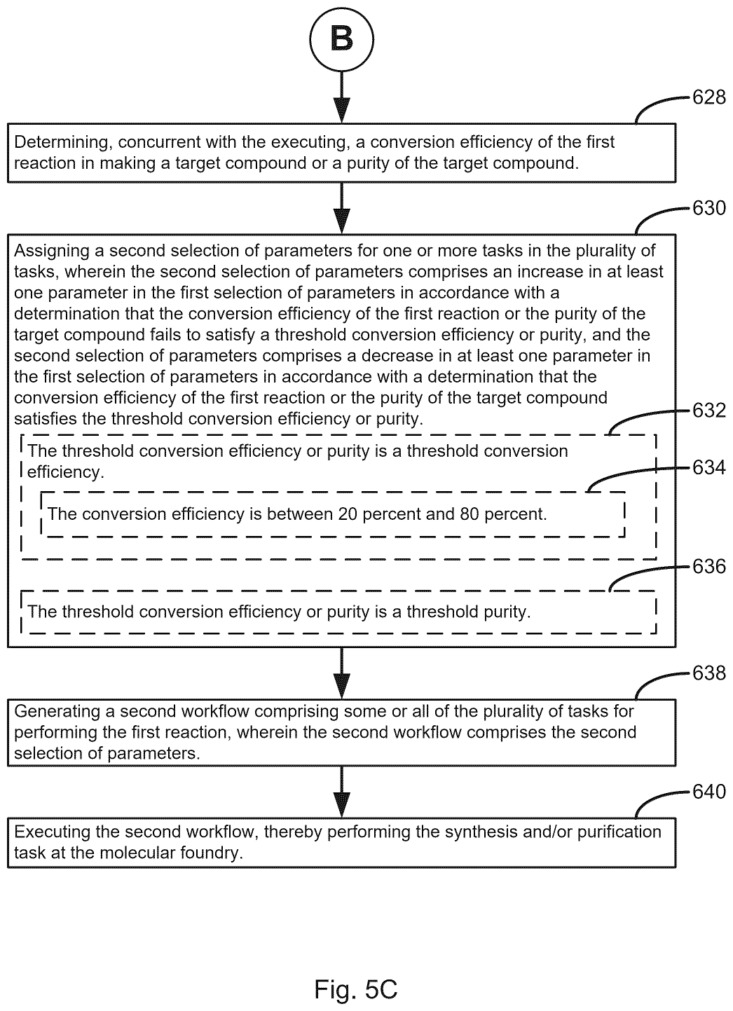

Referring to block 304, in some embodiments, the initial workflow 2400 further includes one or more plating tasks, one or more filtration tasks, one or more dilution tasks, one or more analytical tasks, or any combination thereof, which is utilized to perform a reaction for forming a target compound (e.g., block 602 of FIG. 5A).

Referring to block 306, in some embodiments, the plurality of target compounds consists of organic compounds. In some embodiments, the target compound is a target macromolecule or target macromolecule complex. In some embodiments, the target macromolecule or macromolecule complex comprises one or more active sites to which a respective candidate molecule can bind.

In some embodiments, each respective target compound is a chemical compound. In some embodiments, each respective target compound is a ligand and/or a substrate. In some embodiments, a respective target compound is a large polymer or macromolecule, such as an antibody. In some embodiments, a respective target compound is an organic or inorganic compound.

In some embodiments, an organic compounds is a plurality of reagents of nucleic acid components. For instance, in some embodiments, an organic compound is a plasmid, and the nucleic acid components are predetermined promoters, repressors, stop codon, and exons. In some embodiments, the organic compound is a different predetermined nucleic acid with a different predetermined nucleic acid sequence. In some embodiments, the organic compound is a different predetermined ribonucleic acid (mRNA) with a different predetermined nucleic acid sequence. In some embodiments, the organic compound is a different predetermined deoxyribonucleic acid (DNA) with a different predetermined nucleic acid sequence. In some embodiments, the organic compound is a different predetermined polymer. In some embodiments, the organic compound is a different predetermined peptide. In some embodiments, the organic compound is a different predetermined protein.

In some embodiments, the organic compound comprises a different heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer consists of at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g. (A-B-A-B-B-A-A-A-A-B-B-B)ₙ). Additional examples of copolymers are statistical copolymers in which the sequence of monomer residues in the copolymer follows a statistical rule. If the probability of finding a given type monomer residue at a particular point in the chain is equal to the mole fraction of that monomer residue in the chain, then the polymer may be referred to as a truly random copolymer. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the organic compound a plurality of polymers, where the respective polymers in the plurality of polymers do not all have the same molecular weight. In such embodiments, the polymers in the plurality of polymers fall into a weight range with a corresponding distribution of chain lengths. In some embodiments, the polymer is a branched polymer molecular system comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

Referring to block 308, in some embodiments, the obtaining the initial workflow further includes illuminating a field of view across the first multi-well plate 800-1 with substantially uniform optical characteristics across the field of view, which ensures that each well 812 of the first multi-well plate 800-1 is also uniformly illuminated. For instance, in some embodiments, one or more light sources is utilized to illuminate the field of view across the first multi-well plate 800-1, such as a first light source configured to emit light at a first wavelength and a second light source configured to emit light at a second wavelength different from the first wavelength. In some embodiments, the first and second light sources illuminate light of the same wavelength. From this, the method 300 allows for obtaining optical and/or observable information associated with the multi-well plate without requiring additional compensation for variances in the illumination of the field of view.

Referring to block 310, in some embodiments, the spectral range of light when illuminating the field of view is between 250 nanometers (nm) and 315 nm. For instance, in some embodiments, the spectral range of light is between 250 and 315 nm, 250 and 282 nm, 252 and 313 nm, 252 and 280 nm, 254 and 311 nm, 254 and 278 nm, 256 and 309 nm, 256 and 276 nm, 258 and 307 nm, 258 and 274 nm, 260 and 305 nm, 260 and 272 nm, 263 and 302 nm, 263 and 269 nm, 265 and 300 nm, 265 and 267 nm, 267 and 298 nm, 269 and 296 nm, 271 and 294 nm, 273 and 292 nm, 275 and 290 nm, 277 and 288 nm, 279 and 286 nm, 281 and 284 nm, 282 and 315 nm, 284 and 313 nm, 286 and 311 nm, 288 and 309 nm, 290 and 307 nm, 292 and 305 nm, 295 and 302 nm, or 297 and 300 nm. In some embodiments, the spectral range of light is at least 250 nm, at least 252 nm, at least 254 nm, at least 256 nm, at least 258 nm, at least 260 nm, at least 263 nm, at least 265 nm, at least 267 nm, at least 269 nm, at least 271 nm, at least 272 nm, at least 273 nm, at least 274 nm, at least 275 nm, at least 276 nm, at least 277 nm, at least 278 nm, at least 279 nm, at least 280 nm, at least 281 nm, at least 282 nm, at least 284 nm, at least 286 nm, at least 288 nm, at least 290 nm, at least 292 nm, at least 294 nm, at least 295 nm, at least 296 nm, at least 297 nm, at least 298 nm, at least 300 nm, at least 302 nm, at least 305 nm, at least 307 nm, at least 309 nm, at least 311 nm, at least 313 nm, or at least 315 nm. In some embodiments, the spectral range of light is at most 250 nm, at most 252 nm, at most 254 nm, at most 256 nm, at most 258 nm, at most 260 nm, at most 263 nm, at most 265 nm, at most 267 nm, at most 269 nm, at most 271 nm, at most 272 nm, at most 273 nm, at most 274 nm, at most 275 nm, at most 276 nm, at most 277 nm, at most 278 nm, at most 279 nm, at most 280 nm, at most 281 nm, at most 282 nm, at most 284 nm, at most 286 nm, at most 288 nm, at most 290 nm, at most 292 nm, at most 294 nm, at most 295 nm, at most 296 nm, at most 297 nm, at most 298 nm, at most 300 nm, at most 302 nm, at most 305 nm, at most 307 nm, at most 309 nm, at most 311 nm, at most 313 nm, or at most 315 nm.

Referring to block 312, in some embodiments, the multi and well plate includes between 24 wells and 400 wells, between 24 wells and 384 wells, between 50 wells and 400 wells, between 50 wells and 350 wells, 50 and 300 wells, 50 and 175 wells, 58 and 292 wells, 58 and 167 wells, 66 and 284 wells, 66 and 159 wells, 74 and 276 wells, 74 and 151 wells, 82 and 268 wells, 82 and 143 wells, 90 and 260 wells, 90 and 135 wells, 98 and 252 wells, 98 and 127 wells, 106 and 244 wells, 106 and 119 wells, 115 and 235 wells, 123 and 227 wells, 131 and 219 wells, 139 and 211 wells, 147 and 203 wells, 155 and 195 wells, 163 and 187 wells, 171 and 179 wells, 175 and 300 wells, 183 and 292 wells, 191 and 284 wells, 199 and 276 wells, 207 and 268 wells, 215 and 260 wells, 223 and 252 wells, or 231 and 244 wells. In some embodiments, the multi and well plate includes at least 24 wells, at least 50 wells, at least 58 wells, at least 66 wells, at least 74 wells, at least 82 wells, at least 90 wells, at least 98 wells, at least 106 wells, at least 115 wells, at least 119 wells, at least 123 wells, at least 127 wells, at least 131 wells, at least 135 wells, at least 139 wells, at least 143 wells, at least 147 wells, at least 151 wells, at least 155 wells, at least 159 wells, at least 163 wells, at least 167 wells, at least 171 wells, at least 175 wells, at least 179 wells, at least 183 wells, at least 187 wells, at least 191 wells, at least 195 wells, at least 199 wells, at least 203 wells, at least 207 wells, at least 211 wells, at least 215 wells, at least 219 wells, at least 223 wells, at least 227 wells, at least 231 wells, at least 235 wells, at least 244 wells, at least 252 wells, at least 260 wells, at least 268 wells, at least 276 wells, at least 284 wells, at least 292 wells, at least 300 wells, at least 350 wells, at least 384 wells, or at least 400 wells. In some embodiments, the multi-well plate at most 24 wells, at most 50 wells, at most 58 wells, at most 66 wells, at most 74 wells, at most 82 wells, at most 90 wells, at most 98 wells, at most 106 wells, at most 115 wells, at most 119 wells, at most 123 wells, at most 127 wells, at most 131 wells, at most 135 wells, at most 139 wells, at most 143 wells, at most 147 wells, at most 151 wells, at most 155 wells, at most 159 wells, at most 163 wells, at most 167 wells, at most 171 wells, at most 175 wells, at most 179 wells, at most 183 wells, at most 187 wells, at most 191 wells, at most 195 wells, at most 199 wells, at most 203 wells, at most 207 wells, at most 211 wells, at most 215 wells, at most 219 wells, at most 223 wells, at most 227 wells, at most 231 wells, at most 235 wells, at most 244 wells, at most 252 wells, at most 260 wells, at most 268 wells, at most 276 wells, at most 284 wells, at most 292 wells, at most 300 wells, at most 350 wells, at most 384 wells, or at least most 400 wells.

Referring to block 314, in some embodiments, the reaction duration is between 1 second and two days. For instance, in some embodiments, the reaction duration is 1 and 3600 seconds, 1 and 1800 seconds, 117 and 3484 seconds, 117 and 1684 seconds, 233 and 3368 seconds, 233 and 1568 seconds, 349 and 3252 seconds, 349 and 1452 seconds, 465 and 3136 seconds, 465 and 1336 seconds, 581 and 3020 seconds, 581 and 1220 seconds, 698 and 2903 seconds, 698 and 1103 seconds, 814 and 2787 seconds, 814 and 987 seconds, 930 and 2671 seconds, 1046 and 2555 seconds, 1162 and 2439 seconds, 1278 and 2323 seconds, 1394 and 2207 seconds, 1510 and 2091 seconds, 1626 and 1975 seconds, 1742 and 1859 seconds, 1800 and 3600 seconds, 1916 and 3484 seconds, 2032 and 3368 seconds, 2148 and 3252 seconds, 2264 and 3136 seconds, 2380 and 3020 seconds, 2497 and 2903 seconds, 2613 and 2787 seconds, 1 and 48 hours, 1 and 24 hours, 2 and 46 hours, 2 and 22 hours, 3 and 45 hours, 3 and 21 hours, 5 and 43 hours, 5 and 19 hours, 6 and 42 hours, 6 and 18 hours, 8 and 40 hours, 8 and 16 hours, 9 and 39 hours, 9 and 15 hours, 11 and 37 hours, 11 and 13 hours, 12 and 36 hours, 14 and 34 hours, 15 and 33 hours, 17 and 31 hours, 19 and 29 hours, 20 and 28 hours, 22 and 26 hours, 23 and 25 hours, 24 and 48 hours, 26 and 46 hours, 27 and 45 hours, 29 and 43 hours, 30 and 42 hours, 32 and 40 hours, or 33 and 39 hours.

In some embodiments, the reaction duration is at least 1 seconds, at least 117 seconds, at least 233 seconds, at least 349 seconds, at least 465 seconds, at least 581 seconds, at least 698 seconds, at least 814 seconds, at least 930 seconds, at least 987 seconds, at least 1046 seconds, at least 1103 seconds, at least 1162 seconds, at least 1220 seconds, at least 1278 seconds, at least 1336 seconds, at least 1394 seconds, at least 1452 seconds, at least 1510 seconds, at least 1568 seconds, at least 1626 seconds, at least 1684 seconds, at least 1742 seconds, at least 1800 seconds, at least 1859 seconds, at least 1916 seconds, at least 1975 seconds, at least 2032 seconds, at least 2091 seconds, at least 2148 seconds, at least 2207 seconds, at least 2264 seconds, at least 2323 seconds, at least 2380 seconds, at least 2439 seconds, at least 2497 seconds, at least 2555 seconds, at least 2613 seconds, at least 2671 seconds, at least 2787 seconds, at least 2903 seconds, at least 3020 seconds, at least 3136 seconds, at least 3252 seconds, at least 3368 seconds, at least 3484 seconds, at least 3600 seconds, at least 0 hours, at least 2 hours, at least 3 hours, at least 5 hours, at least 6 hours, at least 8 hours, at least 9 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 39 hours, at least 40 hours, at least 42 hours, at least 43 hours, at least 45 hours, at least 46 hours, or at least 48 hours.

In some embodiments, the reaction duration at most 1 seconds, at most 117 seconds, at most 233 seconds, at most 349 seconds, at most 465 seconds, at most 581 seconds, at most 698 seconds, at most 814 seconds, at most 930 seconds, at most 987 seconds, at most 1046 seconds, at most 1103 seconds, at most 1162 seconds, at most 1220 seconds, at most 1278 seconds, at most 1336 seconds, at most 1394 seconds, at most 1452 seconds, at most 1510 seconds, at most 1568 seconds, at most 1626 seconds, at most 1684 seconds, at most 1742 seconds, at most 1800 seconds, at most 1859 seconds, at most 1916 seconds, at most 1975 seconds, at most 2032 seconds, at most 2091 seconds, at most 2148 seconds, at most 2207 seconds, at most 2264 seconds, at most 2323 seconds, at most 2380 seconds, at most 2439 seconds, at most 2497 seconds, at most 2555 seconds, at most 2613 seconds, at most 2671 seconds, at most 2787 seconds, at most 2903 seconds, at most 3020 seconds, at most 3136 seconds, at most 3252 seconds, at most 3368 seconds, at most 3484 seconds, at most 3600 seconds, at most 0 hours, at most 2 hours, at most 3 hours, at most 5 hours, at most 6 hours, at most 8 hours, at most 9 hours, at most 11 hours, at most 12 hours, at most 13 hours, at most 14 hours, at most 15 hours, at most 16 hours, at most 17 hours, at most 18 hours, at most 19 hours, at most 20 hours, at most 21 hours, at most 22 hours, at most 23 hours, at most 24 hours, at most 25 hours, at most 26 hours, at most 27 hours, at most 28 hours, at most 29 hours, at most 30 hours, at most 31 hours, at most 32 hours, at most 33 hours, at most 34 hours, at most 35 hours, at most 36 hours, at most 37 hours, at most 39 hours, at most 40 hours, at most 42 hours, at most 43 hours, at most 45 hours, at most 46 hours, at most 48 hours.

Referring to block 336, in some embodiments, the reaction temperature 1710 is between 0° C. and 99° C. For instance, in some embodiments, the reaction temperature 1710 is between 0 and 99° C. 0 and 50° C., 3 and 96° C., 3 and 47° C., 6 and 93° C., 6 and 44° C., 10 and 89° C., 10 and 40° C., 13 and 86° C., 13 and 37° C., 16 and 83° C., 16 and 34° C., 19 and 80° C., 19 and 31° C., 22 and 77° C., 22 and 28° C., 26 and 73° C., 29 and 70° C., 32 and 67° C., 35 and 64° C., 38 and 61° C., 42 and 57° C., 45 and 54° C., 48 and 51° C., 50 and 99° C., 53 and 96° C., 56 and 93° C., 60 and 89° C., 63 and 86° C., 66 and 83° C., 69 and 80° C., or 72 and 77° C. In some embodiments, the reaction temperature 1710 is at least 0° C., at least 3° C., at least 6° C., at least 10° C., at least 13° C., at least 16° C., at least 19° C., at least 22° C., at least 26° C., at least 28° C., at least 29° C., at least 31° C., at least 32° C., at least 34° C., at least 35° C., at least 37° C., at least 38° C., at least 40° C., at least 42° C., at least 44° C., at least 45° C., at least 47° C., at least 48° C., at least 50° C., at least 51° C., at least 53° C., at least 54° C., at least 56° C., at least 57° C., at least 60° C., at least 61° C., at least 63° C., at least 64° C., at least 66° C., at least 67° C., at least 69° C., at least 70° C., at least 72° C., at least 73° C., at least 77° C., at least 80° C., at least 83° C., at least 86° C., at least 89° C., at least 93° C., at least 96° C., at least 99° C. In some embodiments, the reaction temperature 1710 is at most 0° C., at most 3° C., at most 6° C., at most 10° C., at most 13° C., at most 16° C., at most 19° C., at most 22° C., at most 26° C., at most 28° C., at most 29° C., at most 31° C., at most 32° C., at most 34° C., at most 35° C., at most 37° C., at most 38° C., at most 40° C., at most 42° C., at most 44° C., at most 45° C., at most 47° C., at most 48° C., at most 50° C., at most 51° C., at most 53° C., at most 54° C., at most 56° C., at most 57° C., at most 60° C., at most 61° C., at most 63° C., at most 64° C., at most 66° C., at most 67° C., at most 69° C., at most 70° C., at most 72° C., at most 73° C., at most 77° C., at most 80° C., at most 83° C., at most 86° C., at most 89° C., at most 93° C., at most 96° C., at most 99° C. In some embodiments, the reaction temperature 1710 is ambient temperature (e.g., between 2° and 22° C.).

Referring to block 318 of FIG. 3B, in some embodiments, the respective solvent 1920 in a corresponding specification is water, dimethyl sulfoxide (DMSO), acetonitrile, dimethyl ether, chloroform, hexanes, toluene, dichloromethane, N-Methyl-2-pyrrolidone (NMP), methanol, or a combination thereof. For instance, in some embodiments, the respective solvent 1920 in the corresponding specification includes one or more liquids used to dissolve various organic compounds, such as a non-polar solvent, a slightly polar solvent, an aprotic solvent, a protic solvent, or the like. As a non-limiting example, in some embodiments, the respective solvent 1920 in the corresponding specification includes hexanes, toluene, benzene, diethyl ether, chloroform, dichloromethane, acetone, acetonitrile, dimethyl sulfoxide, dimethylformamide, tetrahydrofuran, ethanol, methanol, water, formic acid, pyridine, or the like.

Referring to block 320, in some embodiments, the reaction volume is between 10 μL and 10000 μL. For instance, in some embodiments, the reaction volume is between 10 and 10000 μL, 10 and 5005 μL, 332 and 9678 μL, 332 and 4683 μL, 655 and 9355 μL, 655 and 4360 μL, 977 and 9033 μL, 977 and 4038 μL, 1299 and 8711 μL, 1299 and 3716 μL, 1621 and 8389 μL, 1621 and 3394 μL, 1944 and 8066 μL, 1944 and 3071 μL, 2266 and 7744 μL, 2266 and 2749 μL, 2588 and 7422 μL, 2910 and 7100 μL, 3233 and 6777 μL, 3555 and 6455 μL, 3877 and 6133 μL, 4199 and 5811 μL, 4522 and 5488 μL, 4844 and 5166 μL, 5005 and 10000 μL, 5327 and 9678 μL, 5650 and 9355 μL, 5972 and 9033 μL, 6294 and 8711 μL, 6616 and 8389 μL, 6939 and 8066 μL, 7261 and 7744 μL, 50 and 2000 μL, 50 and 1025 μL, 113 and 1937 μL, 113 and 962 μL, 176 and 1874 μL, 176 and 899 μL, 239 and 1811 μL, 239 and 836 μL, 302 and 1748 μL, 302 and 773 μL, 365 and 1685 μL, 365 and 710 μL, 427 and 1623 μL, 427 and 648 μL, 490 and 1560 μL, 490 and 585 μL, 553 and 1497 μL, 616 and 1434 μL, 679 and 1371 μL, 742 and 1308 μL, 805 and 1245 μL, 868 and 1182 μL, 931 and 1119 μL, 994 and 1056 μL, 1025 and 2000 μL, 1088 and 1937 μL, 1151 and 1874 μL, 1214 and 1811 μL, 1277 and 1748 μL, 1340 and 1685 μL, 1402 and 1623 μL, or 1465 and 1560 μL.

In some embodiments, the reaction volume is at least 50 μL, at least 113 μL, at least 176 μL, at least 239 μL, at least 302 μL, at least 365 μL, at least 427 μL, at least 490 μL, at least 553 μL, at least 585 μL, at least 616 μL, at least 648 μL, at least 679 μL, at least 710 μL, at least 742 μL, at least 773 μL, at least 805 μL, at least 836 μL, at least 868 μL, at least 899 μL, at least 931 μL, at least 962 μL, at least 994 μL, at least 1025 μL, at least 1056 μL, at least 1088 μL, at least 1119 μL, at least 1151 μL, at least 1182 μL, at least 1214 μL, at least 1245 μL, at least 1277 μL, at least 1308 μL, at least 1340 μL, at least 1371 μL, at least 1402 μL, at least 1434 μL, at least 1465 μL, at least 1497 μL, at least 1560 μL, at least 1623 μL, at least 1685 μL, at least 1748 μL, at least 1811 μL, at least 1874 μL, at least 1937 μL, or at least 2000 μL. In some embodiments, the reaction volume is at least 10 μL, at least 332 μL, at least 655 μL, at least 977 μL, at least 1299 μL, at least 1621 μL, at least 1944 μL, at least 2266 μL, at least 2588 μL, at least 2749 μL, at least 2910 μL, at least 3071 μL, at least 3233 μL, at least 3394 μL, at least 3555 μL, at least 3716 μL, at least 3877 μL, at least 4038 μL, at least 4199 μL, at least 4360 μL, at least 4522 μL, at least 4683 μL, at least 4844 μL, at least 5005 μL, at least 5166 μL, at least 5327 μL, at least 5488 μL, at least 5650 μL, at least 5811 μL, at least 5972 μL, at least 6133 μL, at least 6294 μL, at least 6455 μL, at least 6616 μL, at least 6777 μL, at least 6939 μL, at least 7100 μL, at least 7261 μL, at least 7422 μL, at least 7744 μL, at least 8066 μL, at least 8389 μL, at least 8711 μL, at least 9033 μL, at least 9355 μL, at least 9678 μL, or at least 10000 μL. In some embodiments, the reaction volume is at most 50 μL, at most 113 μL, at most 176 μL, at most 239 μL, at most 302 μL, at most 365 μL, at most 427 μL, at most 490 μL, at most 553 μL, at most 585 μL, at most 616 μL, at most 648 μL, at most 679 μL, at most 710 μL, at most 742 μL, at most 773 μL, at most 805 μL, at most 836 μL, at most 868 μL, at most 899 μL, at most 931 μL, at most 962 μL, at most 994 μL, at most 1025 μL, at most 1056 μL, at most 1088 μL, at most 1119 μL, at most 1151 μL, at most 1182 μL, at most 1214 μL, at most 1245 μL, at most 1277 μL, at most 1308 μL, at most 1340 μL, at most 1371 μL, at most 1402 μL, at most 1434 μL, at most 1465 μL, at most 1497 μL, at most 1560 μL, at most 1623 μL, at most 1685 μL, at most 1748 μL, at most 1811 μL, at most 1874 μL, at most 1937 μL, or at most 2000 μL. In some embodiments, the reaction volume is at most 10 μL, at most 332 μL, at most 655 μL, at most 977 μL, at most 1299 μL, at most 1621 μL, at most 1944 μL, at most 2266 μL, at most 2588 μL, at most 2749 μL, at most 2910 μL, at most 3071 μL, at most 3233 μL, at most 3394 μL, at most 3555 μL, at most 3716 μL, at most 3877 μL, at most 4038 μL, at most 4199 μL, at most 4360 μL, at most 4522 μL, at most 4683 μL, at most 4844 μL, at most 5005 μL, at most 5166 μL, at most 5327 μL, at most 5488 μL, at most 5650 μL, at most 5811 μL, at most 5972 μL, at most 6133 μL, at most 6294 μL, at most 6455 μL, at most 6616 μL, at most 6777 μL, at most 6939 μL, at most 7100 μL, at most 7261 μL, at most 7422 μL, at most 7744 μL, at most 8066 μL, at most 8389 μL, at most 8711 μL, at most 9033 μL, at most 9355 μL, at most 9678 μL, or at most 10000 μL.

Referring to blocks 322-326, in some embodiments, the plurality of target compounds includes 10 or more compounds, 100 or more compounds, or 1,000 or more compounds. In some embodiments, the plurality of target compounds comprises at least 2 or more, at least 4 or more, at least 5 or more, at least 10 or more, at least 20 or more, at least 50 or more, at least 100 or more, at least 500 or more, at least 1000 or more, at least 10,000, or at least 100,000 compounds. In some embodiments, the plurality of target compounds comprises 100 or more, 500 or more, 1000 or more, 2000 or more or 10,000 or more compounds. In some embodiments, the plurality of compounds comprises no more than 1×10⁶, no more than 100,000, no more than 10,000, no more than 1000, no more than 100, or no more than 50 compounds. In some embodiments, the plurality of target compounds consists of from 2 to 20, from 10 to 100, from 50 to 1000, from 500 to 10,000, from 2000 to 500,000, or from 100,000 to 1×10⁶ compounds.

Referring to blocks 328-334, in some embodiments, the plurality of synthesis tasks includes 20 or more synthesis tasks. In some embodiments, the plurality of synthesis tasks includes 50 or more synthesis tasks. In some embodiments, the plurality of synthesis tasks includes 100 or more synthesis tasks. In some embodiments, the plurality of synthesis tasks includes 1000 or more synthesis tasks. For instance, in some embodiments, 20 and 1000 synthesis tasks, 20 and 510 synthesis tasks, 52 and 968 synthesis tasks, 52 and 478 synthesis tasks, 83 and 937 synthesis tasks, 83 and 447 synthesis tasks, 115 and 905 synthesis tasks, 115 and 415 synthesis tasks, 146 and 874 synthesis tasks, 146 and 384 synthesis tasks, 178 and 842 synthesis tasks, 178 and 352 synthesis tasks, 210 and 810 synthesis tasks, 210 and 320 synthesis tasks, 241 and 779 synthesis tasks, 241 and 289 synthesis tasks, 273 and 747 synthesis tasks, 305 and 715 synthesis tasks, 336 and 684 synthesis tasks, 368 and 652 synthesis tasks, 399 and 621 synthesis tasks, 431 and 589 synthesis tasks, 463 and 557 synthesis tasks, 494 and 526 synthesis tasks, 510 and 1000 synthesis tasks, 542 and 968 synthesis tasks, 573 and 937 synthesis tasks, 605 and 905 synthesis tasks, 636 and 874 synthesis tasks, 668 and 842 synthesis tasks, 700 and 810 synthesis tasks, or 731 and 779 synthesis tasks. In some embodiments, the plurality of synthesis tasks includes at least 20 synthesis tasks, at least 52 synthesis tasks, at least 83 synthesis tasks, at least 115 synthesis tasks, at least 146 synthesis tasks, at least 178 synthesis tasks, at least 210 synthesis tasks, at least 241 synthesis tasks, at least 273 synthesis tasks, at least 289 synthesis tasks, at least 305 synthesis tasks, at least 320 synthesis tasks, at least 336 synthesis tasks, at least 352 synthesis tasks, at least 368 synthesis tasks, at least 384 synthesis tasks, at least 399 synthesis tasks, at least 415 synthesis tasks, at least 431 synthesis tasks, at least 447 synthesis tasks, at least 463 synthesis tasks, at least 478 synthesis tasks, at least 494 synthesis tasks, at least 510 synthesis tasks, at least 526 synthesis tasks, at least 542 synthesis tasks, at least 557 synthesis tasks, at least 573 synthesis tasks, at least 589 synthesis tasks, at least 605 synthesis tasks, at least 621 synthesis tasks, at least 636 synthesis tasks, at least 652 synthesis tasks, at least 668 synthesis tasks, at least 684 synthesis tasks, at least 700 synthesis tasks, at least 715 synthesis tasks, at least 731 synthesis tasks, at least 747 synthesis tasks, at least 779 synthesis tasks, at least 810 synthesis tasks, at least 842 synthesis tasks, at least 874 synthesis tasks, at least 905 synthesis tasks, at least 937 synthesis tasks, at least 968 synthesis tasks, or at least 1000 synthesis tasks. In some embodiments, the plurality of synthesis tasks includes at most 20 synthesis tasks, at most 52 synthesis tasks, at most 83 synthesis tasks, at most 115 synthesis tasks, at most 146 synthesis tasks, at most 178 synthesis tasks, at most 210 synthesis tasks, at most 241 synthesis tasks, at most 273 synthesis tasks, at most 289 synthesis tasks, at most 305 synthesis tasks, at most 320 synthesis tasks, at most 336 synthesis tasks, at most 352 synthesis tasks, at most 368 synthesis tasks, at most 384 synthesis tasks, at most 399 synthesis tasks, at most 415 synthesis tasks, at most 431 synthesis tasks, at most 447 synthesis tasks, at most 463 synthesis tasks, at most 478 synthesis tasks, at most 494 synthesis tasks, at most 510 synthesis tasks, at most 526 synthesis tasks, at most 542 synthesis tasks, at most 557 synthesis tasks, at most 573 synthesis tasks, at most 589 synthesis tasks, at most 605 synthesis tasks, at most 621 synthesis tasks, at most 636 synthesis tasks, at most 652 synthesis tasks, at most 668 synthesis tasks, at most 684 synthesis tasks, at most 700 synthesis tasks, at most 715 synthesis tasks, at most 731 synthesis tasks, at most 747 synthesis tasks, at most 779 synthesis tasks, at most 810 synthesis tasks, at most 842 synthesis tasks, at most 874 synthesis tasks, at most 905 synthesis tasks, at most 937 synthesis tasks, at most 968 synthesis tasks, at most 1000 synthesis tasks. Accordingly, in some such embodiments, the plurality of synthesis tasks is of such numerosity and complexity that the method 300 cannot be performed by a human mind or pen and paper. Said otherwise, in some such embodiments, the method 300 requires utilization of the computing system 100 due to the computational burden and complexity associated with the plurality of synthesis tasks.

Referring to block 336, in some embodiments, a first synthesis task in the plurality of synthesis task includes a first plurality of step instructions for controlling a first pipettor 2900 in fluid communication with a first reactant, in the one or more reactants specified by the first synthesis task, that is dissolved in a respective solvent specified by the first synthesis task, and a plurality of x-y plate instructions for causing the x-y address of a first well 812-1 in the first multi-well plate 800-1 specified by the first synthesis task to be in fluid communication with the first pipettor 2900.

For instance, in some embodiments, each respective well 812 includes the x-y address of the respective instrument and one or more parameters associated with the respective well, such as one or more components accommodated by the well 812, a volume of the well 812, and the like. In some embodiments, instrument execution instruction(s) are at least parameter sets used in programming of process steps and workflows. In some embodiments, the instrument execution instructions sets are the configurations of machines and process conditions. In some embodiments, such machine configurations and process conditions include, but are not limited to, adjusting rotations per minute (RPM) of a machine, changing a status of a machine from or to ON and OFF, and the like. In some embodiments, the instrument execution instructions is a logical dependency of steps in a workflow that defines the procedures that wells 812 are processed (e.g., block 362 of FIG. 3D). As a non-limiting example, an instrument executable instruction(s) can include, but are not limited to:
run Horizontal Traverse Pipette 1 with Parameter Set 1 to Well P3 from Well P4
run Vertical Traverse Pipette 1 with Parameter Set 2 for Well P3
run Process module 2 with Parameter Set 3 for Well P3

In some embodiments, step instructions can either be interpreted as specific value or coordinate instructions such as:
Parameter Set 1 through n
Sample 1 through n
Threshold 1 through n

Additionally, step instructions can be interpreted as dependencies in the process such as:
execute 2) after 1)
execute 3) after 2)
execute 4) after logic decision 3)
execute 6) after logic decision 3

As a non-limiting example, for a first apparatus, in some embodiments, the instructions can be interpreted as:
Time (min): parameter 1, value 1 (e.g., 20 minute epoch duration)
Vortex (RPM): parameter 2, value 2 (e.g., 200 RPM)
Vortex acceleration: parameter 3, values 3-5 (e.g., 5 G)
Temperature (° C.): parameter 4, value 4 (e.g., values 3-5 is each 40° C.)
Pump mode: State 1, value 5 (e.g., ON State)
Pump pressure (mbar): parameter 5, value 6 (e.g., 14 mbar)
Pump frequency (Hz): parameter 7, value 7 (35 Hz)

Referring to block 338 of FIG. 3C, in some embodiments, the first synthesis task further includes: a set of instructions to cause the first pipettor 2900 to switch from being in fluid communication with the first reactant, to being in fluid communication with a second reactant, in the one or more reactants specified by the first synthesis task, that is dissolved in a respective solvent specified by the first synthesis task, and a plurality of step instructions for causing the first pipettor to dispense an amount of the second reactant that is dissolved in a respective solvent, specified by the first synthesis task specified by the first synthesis task, into the first well.

Referring to block 340, in some embodiments, each target compound in the plurality of target compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. In some embodiments, each target compound in the plurality of target compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety for all purposes. In some embodiments, each target compound in the plurality of target compounds satisfies one or more criteria in addition to Lipinski's Rule of Five. For example, in some embodiments, each compound in the plurality of compounds satisfies has five or fewer aromatic rings, four or fewer aromatic rings, three or fewer aromatic rings, or two or fewer aromatic rings.

Referring to blocks 342-350, in some embodiments, each target compound in the plurality of target compounds is organic compound having a molecular weight of less than 500 Daltons. In some embodiments, each target compound in the plurality of target compounds is an organic compound having a molecular weight of less than 1000 Daltons. In some embodiments, each target compound in the plurality of target compounds is an organic compound having a molecular weight of less than 2000 Daltons. In some embodiments, each target compound in the plurality of target compounds is an organic compound having a molecular weight of less than 4000 Daltons, less than 6000 Daltons, less than 8000 Daltons, less than 10000 Daltons, or less than 20000 Daltons. In some embodiments, each target compound in the plurality of target compounds is organic compound having a molecular weight of between 300 Daltons and 1500 Daltons.

Referring to block 352 of FIG. 3D, in some embodiments, a first synthesis task in the plurality of synthesis tasks specifies an amount of a first reactant 1910-1 as a number of equivalents relative a second reactant 1910-2 specified by the first synthesis task to be added to the well specified by the first synthesis task. For instance, in some embodiments, the first synthesis task is known to yield a certain result when performed using the second reactant, but has not yet been performed using the first reactant, which allows for determining an amount of the first reactant to use when for the reaction based on the second reactant. Accordingly, in some embodiments, the method 300 determines the number of equivalents between the first reactant relative to the second reactant. As a non-limiting example, in some embodiments, the method 300 determines the number of equivalents between the first reactant and the second reactant based on a stoichiometric relationship between the first reactant and the second reactant. However, the present disclosure is not limited thereto. In some embodiments, the number of equivalents is associated with a number of reactants, in a plurality of reactants, which are synthetic equivalents of the respective synthon.

Referring to block 354, in some embodiments, a first synthesis task in the plurality of synthesis tasks specifies an amount of a first reactant 1910 as a number of moles of the first reactant 1910 to be added to the well 812 specified by the first synthesis task. For instance, referring briefly to FIG. 19, a user interface 1900 is presented through the computing system 100 that allows for an end-user to configure the first synthesis task for a well 812 associated with x-y address D6 of the multi-well plate 800, which allows the end-user to specify the amount of the first reactant 1910 added to the well 812 of 0.02 mol and an amount of 5 mmol of a second reactant. However, the present disclosure is not limited thereto.

Referring to block 356, in some embodiments, the first synthesis task in the plurality of synthesis tasks specifies an amount of a first reactant 1910-1 as a mass of the first reactant to be added to the well 812 specified by the first synthesis task. For instance, in some embodiments, the first synthesis task specifics the mass of the first reactant to be added to the well 812 and/or a process for adding the mass of the first reactant 1910-1 to the well 812, such as a first process for adding the first reactant 1910-1 as in bulk to a well 812, a second process titrating the mass of the first reactant into the well 812, or the like.

Referring to block 358, in some embodiments, the corresponding specification of the synthesis task further includes a reaction task. For instance, in some embodiments, in order to utilize and/or perform a reaction with a respective reactant, one or more reaction tasks must be completed during the workflow, such as a one or more dilution tasks, one or more filtration tasks, one or more plating tasks, or a combination thereof, in order for the reaction to complete successfully.

Referring to block 360, in some embodiments, the reaction task is an agitation task, a mixing task, a liquid chromatography task, or a temperature gradient task. In some embodiments, the reaction task is an agitation task, a mixing task, a liquid chromatography task, a temperature gradient task, or a combination thereof. For instance, in some embodiments, the agitation task mixes the solution 2920, such as by physically mixing the reactant 1910 and the solvent 1920 within the well 812. In some embodiments, the agitation task displaces the well 812, such as by applying one or more vibrations through a surface of the well 812, which causes a flow within the solution 2920 of the well 812. However, the present disclosure is not limited thereto. In some embodiments, the agitation task, the mixing task, the liquid chromatograph task, or the temperature task is performed using one or more instruments selected from the group consisting of: liquid handlers, shakers, heaters, robotic arms, decappers, plate sealers, barcode readers, and/or analyzers.

Referring to block 362, in some embodiments, the method 300 includes performing at least a respective subset of the plurality of synthesis tasks of the initial workflow. For instance, in some embodiments, the respective subset is performed at a molecular foundry that includes a plate handler for the first multi-well plate and one or more liquid handlers for at least the plurality of reactants, which allows for localizing where the initial workflow is performed to a single spatial region. In some embodiments, the performing the respective subset of the plurality of synthesis tasks includes reacting the plurality of reactants in the plurality of wells in accordance with the corresponding specification of at least the respective subset of the plurality of synthesis tasks. In some embodiments, the respective subset of the plurality of synthesis tasks includes each synthesis task associated with at least one well 812 of the multi-well plate 800, which allows for completing the reaction associated with the at least one well 812 through performing the respective subset of the plurality of synthesis tasks. However, the present disclosure is not limited thereto. In some embodiments, the respective subset of the plurality of synthesis tasks includes less than all each synthesis task associated with at least one well 812 of the multi-well plate 800, which represents intermittent reaction data.

Referring to block 363 of FIG. 3E, in some embodiments, the method 300 includes informing a first data set associated with a physical property of a liquid in each well 812 specified by the corresponding specification of each synthesis task in at least the respective subset set of synthesis tasks. For instance, in some embodiments, the corresponding specification associated with the molecular reaction 132 is informed by comparing and/or updating the stored data against the first data set obtained when performing the reaction, which allows for creating state-of-the-art data sets and conducting evaluations based on differences between the stored (e.g., historical data and/or theoretical data) against the observed data from the first data set. However, the present disclosure is not limited thereto.

Referring to block 364, in some embodiments, the physical property is determined using spectroscopy, which allows for quantitatively determining the physical property of the liquid based on an absorbance of one or more wavelengths of light when the multi-well plate 800 is illuminated, such as with a first visible light wavelength, a second infrared wavelength, or the like. Moreover, use of spectroscopy allows for conducting the method 300 using the computing system 100 and further without human intervention, which at least reduces at risk of contamination of the liquid and the like.

Referring to block 366, in some embodiments, the spectroscopy is ultraviolet (UV) spectroscopy and the physical property is absorbance of UV light. For instance, in some embodiments, the absorbance of UV light by one or more compounds in a respective well 812 allows for determining one or more analytes of various photochemical reactions in the respective well 812 or the determining absorbance of UV light longitudinally with time. However, the present disclosure is not limited thereto.

Referring to block 368, in some embodiments, the spectroscopy is light spectroscopy and the physical property is absorbance of visible light (e.g., a wavelength between about 380 nm and about 750 nm). For instance, in some embodiments, the absorbance of visible light is associated with a first band of wavelengths in the visible light spectrum (e.g., a first red band between 620 nm and 750 nm, a first blue band between 450 nm and 495 nm, etc.), which allows for obtaining observable information associated with absorption of discrete bands of light.

Referring to block 370, in some embodiments, the spectroscopy is infrared (IR) spectroscopy and the physical property is absorbance of IR light. For instance, in some embodiments, the absorbance of IR light allows for the physical property to define a molecular structure of a target compound, one or more functional groups of the target compound, evaluating reaction monitoring, and/or the like.

Referring to block 372, in some embodiments, the spectroscopy is atomic absorption spectroscopy and the physical property is absorbance of light.

Referring to block 374, in some embodiments, the spectroscopy is inductively coupled plasma optical emission spectroscopy (ICP-OES) and the physical property is light emission. For instance, in some embodiments, ICP-OES is utilized to evaluate one or more trace materials associated with a respective reactant 1910, solvent 1920, solution 2920, and/or target compound.

Referring to block 376, in some embodiments, the spectroscopy is fluorescence spectroscopy and the physical property is light emission, which allows for fluorescent microscopy images of the solution 2920 within a respective well 812 to be captured, such as by a sensor of the computing system 100 (e.g., a two-dimensional pixelated sensor) or a vision module of the computing system 100. As a non-limiting example, in some embodiments, a

Referring to block 378, in some embodiments, the spectroscopy is Raman spectroscopy and the physical property is vibrational or rotational model of atoms of the target compound.

Referring to block 380 of FIG. 3F, in some embodiments, the performing (e.g., 362 of FIG. 3D) the respective subset of the plurality of synthesis tasks and/or the informing (e.g., block 363 of FIG. 3E) the first data set associated with the physical property is conducted without human intervention. For instance, in some embodiments, the performing the respective subset of the plurality of synthesis tasks and the informing the first data set associated with the physical property of the method 300 are performed recursively or for a predetermined number of instances, such as until a threshold number of evaluations are performed on a respective well 812, an epoch has elapsed, a threshold temperature is achieved, a threshold pipettor submerge depth is achieved, or the like.

Referring to block 382, in some embodiments, the first data set includes a first plurality of data elements associated with the field of view prior to the illuminating and a second plurality of data elements associated with the field of view when illuminated during the informing the first data set associated with the physical property (e.g., block 363 of FIG. 3E). Accordingly, the first data set allows for performing a statistical analysis on the solution 2920 accommodated by the well 812 within the field of view based on differences when the well 812 is and/or is not illuminated, such as a fluorescent imaging evaluation, an infrared imaging evaluation, a UV imaging evaluation, or the like. Additionally, this allows for capturing graphical data associated with the well 812, which provides complex, valuable information when applied to one or more models 160, such as applied as an input data set to a first model 160-1 configured to evaluate a conversion value of a reaction. However, the present disclosure is not limited thereto.

Referring to block 384, in some embodiments, the first data set includes a first plurality of data elements associated with one or more reaction conversion rates when producing at least one target compounds in the plurality of target compounds. For instance, in some embodiments, a model 160 the computing system 100 determines a conversion rate when producing a first target compound in the plurality of target compounds and stores the conversion rate in the corresponding specification associated with the first target compound and/or the workflow. However, the present disclosure is not limited thereto. In some embodiments, when the conversion rate meets or exceeds the threshold conversion value (e.g., conversion value 154 of FIG. 2B), the respective instance of the molecular reaction is selected for retention in the subset of instances and the corresponding specification is informed. In some embodiments, when conversion rate does not meet the threshold conversion value, the respective instance of the molecular reaction is not selected for the subset of instances and the corresponding specification is not information. However, the present disclosure is not limited thereto.

In some embodiments, the conversion rate is determined as a percent yield of a corresponding target compound obtained by the initial workflow. In some embodiments, the conversion rate is a percent yield of a corresponding target compound obtained for the respective instance of the molecular reaction determined as a ratio of product to starting material. In some embodiments, the conversion rate is determined as a percent of a remaining amount (e.g., mass, volume) of one or more solvent 1920 and/or one or more reactants 1910 (e.g., a first reactant and/or a second reactant) obtained for the respective instance of the molecular reaction. For instance, in an example embodiment, where 80% of a first solvent 1920 and/or a first reactant 1910 is consumed in the respective instance of the molecular reaction, the percent of a remaining amount of the first solvent 1920-1 and/or the first reactant 1910-1 is 20%. In some embodiments, the conversion rate is determined as a percent consumption (e.g., mass, volume) of one or more solvents 1920 and/or one or more reactants 1910 (e.g., a first reactant and/or a second reactant) obtained for the respective instance of the molecular reaction. For instance, in an example embodiment, where 80% of a first solvent 1920-1 and/or a first reactant 1910-1 is consumed in the respective instance of the molecular reaction, the percent consumption of the first solvent 1920 and/or the first reactant 1910 is 80%.

Referring to block 386, in some embodiments, the first data set includes at least one corresponding alphanumeric identifier for each target compound in the plurality of target compounds that identifies a well 812 in the multi-well plate 800 that the initial workflow specifies contains the target compound (e.g., identifier 1930 of FIG. 23). For instance, in some embodiments, the alphanumeric identifier corresponds to the x-y address 1930 of the well 812 in the multi-well plate 800. As a non-limiting example, referring to FIG. 19, in some embodiments, a first well 812-1 is associated with a corresponding alphanumeric identifier D6, which is determined based on a horizontal position 808 and/or vertical position 806 of the first well 812-1 relative to an origin or initial position of the x-y position system.

Referring to block 388-390, in some embodiments, the first data set includes at least one corresponding set of Cartesian coordinates for each target compound in the plurality of target compounds that identifies the well 812 in the multi-well plate 800 that the initial workflow specifies contains the target compound. For instance, in some embodiments, the corresponding set of Cartesian coordinates define a first horizonal position (e.g., an X position), a second horizontal position (e.g., a Y position), a third vertical position (e.g., a Z position), or a combination thereof of the well 812 on a surface of the multi-well plate 800. For instance, in some embodiments, an origin of the Cartesian coordinates is associated with an indicia or marker on the surface of the multi-well plate 800, which provides positional tolerance, such as when a pipettor 2910 traverses between a first well 812-1 and a second well 812-2 of the multi-well plate 800. However, the present disclosure is not limited thereto.

In some embodiments, the first data set includes one or more spatial coordinates associated for each target compound in the plurality of target compounds that identifies the well 812 in the multi-well plate 800 that the initial workflow specifies contains the target compound, a temporal identifier of an amount of time in the corresponding specification of a synthesis task associated with the respective target compound, a spectral identifier of one or more wavelengths associated with the respective target compound, or a combination thereof.

Referring to block 392 of FIG. 3G, in some embodiments, at least two synthesis tasks in the plurality of synthesis tasks are required to synthesize a first target compound in the plurality of target compounds and the performing (e.g., 362 of FIG. 3D) the respective subset of the plurality of synthesis tasks and/or the informing (e.g., block 363 of FIG. 3E) the first data set associated with the physical property is performed a first time for a first synthesis task in the at least synthesis two tasks and the performing the respective subset of the plurality of synthesis tasks and informing the first data set associated with the physical property is performed a second time, after the first synthesis task, for a second synthesis task in the at least two synthesis tasks.

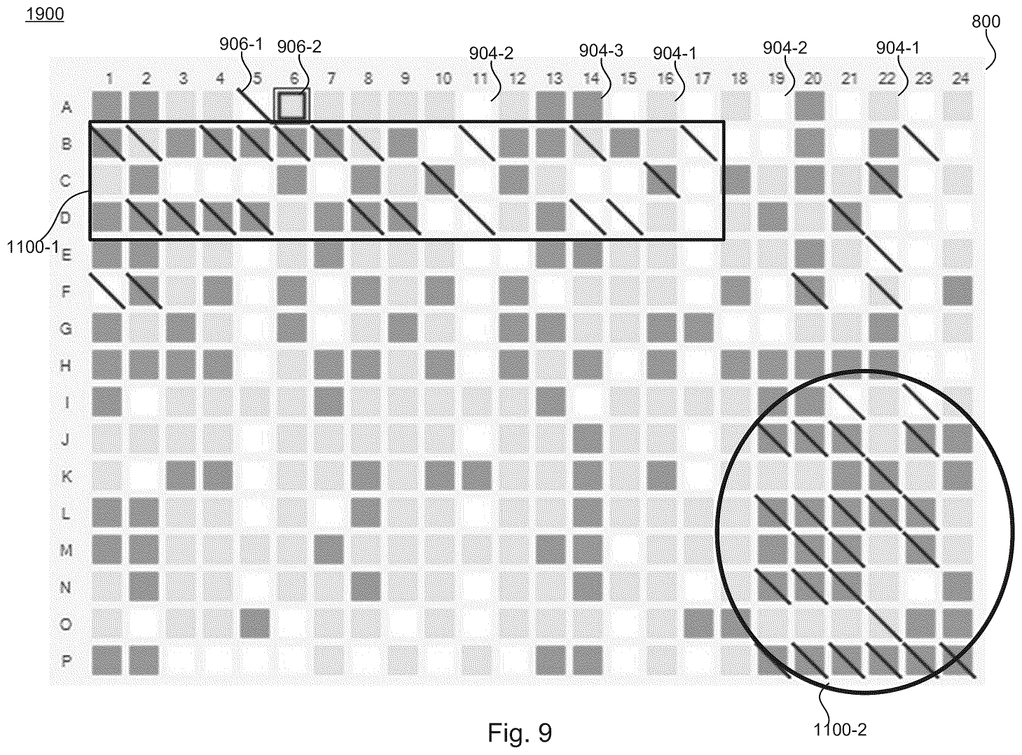

Referring to block 394, in some embodiments, the method includes repeating the performing (e.g., block 362 of FIG. 3D) the respective subset of the plurality of synthesis tasks and obtaining (e.g., block 302 of FIG. 3A) until each synthesis task in the plurality of synthesis tasks has been performed. For instance, in some embodiments, some reactions are multistep reactions (e.g., at least 10 steps performed in a series order). For instance, in some embodiments, a first step of a first reaction requires reacting a first reactant with a second reactant 2 for reaction time A in a first well (e.g., well P1 of FIG. 8, well A1 of FIG. 9, etc.) to yield intermediate B, a second step of measuring a physical property of intermediate B in the first well (e.g., with UV light illuminated in a field of view including the first well), a third step of reacting the intermediate B with a third reactant C in the first well for a second reaction time B to form the target molecule, and a fourth step of measuring a physical property of the target molecule in the first well to quantify an efficiency of the overall reaction. In some embodiments, the first reaction is encoded by the first synthesis task, the second reaction is encoded by the second synthesis task, and the like.

Referring to block 396, in some embodiments, the method 300 includes determining an amount of each target compound in the plurality of target compounds that was synthesized in accordance with the plurality of synthesis tasks using the first data set. For instance, in some embodiments, the method 300 determines a mass of each target compound using a mass sensor, such as a scale associated with the computing system 100, that was produced when performing the workflow 2400.

Referring to blocks 398-402 of FIG. 3H, in some embodiments, the method 300 includes using the amount of each target compound in the plurality of target compounds that was synthesized to amend the corresponding specification of each synthesis task in the plurality of synthesis tasks. For instance, in some embodiments, a predetermined amount of each target compound is determined prior to the performing (e.g., 362 of FIG. 3D) the respective subset of the plurality of synthesis tasks and/or the informing the first data set associated with the physical property (e.g., block 363 of FIG. 3E) and the method 300 evaluations the amount of each target compound that was synthesized in order to determine if a future instance or repeating of the performing the respective subset of the plurality of synthesis tasks or the like requires improvement and/or reiteration. However, the present disclosure is not limited thereto.

In some embodiments, the using the amount of each target compound in the plurality of target compounds that was synthesized to amend the corresponding specification includes training the model 160 using the corresponding specification. In some embodiments, the model 160 is configured to estimate a target compound synthetic efficiency as a function of the synthesis task specification. In some embodiments, the model 160 includes a plurality of parameters (e.g., parameters 162 of FIG. 2B), which collectively, through application of a procedure, provides for applying the synthesis task specification of one or more synthesis tasks in the plurality of synthesis tasks for the synthesis of a corresponding target compound in the plurality of target compounds in order to obtain a calculated synthetic efficiency for the corresponding target compound. In some embodiments, the model 160 determines a difference between (a) an efficiency of the corresponding target compound as determined by the model 160 and (b) an actual efficiency of the corresponding target compound as determined by the first data set. In some embodiments, the model 160 back-propagating the difference through the plurality of the models 160 of the computer system 100, which trains the model 160. In some embodiments, the procedure is repeated for each target compound, or batch of target compounds, in the plurality of target compounds.

Referring to block 404, in some embodiments, the plurality of synthesis tasks encodes a plurality of different specifications for synthesizing a first target compound in the plurality of target compounds. Moreover, in some embodiments, the using (e.g., block 398 of FIG. 3H) includes pruning out a first subset of the plurality of different specifications for synthesizing the first target compound from the initial synthesis tasks that the first data set indicates synthesized the first target compound at a lower efficiency than a second subset of the plurality of different specifications for synthesizing the first target compound. For instance, in some embodiments, the model 160 prunes out the first subset of the plurality of different specifications based on a vector analysis (e.g., cosine similarity) between the first target compound and one or more specifications in the second subset of the plurality of different specifications. However, the present disclosure is not limited thereto.

### Example Methods for Visualizing Reaction Conversion

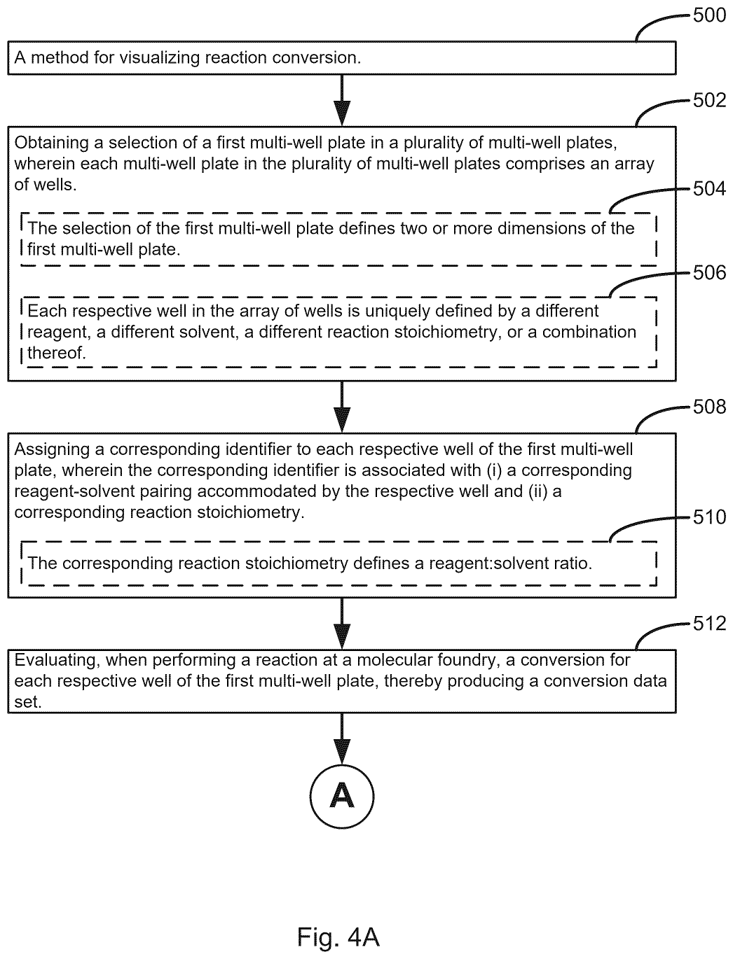

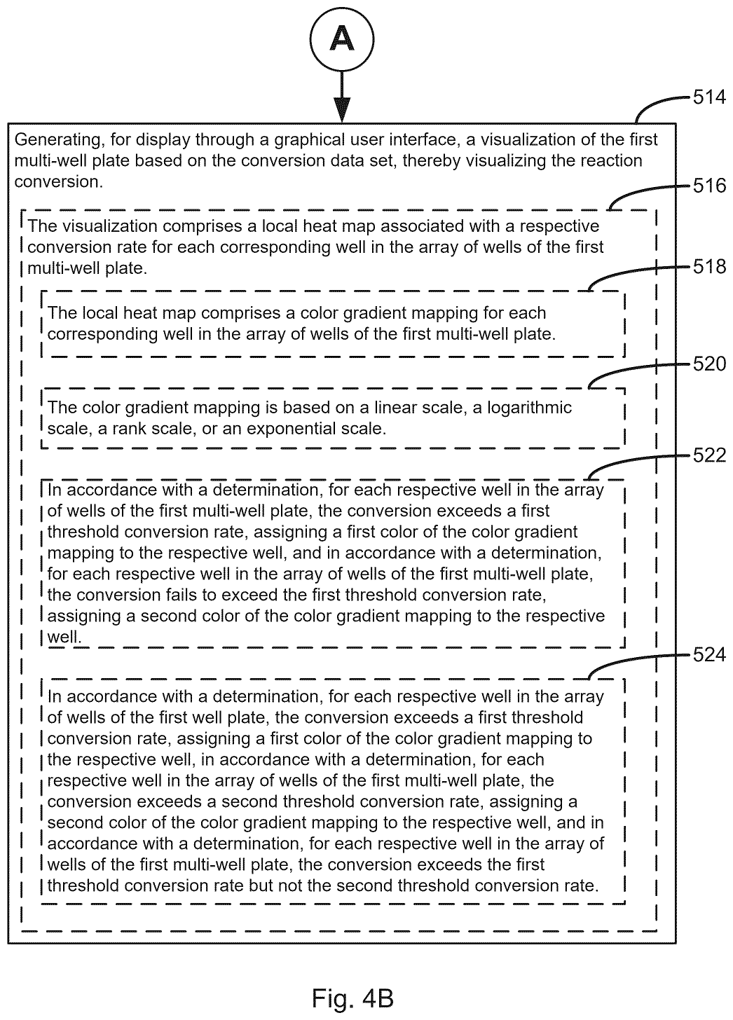

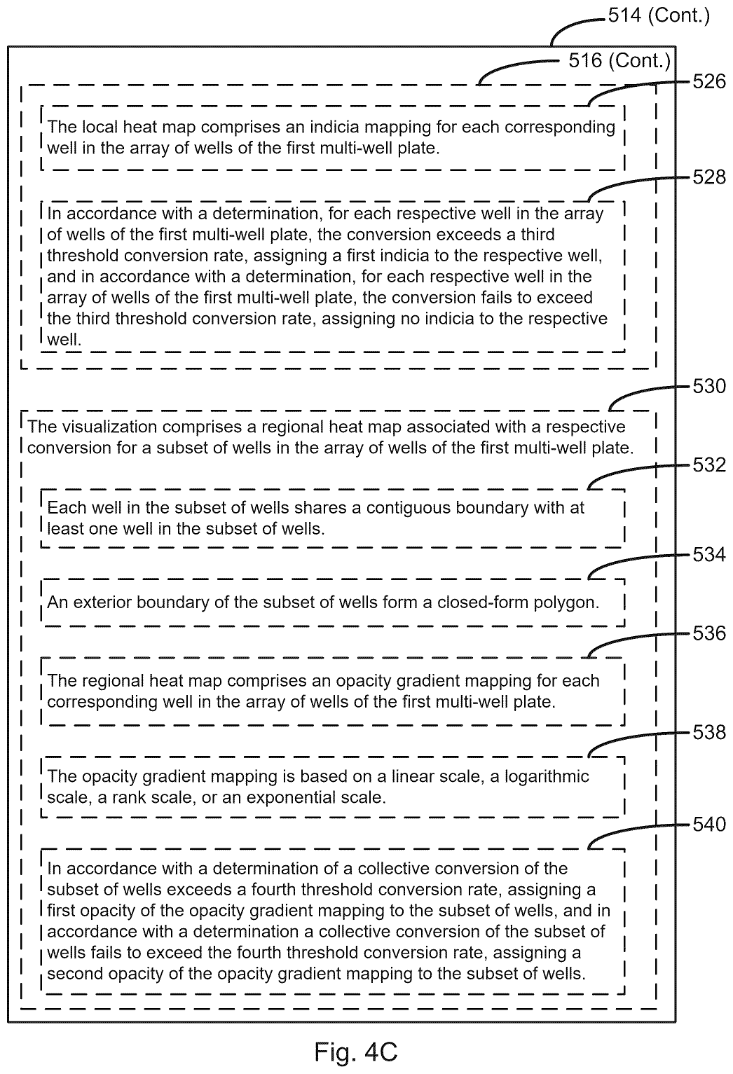

As illustrated in FIGS. 4A-4C, another aspect of the present disclosure provides a method for visualizing reaction conversion, such as synthesis of a compound using a molecular reaction. In some embodiments, the molecular reaction is a multistep molecular reaction.

Referring to block 500 of FIG. 4A, in some embodiments, the present disclosure provides a method 500 for visualizing reaction conversion (e.g., block 384 of method 300 of FIG. 3).

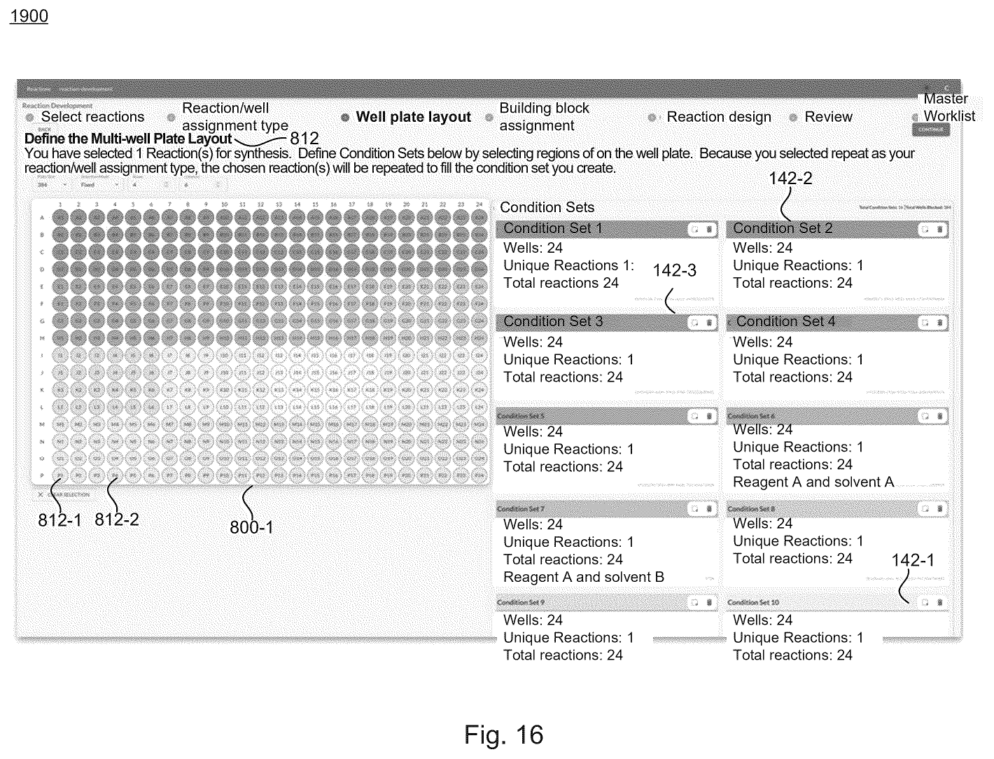

Referring to block 502, in some embodiments, the method 500 includes obtaining a selection of a first multi-well plate (e.g., multi-well plate 800 of FIG. 8, multi-well plate 800 of FIG. 9, multi-well plate of FIG. 16, etc.) in a plurality of multi-well plates 800. In some embodiments, each multi-well plate 800 in the plurality of multi-well plates 800 includes an array of wells 812, each of which is configured to accommodate a corresponding liquid solution (e.g., solution 2920 of FIG. 29, etc.). For instance, in some embodiments, the array of wells includes between 50 and 400 wells 812 (e.g., block 312 of FIG. 3A), which allows for performing (e.g., block 362 of FIG. 3D) various synthesis tasks at a molecular foundry using a single multi-well plate. For instance, referring briefly to FIG. 17, an end-user of a user interface 1900 provides an option to select a respective number of wells 812 in the array of wells 812 of the multi-well plate 800, in which the option for 384 is selected and 130 of the wells 812 are configured for performing a reaction within a corresponding well 812.

Referring to block 504, in some embodiments, the selection of the first multi-well plate 800-1 defines two or more dimensions of the first multi-well plate 812-1, such as a horizontal position (e.g., column 808 of FIG. 8) and/or a vertical position (e.g., row 806 of FIG. 8) within a matrix. For instance, in some embodiments, the end-user of the computing system 100 defines a first number of rows 806 and/or a second number of columns 808 associated with the multi-well plate 800, which defines a number of wells 812 in the array of wells 812. However, the present disclosure is not limited thereto. For instance, in some embodiments, the end-user selections from a listing of predetermined sizes of multi-well plates, such as a first size associated with a first multi-well plate 800-1 having 96 wells 812, a second size associated with a second multi-well plate 800-2 having 384 wells 812, and the like. Moreover, in some embodiments, the end-user of the computing system 100 defines a first number of rows 806 and/or a second number of columns 808 for performing a reaction from the available wells 812 of the multi-well plate 800, which defines a number of wells 812 in the array of wells 812. Accordingly, in some such embodiments, the method 500 allows for the end-user to select from one or more template multi-well plate 800 sizes for ease of configuration by the end-user when configuration coarse parameters and/or fine parameters associated with the reactions performed at the multi-well plate 800.

Referring to block 506, in some embodiments, each respective well 812 in the array of wells 812 is uniquely defined by a different reagent (e.g., first reactant 1910 of FIG. 19), a different solvent (e.g., first solvent 1920 of FIG. 19), a different reaction stoichiometry, or a combination thereof. For instance, in some embodiments, no two wells 812 of the multi-well plate performs the same reaction during when executing a first workflow (e.g., workflow 2400 of FIG. 3A, workflow 2400 of FIG. 24, etc.). However, the present disclosure is not limited thereto.

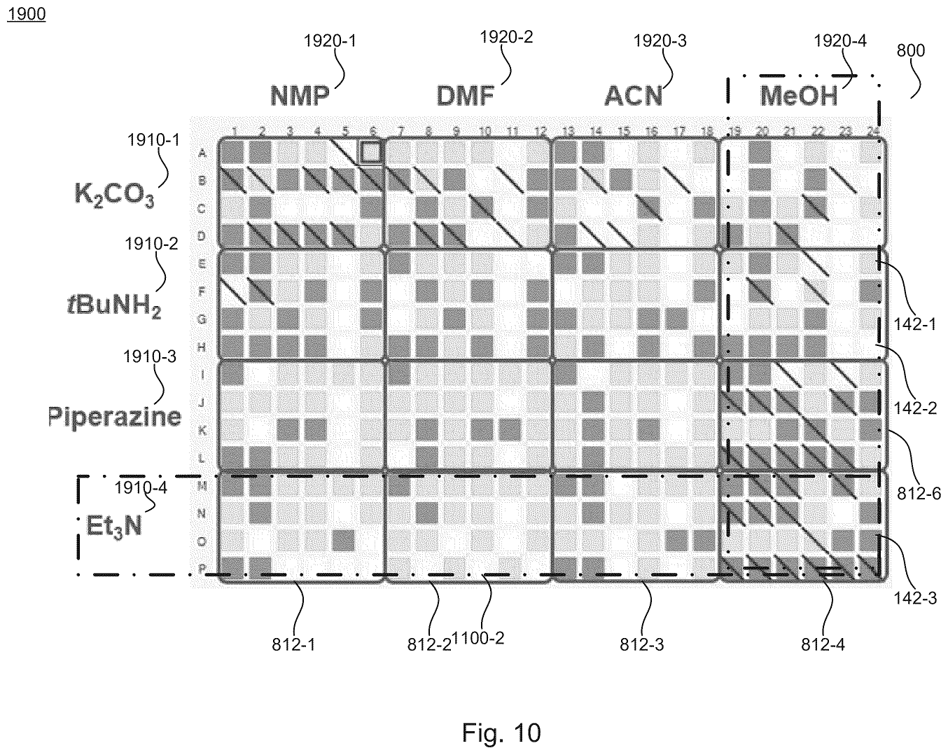

Referring to block 508, in some embodiments, the method 500 includes assigning a corresponding identifier (e.g., address and/or identifier 1930 of FIG. 19) to each respective well 812 of the first multi-well plate 812-1. In some embodiments, the corresponding identifier is associated with (i) a corresponding reagent-solvent pairing accommodated by the respective well and (ii) a corresponding reaction stoichiometry. For instance, referring briefly to FIGS. 10-12, each x-axis address 808 is associated with a corresponding solvent 1920 and each y-axis address 806 is associated with a corresponding reactant 1910, which creates pairings to product the target compound(s). As a non-limiting example, a sixth well 812-6 of FIG. 10 assigned a corresponding identifier based on the corresponding reagent-solvent pairing of methanol (MeOH) and piperazine (e.g., an intersection of a first subset of wells 1100-1 associated with a fourth solvent 1920-4 identified through a dot-dot-dash polygon indicia of the user interface 1900 and second subset of well 1100-2 associated with a fourth reagent 1910-4 identified through a dot-dash polygon indicia of the user interface 1900). Moreover, in some embodiments, since the sixth well 812-6 is at an edge portion of the multi-well plate and/or a boundary of a respective subset of well 812, the sixth well 812-2 represents an extrema of the spectrum of reaction stoichiometry for either a respective row 806 and/or column 808, which aids with informing evaluations of conversion value efficiency or the like.

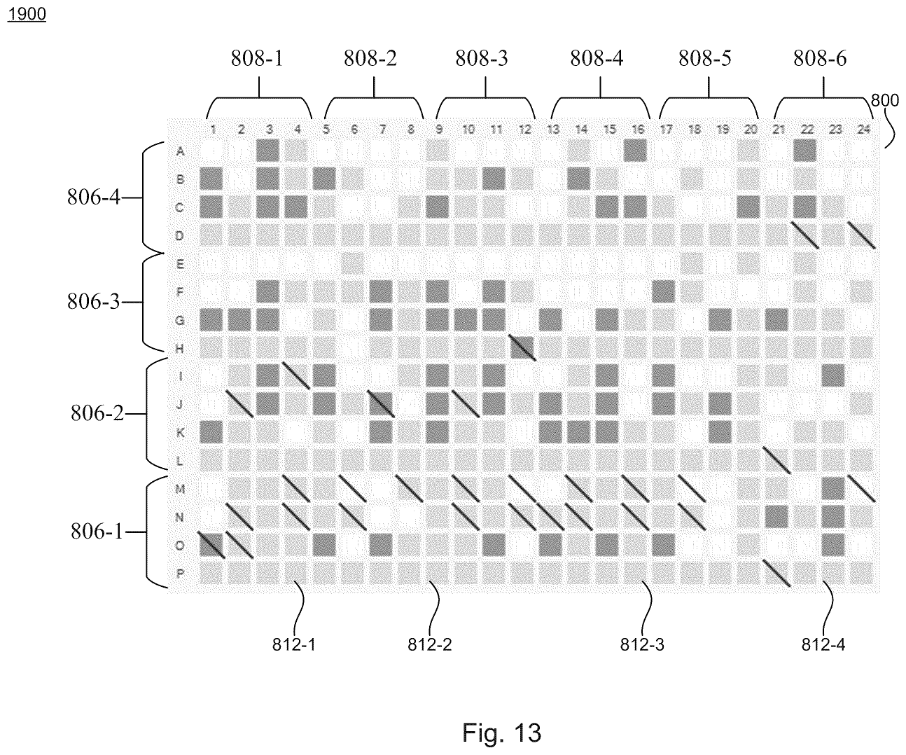

Referring to block 510, in some embodiments, the corresponding reaction stoichiometry defines a reagent-solvent ratio. For instance, in some embodiments, the corresponding reagent-solvent pairing is associated with a subset of wells 1100 of the multi-well plate 800, such as a sub-array in the array of wells 812 or a subset of rows 806 and/or columns 808. By way of example, in FIG. 11, each of a first well 812-1 and a third well 812-3 are associated with a subset of wells 1100 corresponding to a triethylamine (Et₃N):dimethylformamide (DMF) pairing, in which the first well 812-1 is associated with a first ratio of Et₃N:DMF (e.g., 3:1) and the third well 812-3 is associated with a second ratio (e.g., 3:2). However, the present disclosure is not limited thereto. As another non-limiting example, referring to FIG. 13, each well 812 associated with a first subset of rows 806-1 is associated with a first reagent stoichiometry (e.g., 1:1), each well 812 associated with a second subset of rows 806-2 is associated with a second reagent stoichiometry (e.g., 1:2), and each well 812 associated with a fourth subset of rows 806-4 is associated with a fourth reagent stoichiometry (e.g., 2:8). Moreover, each well 812 associated with a first subset of columns 808-1 is associated with a first reagent stoichiometry (e.g., 1:1), each well 812 associated with a second subset of columns 808-2 is associated with a second reagent stoichiometry (e.g., 1:1.5), and each well 812 associated with a sixth subset of columns 808-6 is associated with a fourth reagent stoichiometry (e.g., 1:4). However, the present disclosure is not limited thereto. Accordingly, the method 500 allows for performing reactions with the same reagent-solvent pairing at a variety of reagent-solvent ratios in order to determine an optimal ratio for a particular workflow.

Referring to block 512, in some embodiments, the method 500 includes evaluating, when performing a reaction at a molecular foundry (e.g., block 362 of FIG. 3D), a conversion for each respective well 812 of the first multi-well plate 800-1. For instance, in some embodiments, the evaluating of the conversion occurs during (e.g., concurrent and/or simultaneous to the performing the respective subset of the plurality of synthesis tasks), which allows for producing a conversion data set immediately after or when the reaction occurs, aiding in the evaluation of the conversion rate. In some embodiments, the evaluation is performed by one or more models (e.g., model construct 160 of FIG. 2A), which allows for tailoring the evaluation based on a prior training of the model 160, such as a training of one or more classifications associated with a reactant 1910, solvent 1920, and/or solution 2920.

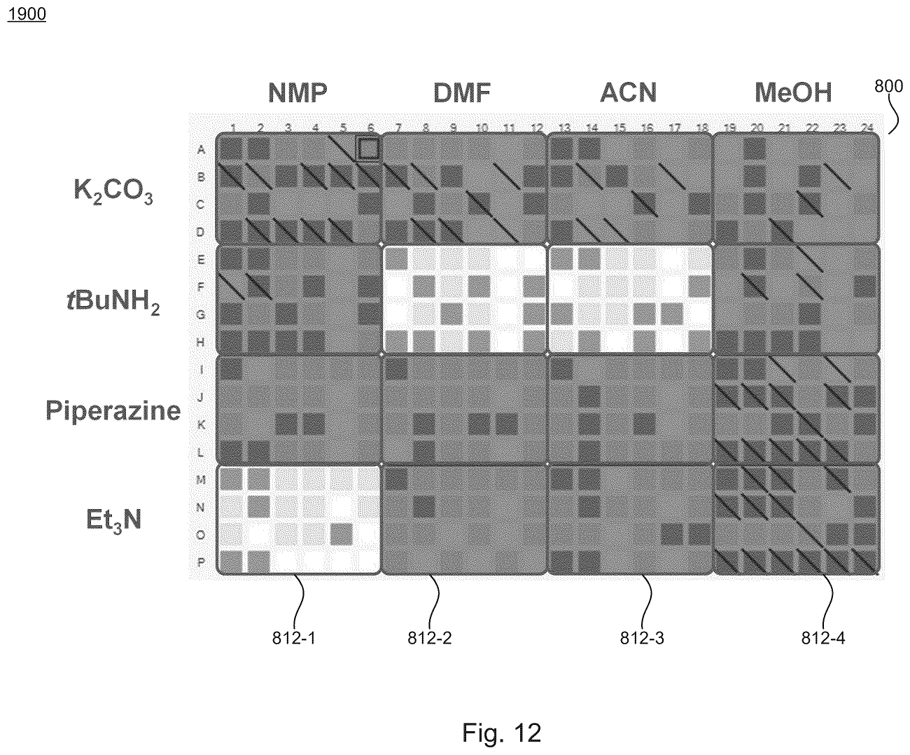

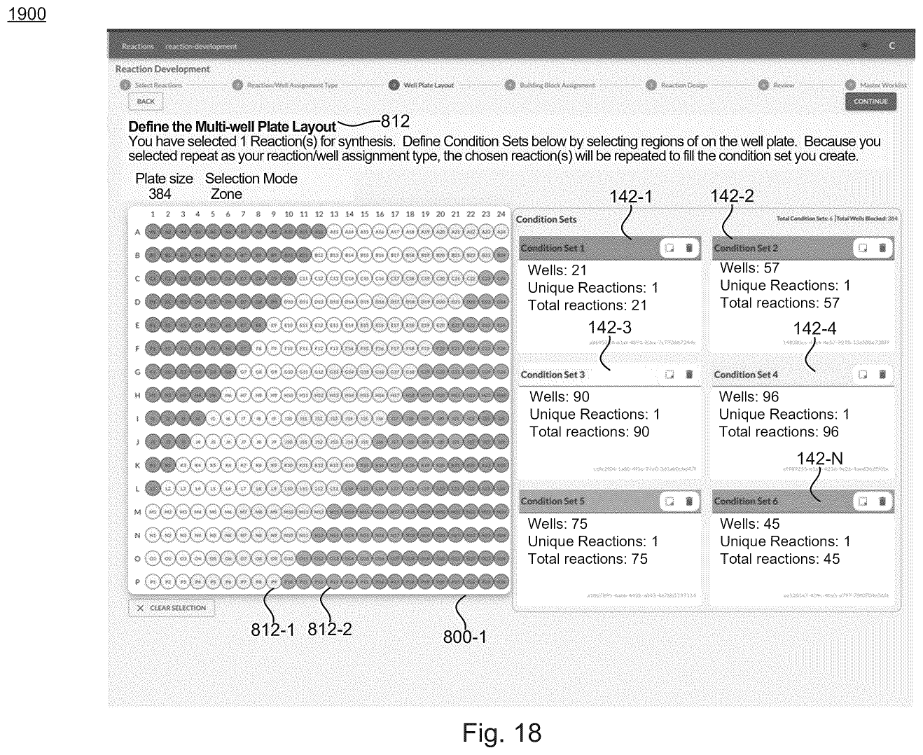

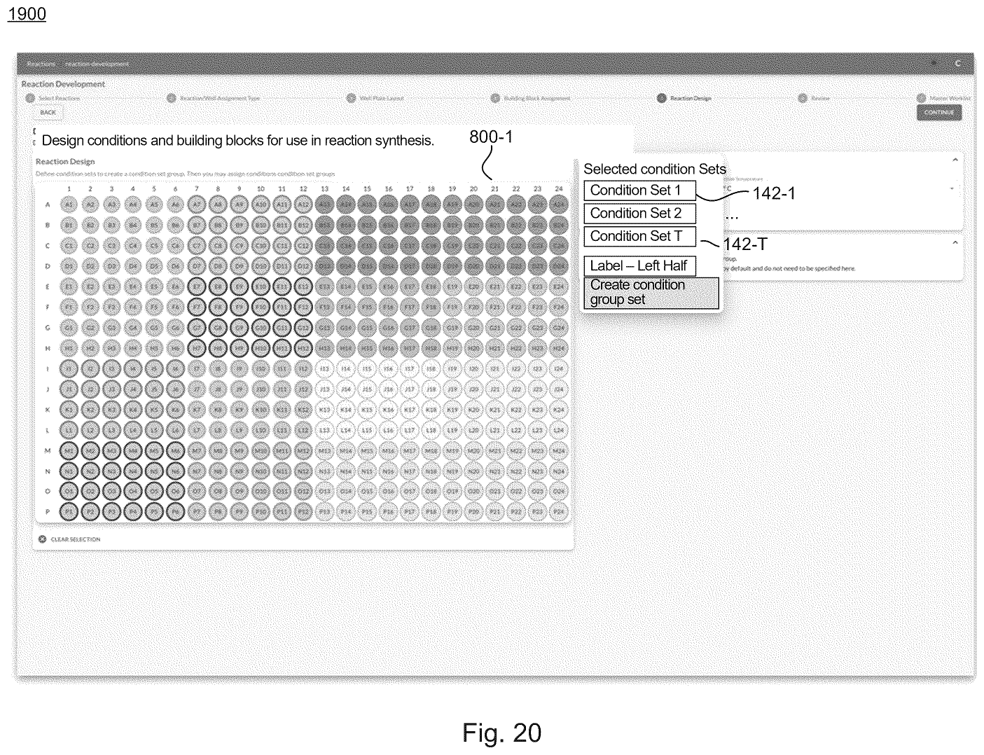

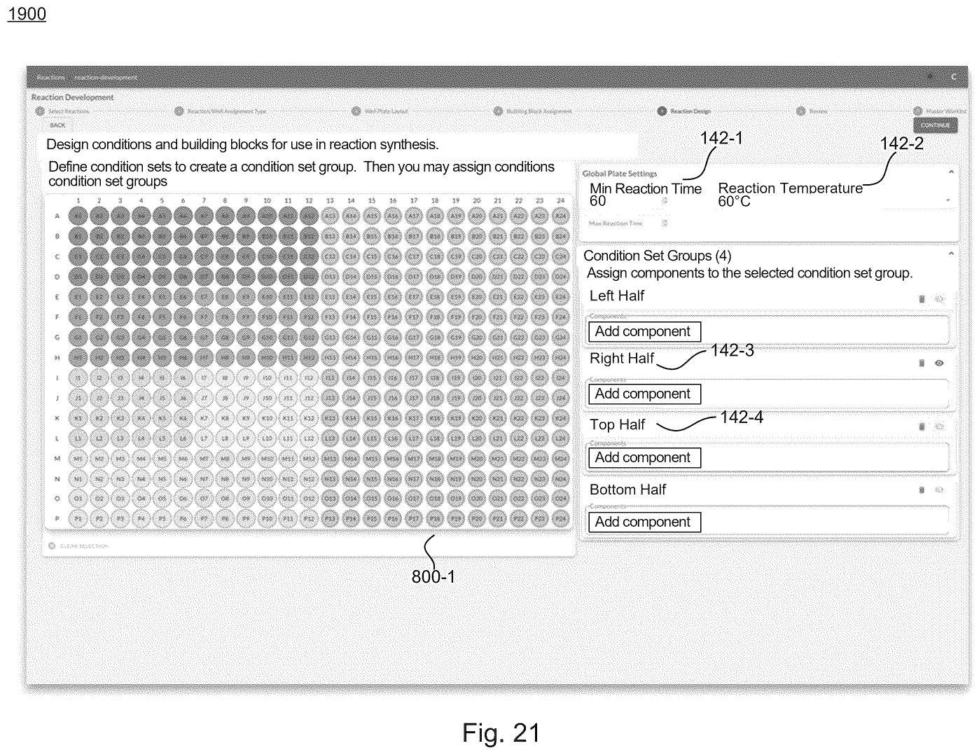

Referring to block 514 of FIG. 4B, in some embodiments, the method 500 includes generating a visualization of the first multi-well plate 800-1 based on the conversion data set, which allows for an end-user to visualize the reaction conversion (e.g., user interface 1900 of FIG. 8, user interface 1900 of FIG. 9, user interface 1900 of FIG. 11, user interface 1900 of FIG. 12, user interface 1900 of FIG. 13, user interface 1900 of FIG. 16, user interface 1900 of FIG. 17, user interface 1900 of FIG. 18, user interface 1900 of FIG. 19, user interface 1900 of FIG. 20, user interface 1900 of FIG. 21, user interface 1900 of FIG. 22, user interface 1900 of FIG. 23, etc.). For instance, referring to FIGS. 8-13, in some embodiments, the visualization comprises a two-dimensional view of the multi-well plate 800, such as a top view or plane view of the multi-well plate 800, which allows the end-user to graphically observer each well 812 of the multi-well plate 800 through the visualization without emphasizing any one well 812 of another 812.

Referring to block 516, in some embodiments, the visualization includes a local heat map associated with a respective conversion rate for each corresponding well 812 in the array of wells of the first multi-well plate 800-1. For instance, in some embodiments, a region associated with each well 812 of the multi-well plate 800 is assigned a corresponding color from a plurality of colors, in which at least two colors in the plurality of colors is assigned to different wells 812, which allows for visual gradients to be observed from the heat map.

Referring to block 518, in some embodiments, the local heat map includes an indicia gradient mapping, such allows for visualizing differentials and changes in information's based for a particular area, such as for the first multi-well plate 800-1 for a subset of wells 1100 of the multi-well plate. For instance, in some embodiments, an indicia gradient mapping provides a color gradient mapping that shows variations in information associated with the wells 812 using a plurality of colors to indicate different values of information for each corresponding well in the array of wells 812 of the first multi-well plate 800-1. For instance, in some embodiments, the color gradient mapping includes a plurality of colors, in which each respective well 812 of the first multi-well plate 800-1 is assigned a corresponding color based on a determination of a reaction failure, success, and/or intermediate result. In some embodiments, the color gradient mapping includes a shading, a hue, a tint, a color, or a combination thereof. For instance, referring FIG. 11, a first red color is associated with each failed reaction in a corresponding well 812, a second yellow color is associated with each intermediate result reaction in the corresponding well 812, and a third green color for successful reactions in the corresponding well 812. However, the present disclosure is not limited thereto. Example indications of conversion in accordance with some embodiments of the present disclosure (e.g., black for fail or 0% conversion, gray for success or greater than 50% conversion, light gray for intermediate success or greater than 0% and less than or equal to 50% conversion). Other methods of indicating conversion are possible, as will be apparent to one skilled in the art.

By way of example, in some embodiments, the indicia gradient mapping uses one or more indicia, one or more shades of a color, one or more hues of a color, one or more polygonal shapes, or a combination thereof to form the visualization including the heat map. For instance, in some embodiments, the first data set is utilized obtain a respective value for each well 812 of the multi-well plate 800. In some embodiments, the model 160 assigns a corresponding indicia, a corresponding shade of a color, a corresponding hue of a color, a corresponding polygonal shape, or a combination thereof to each respective well 812. Accordingly, the visualization forms the heat map with the gradient based on the visual transitions and/or patterns formed between the wells 812 and the one or more indicia, the one or more shades of the color, the one or more hues of the color, the one or more polygonal shapes, or the combination thereof. For instance, referring to FIG. 9, in some embodiments, a first indicia 906-1, which appears in the graphical user interface 1900 as a slash, or diagonal, line through the icon associated with a corresponding well 812 is utilized to indicate the corresponding well 812 failed to deem complete the corresponding target compound reaction. In some embodiments, a second indicia 906-2, which appears in the graphical user interface as rectangular polygon surrounding an edge portion of the icon associated with a corresponding well 812 is utilized to indicate the corresponding well 812 satisfied the corresponding target compound reaction. However, the present disclosure is not limited thereto. For instance, in some embodiments, the indicia gradient mapping uses one or more graphical icons, such as one or more shapes and/or emoticons that is applied to a corresponding region or icon associated with a respective well 812. Moreover, in some embodiments, the first color is associated with each reaction in a corresponding well 812 that yielded 0% conversion, a second color is associated with each intermediate result reaction in the corresponding well 812 that yielded less than 50% conversion but greater than 0%, and a third color for successful reactions in the corresponding well 812 that yielded greater than 50%. Accordingly, the indicia gradient mapping allows for using different types of indicia to visualize different types of information, such as graphical icon indicia for a first type of information (e.g., completion of a reaction) and color indicia for a second type of information (e.g., conversion of completed reactions) to allow and end-user to evaluate multiple parameters and data types by visualizing the heat map. However, the present disclosure is not limited thereto.

In some embodiments, each data point or position of the indicia gradient mapping has a one-to-one relationship with a well 812 of the first multi-well plate 800.

Referring to block 520, in some embodiments, the color gradient mapping is based on a linear scale, a logarithmic scale, a rank scale, or an exponential scale. For instance, in some embodiments, a model 160 utilizes one or more functions to assign a color to each respective well 812, such as a linear function that assigns a respective color from a plurality of colors based on a linear association between a first data set associated with a physical property of the solution 2920 in the respective well 812 and the plurality of colors, which allows for observing constant rate changes associated with the physical property in a graphical nature. In some embodiments, the model 160 utilizes a rank function that assigns a respective color from a plurality of colors based on a rank order of a first data set associated with a physical property of the solution 2920 in the respective well 812 and the plurality of colors, which allows for observing ranked changes associated with the physical property in a graphical nature. In some embodiments, the model 160 utilizes a logarithmic function that assigns the respective color from the plurality of colors based on a logarithmic association between the first data set associated with the physical property of the solution 2920 in the respective well 812 and the plurality of colors, which allows for observing changes at lower and upper end portions of the first data set associated with the physical property in a graphical nature. In some embodiments, the model 160 utilizes an exponential function that assigns the respective color from the plurality of colors based on an exponential association between the first data set associated with the physical property of the solution 2920 in the respective well 812 and the plurality of colors, which allows for observing changes at lower and upper end portions of the first data set associated with the physical property in a graphical nature.

Referring to block 522, in some embodiments, the method 500 includes assigning a first color of the color gradient mapping to the respective well 812 in accordance with a determination that the conversion exceeds a first threshold conversion rate. In some embodiments, the method 500 includes assigning a second color of the color gradient mapping to the respective well in accordance with a determination the conversion fails to exceed the first threshold conversion rate. In some embodiments, the determination is performed for each respective well 8112 in the array of wells of the first multi-well plate 812-1. For instance, in some embodiments, a first threshold conversion rate is associated with a conversion efficiency of 50% or and applies a first color if satisfied, a second threshold conversion rate is associated with a conversion efficiency between 1% and 49% and applies a second color if satisfied, and a third threshold conversion rate is associated with a conversion efficiency of 0% and applies a third color if satisfied. As a non-limiting example, referring briefly to FIG. 10, a first condition 142-1 of the multi-well plate 800 assigns a green color if the first threshold conversion rate satisfied, a second condition 142-2 assigns a yellow color if the second threshold conversion rate is satisfied but not the first threshold conversion rate, and a third condition 142-3 assigns a red color if the third threshold conversion rate is satisfied but not the first and second threshold conversion rates. However, the present disclosure is not limited thereto.

Referring to block 524, in some embodiments, the method 500 includes, in accordance with a determination, for each respective well 812 in the array of wells of the first multi-well plate 800-1, the conversion exceeds a first threshold conversion rate, assigning a first color of the color gradient mapping to the respective well, in accordance with a determination, for each respective well in the array of wells of the first multi-well plate, the conversion exceeds a second threshold conversion rate, assigning a second color of the color gradient mapping to the respective well, and in accordance with a determination, for each respective well in the array of wells of the first multi-well plate, the conversion exceeds the first threshold conversion rate but not the second threshold conversion rate.

Referring to blocks 526-528 of FIG. 4C, in some embodiments, the local heat map includes an indicia mapping for each corresponding well in the array of wells of the first multi-well plate. In some embodiments, in accordance with a determination, for each respective well 812 in the array of wells of the first multi-well plate 800-1, the conversion exceeds a third threshold conversion rate, assigning a first indicia 906 to the respective well 812, and in accordance with a determination, for each respective well 812 in the array of wells of the first multi-well plate 800-1, the conversion fails to exceed the third threshold conversion rate, assigning no indicia to the respective well 812. For instance, referring to FIG. 9, in some embodiments, a first indicia 906-1, which appears in the graphical user interface as a slash, or diagonal, line through the icon associated with a corresponding well 812 is utilized to indicate each well 812 of the multi-well plate 800 that satisfied a first threshold condition or, similarly, failed to satisfy a second threshold condition, such as a threshold conversion rate. Accordingly, by applying the first indicia to each well 812 that satisfies the threshold conversion rate, the visualization of the multi-well plate 800 allows an end-user to readily observe the reagent-solvent pairings and reagent-solvent ratios that produced optimal amounts of the target compound, as well as those that failed to do. For instance, in some embodiments, the end-user can evaluate observable patterns of the first indica applied to subsets of wells 1100. By way of example, referring to FIG. 9, a first subset of wells 1100-1 is associated with a first cluster of wells 812 and a second subset of wells 1100-2 is associated with a second cluster of wells 812, in which each 812 in the first and second clusters of wells 812 satisfies the threshold condition (e.g., has a conversion rate of less than 50%), which allows the end-user to visualize that the success and failure of certain reagents, solvents, and stoichiometric ratios thereof. However, the present disclosure is not limited thereto.

Referring to block 530, in some embodiments, the visualization includes a regional heat map associated with a respective conversion for a subset of wells 1100 in the array of wells of the first multi-well plate. For instance, in some embodiments, the subset of wells 1100 is selected based on a common reagent accommodated by each well 812 in the subset of wells, a common solvent accommodated by each well 812, a stoichiometric ratio associated with each 812, and/or the like. By way of example, referring to FIG. 11, a first subset of wells 1100-1 is associated with dimethylformamide (DMF) and triethylamine (Et₃N), and a second subset of wells 1100-2 is associated with methanol and Et₃N. Moreover, the first subset of wells 1100-1 is collectively determined (e.g., by model 160 of FIG. 2B) to satisfy a threshold conversion rate and the second subset of wells 1100-2 fails to satisfy the threshold conversion rate, a first color, shading, or transparency is applied to each well 812 in the first subset of wells 1100-1 (e.g., a transparent shading) and a second color, shading, or transparency is applied to each well 812 in the second subset of wells 1100-2, which allows for the visualization to graphical depict differences between DMF and methanol when reacting with Et₃N based on the regional color variation between the subsets of wells 1100.

Referring to block 532, in some embodiments, each well 812 in the subset of wells 1100 shares a contiguous boundary with at least one well in the subset of wells. For instance, in some embodiments, each well 812 in the subset of wells 1100 shares an edge and/or is physically adjacent to another well 812 in the subset of wells 1100, which allows for forming traceable patterns between wells 812 of the multi-well plate 800. Referring briefly to FIG. 8, a second well 812-2 shares a contiguous boundary with a third well 813-2 in the first subset of wells 1100-1, such that an edge portion of the second well 812-1 is adjacent to or touches an edge portion of the third well 812-2. In some embodiments, by sharing the contiguous boundary within the subset of wells 1100, the subset of wells 1100 are collectively associated through linking shared parameters of adjacent wells. For instance, in FIG. 8, the second well 812-2 and the third well 812-3 both utilize Et₃N as a reactant 1910 as a shared parameter. However, the present disclosure is not limited thereto.

Referring to block 534, in some embodiments, an exterior boundary of the subset of wells 1100 forms a closed-form polygon. As a non-limiting example, referring to FIG. 11, each subset of wells 1100 forms a rectangular polygon that accommodates 24 wells 812, which forms 16 subsets of wells 1100 for the multi-well plate 800. However, the present disclosure is not limited thereto. For instance, in some embodiments, the exterior boundary of the subset of wells 1100 forms an ellipsoid or circle, such as the second subset of wells 1100-2 of FIG. 9.

Referring to block 536-538, in some embodiments, the regional heat map includes an opacity gradient mapping for each corresponding well 812 in the array of wells of the first multi-well plate 800-1. In some embodiments, the opacity gradient mapping is based on a linear scale, a logarithmic scale, a rank scale, or an exponential scale.

Referring to block 540, in some embodiments, the method 500 includes assigning a first opacity of the opacity gradient mapping to the subset of wells 1100. In some embodiments, the method 500 includes assigning a second opacity of the opacity gradient mapping to the subset of wells 1100. For instance, in some embodiments, in accordance with a determination of a collective conversion of the subset of wells 1100 exceeds a fourth threshold conversion rate, a first opacity of 0% transparency is applied to the subset of wells 1100, and in accordance with a determination of the collective conversion of the subset of wells 1100 fails to exceed the fourth threshold conversion rate, a second opacity of 75% transparency is applied to the subset of wells 1100, indicating graphically through the visualization of the multi-well plate that the corresponding wells failed to satisfy a threshold condition.

Example Methods for Performing a Synthesis and/or Purification Task at a Molecular Foundry

As illustrated in FIGS. 5A-5C, another aspect of the present disclosure provides a method for performing a synthesis and/or purification task at a molecular foundry.

Referring to block 600 of FIG. 5A, in some embodiments, a method 600 for performing a synthesis and/or purification task at a molecular foundry (e.g., molecular foundry 200 of FIG. 1, molecular foundry 200 of FIG. 2C, molecular foundry 200 of method 300, etc.) is provided.

Referring to block 602, in some embodiments, the method 600 includes obtaining a first workflow (e.g., block 302 of FIG. 3A, workflow 2400 of FIG. 24, workflow 2400 of FIG. 25, workflow 2400 of FIG. 26, workflow 2400 of FIG. 27, user interfaces 1900 of FIGS. 8-28, etc.). In some embodiments, the first workflow 2400-1 includes a plurality of tasks for performing a first reaction, such as a plurality of synthesis tasks. As a non-limiting example, in some embodiments, the plurality of tasks includes a liquid chromatography task, an evaporation task, an agitation task, or a combination thereof, which allows for completing a wide variety of reactions at the molecular foundry 200.

Referring to block 604, in some embodiments, the plurality of tasks includes the liquid chromatography task, which provides for the separation and/or purification of the target compounds. In some embodiments, the liquid chromatography task includes one or more column location parameters, one or more solvent parameters, one or more mobile phase gradient parameters, one or more mass flow rate parameters, one or more stoichiometry parameters, one or more wavelength parameters, one or more epoch parameters, one or more volumetric parameters, one or more resolution parameters, one or more mass parameters, or a combination thereof.

Referring to block 606, in some embodiments, the plurality of tasks includes the evaporation task. Moreover, in some embodiments, the evaporation task includes one or more epoch parameters, one or more velocity parameters, one or more stoichiometry parameters, one or more temperature parameters, one or more frequency parameters, one or more pressure parameters, one or more human interaction parameters, or a combination thereof.

Referring to block 608, in some embodiments, the plurality of tasks includes the agitation task. Moreover, in some embodiments, the agitation task includes one or more temperature parameters, one or more power source parameters, one or more frequency parameters, one or more amplitude parameters, one or more epoch parameters, or a combination thereof.

Referring to block 610 of FIG. 5B, in some embodiments, the method 600 includes assigning a first selection of parameters for each task in the plurality of tasks. For instance, in some embodiments, the method 600 assigns a first frequency parameter (e.g., a first frequency of 120 Hertz (Hz)) when performing a first task and a second frequency parameter (e.g., a second frequency of 60 Hz) when performing a second task different from the first task. In some embodiments, the first selection of parameters is assigned in accordance with a determination of an amount of a target compound to be formed when executing a workflow 2400. As a non-limiting example, if the amount of the target compound was 100 grams of a salt, the first selection of parameters requires a first reactant of 38,750 mg of sodium added to a solution 2920. However, the present disclosure is not limited thereto.

Referring to block 612, in some embodiments, the first selection of parameters includes an amount of agitation. For instance, in some embodiments, the first selection of parameters include a frequency of the agitation, an amplitude of the agitation, a periodicity of the agitation, a velocity of the agitation, or a combination thereof. In some embodiments, the amount of agitation defines an internal flow condition of the solution 2920, such as whether the solution exhibits turbulent flow at a portion of the solution 2920. However, the present disclosure is not limited thereto.

Referring to block 614, in some embodiments, the first selection of parameters includes an amount of a reactant (e.g., blocks 352-360 of FIG. 3D, blocks 363-392 of FIG. 3F-3G, etc.), such as a mass of the reactant, a volume of the reactant, a concentration of the reactant, or the like.

Referring to block 616, in some embodiments, the first selection of parameters includes a reaction duration (e.g., blocks 352-360 of FIG. 3D, blocks 363-392 of FIG. 3F-3G, etc.).

Referring to block 618, in some embodiments, the first selection of parameters includes a reaction temperature (e.g., blocks 352-360 of FIG. 3D, blocks 363-392 of FIG. 3F-3G, reaction temperature 1710 of FIG. 17, etc.).

Referring to block 620, in some embodiments, the first selection of parameters includes a stoichiometric ratio between a first reactant and a second reactant (e.g., a first reactant and a solvent accommodated by the well 812).

Referring to block 626, in some embodiments, the method 600 includes executing the first workflow 2400-1 using the first selection of parameters. Accordingly, by executing the first workflow 2400-1 at the molecular foundry 200, a portion that is less than all of the plurality of synthesis tasks is deemed complete. For instance, in some embodiments, the executing the first workflow 2400-1 causes at least one synthesis task in the plurality of synthesis tasks to be deemed complete. In some such embodiments, the at least one synthesis task includes less than all of the plurality of synthesis tasks.

Referring to block 628 of FIG. 5C, in some embodiments, the method 600 includes determining a conversion efficiency of the first reaction in making a target compound or a purity of the target compound. In some embodiments, the conversion efficiency is determined concurrent and/or simultaneous to the executing of the first workflow, which allows for determining the conversion efficiency when some or all of the reaction occurs or immediately thereof. For instance, in some embodiments, a data set is obtained when performing the reaction and/or when the reaction is deemed complete (e.g., block 362 of FIG. 3D). In some such embodiments, the data set is applied to a model 160 that determines the conversion efficiency based on the data set, such as a first model 160-1 that is trained to determine the conversion efficiency of a first classification of solutions 2920. However, the present disclosure is not limited thereto.

Referring to block 630, in some embodiments, the method 600 includes assigning a second selection of parameters for one or more tasks in the plurality of tasks, such as by modifying a first parameter associated with a first task in the plurality of tasks, substituting the first parameter from the first task, adding a second parameter to the first task, or the like. For instance, in some embodiments, a model (e.g., model construct 160 of FIG. 2A) evaluations the conversion efficiency and identifiers one or more parameters for inclusion in the second selection of parameters that causes an increase in the conversion efficacy in comparison to a corresponding parameter in the first selection of parameters. As a non-limiting example, in some embodiments, the second selection of parameters includes an increase in at least one parameter in the first selection of parameters in accordance with a determination that the conversion efficiency of the first reaction or the purity of the target compound fails to satisfy a threshold conversion efficiency or purity. In some such embodiments, the second selection of parameters includes a decrease in at least one parameter in the first selection of parameters in accordance with a determination that the conversion efficiency of the first reaction or the purity of the target compound satisfies the threshold conversion efficiency or purity. Accordingly, by assigning the second selection of parameters for the one or more tasks, the method 600 allows for dynamically modifying performance of the workflow 2400 in order to ensure that an optimal or maximum conversion efficiency, rate, and/or value 154 is achieved by a particular reaction associated with the wells 812 of the multi-well plate 800.

As a non-limiting example, in some embodiments, the second selection of parameters modifies a reaction temperature 1710, substitutes a first solvent 1920-1 for a second solvent 1920-2, substitutes a first reagent 1910-1 for a second reagent 1920-1, modifies a wavelength of light illuminating a well 812, increases and/or decreases an intensity of light illuminating the well 812, increases and/or decreases a translational velocity of the solution 2920, increases and/or decreases a rotational velocity of the solution 2920, increases a temperature of the solution 2920, a modifies a resolution between a target peak and a surrounding peak of a spectral analysis, modifies a mass of the first solvent 1920-1, modifies a mass of the first reagent 1910-1, or a combination thereof.

Referring to block 632, in some embodiments, the threshold conversion efficiency or purity is a threshold conversion efficiency. Accordingly, in some such embodiments, the second selection of parameters is configured to improved conversion value for the first molecular reaction in comparison to if the workflow completed performing using only the first selection parameters without adapting based on information obtained when executing the workflow.

As a non-limiting example, in some embodiments, for a first apparatus (e.g., an evaporator apparatus of the molecular foundry 200), the instructions of the first selection for an evaporation task can be interpreted as:
Time (min): parameter 1, value 1 (e.g., 20 minute epoch duration)
Vortex (RPM): parameter 2, value 2 (e.g., 200 RPM)
Vortex acceleration: parameter 3, value 3 (e.g., 5 G)
Temperature (° C.): parameter 4, values 4-6 (e.g., values 4-6 is each 40° C.)
Pump mode: State 1, value 7 (e.g., ON State)
Pump pressure (mbar): parameter 5, value 8 (e.g., 14 mbar)
Pump frequency (Hz): parameter 7, value 9 (19 Hz)
   and the instructions for the second selection for the evaporation task can be interpreted as:
Time (min): parameter 1, value 1 (e.g., 20 minute epoch duration)
Vortex (RPM): parameter 2, value 10 (e.g., 400 RPM)
Vortex acceleration: parameter 3, value 3 (e.g., 5 G)
Temperature (C): parameter 4, values 4-6 (e.g., values 4-6 is each 40° C.)
Pump mode: State 1, value 5 (e.g., ON State)
Pump pressure (mbar): parameter 5, value 11 (e.g., 50 mbar)
Pump frequency (Hz): parameter 7, value 12 (35 Hz)

As another non-limiting example, in some embodiments, for a second apparatus (e.g., an agitation apparatus of the molecular foundry 200), the instructions of the first selection for an agitation task can be interpreted as:
Target temperature 1710 (° C.): parameter 1, value 1 (e.g., maintained at 75° C.)
Temperature offset (° C.): parameter 2, value 2 (e.g., 2° C. tolerance)
Frequency (RPM): parameter 3, value 3 (e.g., 400 RPM)
Amplitude (cm): parameter 4, value 4 (e.g., 2.2 cm)
Agitation mode: State 1, value 5 (e.g., ON State for 100 minutes)
Heater mode: State 1, value 6 (e.g., ON State for 90 minutes)
   and the instructions for the second selection for the agitation task can be interpreted as:
Target temperature 1710 (° C.): parameter 1, value 1 (e.g., maintained at 75° C.)
Temperature offset (° C.): parameter 2, value 1 (e.g., 1° C. tolerance)
Frequency (RPM): parameter 3, value 8 (e.g., 450 RPM)
Amplitude (cm): parameter 4, value 9 (e.g., 2.0 cm)
Agitation mode: State 1, value 6 (e.g., ON State for 900 minutes)
Heater mode: State 1, value 6 (e.g., ON State for 90 minutes)

Referring to block 634, in some embodiments, the conversion efficiency is between 20 percent and 80 percent. For instance, in some embodiments, the conversion efficiency is between 20 and 80 percent, 20 and 50 percent, 23 and 77 percent, 23 and 47 percent, 25 and 75 percent, 25 and 45 percent, 28 and 72 percent, 28 and 42 percent, 30 and 70 percent, 30 and 40 percent, 33 and 67 percent, 33 and 37 percent, 36 and 64 percent, 38 and 62 percent, 41 and 59 percent, 43 and 57 percent, 46 and 54 percent, 49 and 51 percent, 50 and 80 percent, 53 and 77 percent, 55 and 75 percent, 58 and 72 percent, 60 and 70 percent, or 63 and 67 percent.

In some embodiments, the conversion efficiency is at least 20 percent, at least 23 percent, at least 25 percent, at least 28 percent, at least 30 percent, at least 33 percent, at least 36 percent, at least 37 percent, at least 38 percent, at least 40 percent, at least 41 percent, at least 42 percent, at least 43 percent, at least 45 percent, at least 46 percent, at least 47 percent, at least 49 percent, at least 50 percent, at least 51 percent, at least 53 percent, at least 54 percent, at least 55 percent, at least 57 percent, at least 58 percent, at least 59 percent, at least 60 percent, at least 62 percent, at least 63 percent, at least 64 percent, at least 67 percent, at least 70 percent, at least 72 percent, at least 75 percent, at least 77 percent, or at least 80 percent.

In some embodiments, the conversion efficiency is at most 20 percent, at most 23 percent, at most 25 percent, at most 28 percent, at most 30 percent, at most 33 percent, at most 36 percent, at most 37 percent, at most 38 percent, at most 40 percent, at most 41 percent, at most 42 percent, at most 43 percent, at most 45 percent, at most 46 percent, at most 47 percent, at most 49 percent, at most 50 percent, at most 51 percent, at most 53 percent, at most 54 percent, at most 55 percent, at most 57 percent, at most 58 percent, at most 59 percent, at most 60 percent, at most 62 percent, at most 63 percent, at most 64 percent, at most 67 percent, at most 70 percent, at most 72 percent, at most 75 percent, at most 77 percent, or at most 80 percent.

Referring to block 636, in some embodiments, the threshold conversion efficiency or purity is a threshold purity. In some embodiments, the threshold purity is between 1 and 100 percent, 1 and 50 percent, 4 and 97 percent, 4 and 47 percent, 7 and 94 percent, 7 and 44 percent, 11 and 90 percent, 11 and 40 percent, 14 and 87 percent, 14 and 37 percent, 17 and 84 percent, 17 and 34 percent, 20 and 81 percent, 20 and 31 percent, 23 and 78 percent, 23 and 28 percent, 27 and 74 percent, 30 and 71 percent, 33 and 68 percent, 36 and 65 percent, 39 and 62 percent, 43 and 58 percent, 46 and 55 percent, 49 and 52 percent, 50 and 100 percent, 53 and 97 percent, 56 and 94 percent, 60 and 90 percent, 63 and 87 percent, 66 and 84 percent, 69 and 81 percent, or 72 and 78 percent. In some embodiments, the threshold purity is at least 1 percent, at least 4 percent, at least 7 percent, at least 11 percent, at least 14 percent, at least 17 percent, at least 20 percent, at least 23 percent, at least 27 percent, at least 28 percent, at least 30 percent, at least 31 percent, at least 33 percent, at least 34 percent, at least 36 percent, at least 37 percent, at least 39 percent, at least 40 percent, at least 43 percent, at least 44 percent, at least 46 percent, at least 47 percent, at least 49 percent, at least 50 percent, at least 52 percent, at least 53 percent, at least 55 percent, at least 56 percent, at least 58 percent, at least 60 percent, at least 62 percent, at least 63 percent, at least 65 percent, at least 66 percent, at least 68 percent, at least 69 percent, at least 71 percent, at least 72 percent, at least 74 percent, at least 78 percent, at least 81 percent, at least 84 percent, at least 87 percent, at least 90 percent, at least 94 percent, at least 97 percent, or at least 100 percent. In some embodiments, the threshold purity is at most 1 percent, at most 4 percent, at most 7 percent, at most 11 percent, at most 14 percent, at most 17 percent, at most 20 percent, at most 23 percent, at most 27 percent, at most 28 percent, at most 30 percent, at most 31 percent, at most 33 percent, at most 34 percent, at most 36 percent, at most 37 percent, at most 39 percent, at most 40 percent, at most 43 percent, at most 44 percent, at most 46 percent, at most 47 percent, at most 49 percent, at most 50 percent, at most 52 percent, at most 53 percent, at most 55 percent, at most 56 percent, at most 58 percent, at most 60 percent, at most 62 percent, at most 63 percent, at most 65 percent, at most 66 percent, at most 68 percent, at most 69 percent, at most 71 percent, at most 72 percent, at most 74 percent, at most 78 percent, at most 81 percent, at most 84 percent, at most 87 percent, at most 90 percent, at most 94 percent, at most 97 percent, or at most 100 percent.

Referring to block 638, in some embodiments, the method includes generating a second workflow 2400-2. In some embodiments, the second workflow 2400-2 includes some or all of the plurality of tasks for performing the first reaction, in which the second workflow 2400-2 includes the second selection of parameters. Accordingly, the method 600 allows for executing and evaluating the first workflow 2400-1 and generating an improved workflow in the form of the second workflow 2400-2 based on the evaluation of the first workflow 2400-1, such as before the first workflow 2400-1 is deemed complete at the molecular foundry. However, the present disclosure is not limited thereto.

Referring to block 640, in some embodiments, the method 600 includes executing the second workflow 2400-2, such as at the computing system 100 of the molecular foundry. In some embodiments, the executing of the second workflow 2400-2 ceases (e.g., terminates) execution of the first workflow 2400-1 at the molecular foundry 200. However, the present disclosure is not limited thereto. For instance, in some embodiments, the executing of the second workflow 2400-2 is performed concurrently, simultaneously, and/or after the first workflow 2400-1, which allows for comparing a complete performance of the first workflow 2400-1 against the second workflow 2400-2 and/or a third workflow 2400-3.

From this, the method 600 provides for performing the synthesis and/or purification task at the molecular foundry 200, such as through completing of some or all of the first workflow and/or the second workflow. Moreover, by executing the first workflow 2400-1, determining the conversion efficiency as the first workflow 2400-1 is being performed, and assigning the second selection of parameters based on the conversion efficiency allows for dynamically modifying the reactions for producing the target compounds in order to ensure that satisfactory conversion is achieved.

### Example Methods for Designing a Workflow

As illustrated in FIGS. 6A-6C, another aspect of the present disclosure provides a method for designing a workflow, such as a workflow for performing the synthesis of a compound using a molecular reaction. In some embodiments, the molecular reaction is a multistep molecular reaction.

Referring to block 700 of FIG. 6A, in some embodiments, a method 700 for designing a workflow is provided (e.g., workflow of block 302 of FIG. 3A, workflow of block 602 of FIG. 5A, etc.).

Referring to block 702, in some embodiments, the method 700 includes obtaining a selection of a first multi-well plate 800-1 in a plurality of multi-well plates 800. In some embodiments, each multi-well plate 800 in the plurality of multi-well plates 800 includes an array of wells 812. In some embodiments, the obtaining the selection of the first multi-well plate is performed as exemplified by at least block 362 of FIG. 3D. However, the present disclosure is not limited thereto.

Referring to block 704-708, in some embodiments, the method 700 includes assigning a corresponding identifier to each respective well 812 of the first multi-well plate 800-1. In some embodiments, the corresponding identifier includes an alphanumeric identifier associated with a first position in the array of wells 812. In some embodiments, the corresponding identifier includes a Cartesian identifier associated with a second position in the array of wells 812. In some embodiments, the corresponding identifier is as exemplified by the identifier(s) and/or coordinate(s) of blocks 386-390 of FIG. 3F.

Referring to block 710 of FIG. 6B, in some embodiments, the method 700 includes obtaining a plurality of user-defined conditions (e.g., conditions 142 of FIG. 2B), which define one or more parameters of a workflow for performing a reaction. For instance, referring briefly to FIG. 19, in some embodiments, the plurality of user-defined conditions 142 include a selection of one or more reactants for a reaction in a corresponding well 812 and one or more solvents for the reaction in the corresponding well 812. In some embodiments, the plurality of user-defined conditions includes a first concentration of the reactant 1910 and/or a second concentration of the solvent 1920. In some embodiments, the plurality of user-defined conditions includes a first equivalent of the reactant 1910 and a second equivalent of the solvent 1920 of the solution 2920. Accordingly, in some embodiments, the plurality of user-defined conditions advantageously allow an end-user to form subsets of wells 1100 based on groupings or patterns of user-defined conditions, such as reacting aldehydes for each well 812 in a first row of the multi-well plate 800 and reacting amines for each well 812 in a first column of the multi-well plate 800.

In some embodiments, the method 700 further includes obtaining a plurality of geometric conditions for each respective well 812 of the first multi-well plate 800-1. For instance, in some embodiments, the plurality of geometric conditions include an interior volume of the well 812, a material thickness of the well 812, a radius of curvature of the well 812, a diameter of the well 812, a hydraulic diameter of the well 812, an orientation of well 812, a dead-volume of the well 812, a free-volume of the well 812, or a combination thereof.

Referring to blocks 712-714, in some embodiments, the plurality of user-defined conditions includes one or more local reaction conditions associated with a first well 812-1 in the array of wells of the first multi-well plate, which allows for fine-grain control over the reaction in any one particular well 812 of the multi-well plate 800. By way of example, referring to FIG. 17, a first condition 142-1 is defined by the user such that for each respective well 812 associated with the multi-well plate 800, in accordance with a determination that a reaction temperature is less than 60° C., a red color is applied to the respective well 812, and, in accordance with a determination that a reaction temperature is greater than or equal to 60° C., no color is applied to the respective well 812. Accordingly, by including one or more local reaction conditions, the plurality of user-defined conditions allow for an end-user to configure how the workflow is executed at the molecular foundry on a well-by-well 812 basis.

In some embodiments, the plurality of user-defined conditions includes one or more regional reaction conditions associated with a subset of wells 1100 in the array of wells 812 of the first multi-well plate 800-1. By way of example, referring to FIG. 21, a third condition 142-3 is defined by the user such that for each respective well 812 associated with a first subset of columns (e.g., an upper portion of the columns, a first half of the columns, etc.) of the multi-well plate 800, in accordance with a determination that a threshold condition is exceeded, a first color is applied to the respective well 812, and, in accordance with a determination that the threshold condition is not satisfied, a second color is applied to the respective well 812. Accordingly, by including one or more regional reaction conditions, the plurality of user-defined conditions allow for the end-user to configure how the workflow is executed at the molecular foundry on a subset of wells 1100 basis.

Referring to block 716, in some embodiments, each well 812 in the subset of wells 1100 shares a contiguous boundary with at least one well 812 in the subset of wells 812. In some embodiments, the contiguous boundary is as exemplified by block 532 of FIG. 4C.

Referring to block 718, in some embodiments, the subset of wells 1100 includes a second well 812-2 that lacks a contiguous boundary with at least one well in the subset of wells 1100. For instance, referring to FIG. 8, the first well 812-1 is not adjacent to the third well 812-3 since the first well 812-1 and the third well 812-3 do not touch at respective edge portions. However, the present disclosure is not limited thereto.

Referring to block 720, in some embodiments, the one or more regional reaction conditions 142 includes a reagent-solvent pairing condition. For instance, in some embodiments, the one or more regional conditions 142 is configured to define a condition that is applied to each well 812 associated with a corresponding reagent-solvent pairing.

Referring to block 722, in some embodiments, the one or more regional reaction conditions 142 includes a reaction stoichiometry condition between a first reactant 1910-1 and a second reactant 1910-2. For instance, in some embodiments, the reaction stoichiometry condition defines a stoichiometric ratio between the first reactant 1910-1 and the second reactant 1910-2.

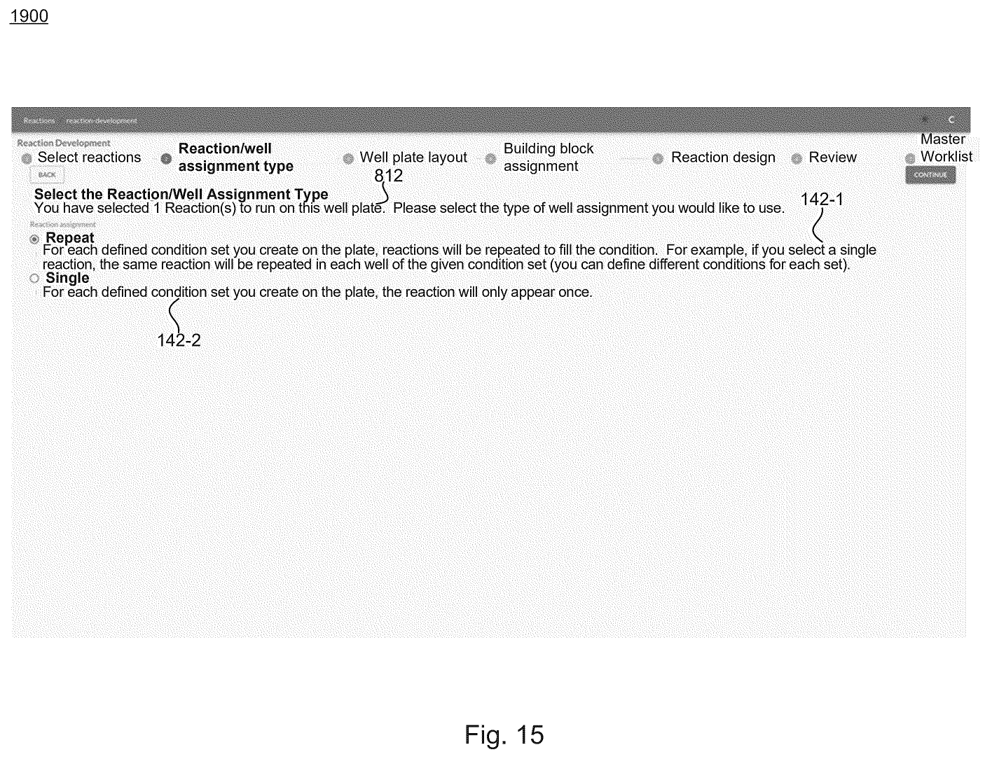

Referring to block 724 of FIG. 6C, in some embodiments, the plurality of user-defined conditions 142 includes one or more global reaction conditions associated with each well 812 in the array of wells 812 of the first multi-well plate 800-1, which allows the end-user to define a global condition 142 that is applied to the entirety of the first multi-well plate 800-1. For instance, referring briefly to FIG. 15, a first global condition 142-1 requires that, for each defined user-condition 142 associated with a well 812 and/or subset of wells 1100, a respective reaction will be repeated to fill the condition, whereas a second global condition 142-1 requires that, for each defined user-condition 142 associated with a well 812 and/or subset of wells 1100, the respective reaction will only appear once. In some such embodiments, the end-user is restricted from selecting the first global condition 142-1 or the second global condition 142-2, but not both the first and second global conditions 142. However, the present disclosure is not limited thereto.

Referring to block 726, in some embodiments, the one or more global reaction conditions 142 includes one or more epoch conditions, one or more temperature conditions, or a combination thereof. For instance, referring briefly to FIG. 17, the first global condition 142-1 defines a reaction temperature 1710 that is applied to each well 812 in the 130 wells 812 associated with a respective reaction for synthesis of one or more target compounds. However, the present disclosure is not limited thereto.

Referring to block 728, in some embodiments, the plurality of user-defined conditions 142 includes one or more reactants 19210 in one or more wells 812 in the array of wells of the first multi-well plate 800-1. For instance, in some embodiments, a respective user-defined condition 142 defines a first amount of a first reactant 1910-1 added to a first well 812-1 and/or a second amount of the first reactant 1910-1 different from the first amount added to a second well 812-2 different from the first well 812-1. In some embodiments, the respective user-defined condition 142 defines the first amount of the first reactant 1910-1 added to a first subset of wells 1100-1 and/or a second amount of a second reactant 1910-2 different from the first reactant added to a second subset of well 1100-2 different from the first subset of wells 1100-1.

Referring to block 730, in some embodiments, the plurality of user-defined conditions 142 defines a first order of operations associated with the first multi-well plate 800-1, a second order of operations associated with the subset of wells 1100, a third order of operations associated with each well 812 in the array of wells 812, or a combination thereof. For instance, in some embodiments, a first user-defined condition 142-1 defines a first task performed for each well 812 of the multi-well plate 800 (e.g., an agitation task that vibrations the multi-well plate 800), a second user-defined condition 142-2 defines a second task performed for a first subset of wells 1100 of the multi-well plate 800 that includes less than all of the wells 812 of the multi-well plate 800 (e.g., a reaction temperature task that applies a heat source locally to each well 812 in the first subset of wells 1100), and/or a third user-defined condition 142-3 defines a third task performed for a first well of the multi-well plate 800 (e.g., a liquid drawing task, block 3044 of FIG. 7E, etc.).

Referring to block 732, in some embodiments, the plurality of user-defined conditions 142 defines one or more Boolean conditions for visualizing a status of one or more wells 821 in the array of wells 812. For instance, referring briefly to FIG. 16, a first user-defined condition 142-1 defines a first threshold condition that applies a first color (e.g., a tan or peach color) to each well 812 that satisfies the first threshold condition, and a second user-defined condition 142-2 defines a second threshold condition that applies a second color (e.g., a red color) to each well 812 that satisfies the second threshold condition. For instance, in some embodiments, the first threshold condition is inclusion in a first subset of wells 1100 and/or having a first reactant and/or solvent pairing, and the second threshold condition is inclusion in a second subset of wells 110 and/or having a second reactant and/or solvent pairing. However, the present disclosure is not limited thereto. Referring briefly to FIG. 23, in some embodiments, the user-defined condition evaluates a reaction at one or more wells 812 in order to determine if one or more error conditions is satisfied and applies the second color if at least one well 812 in the one or more wells 812 satisfies a respective error condition in the one or more error conditions. By way of example, a first well 812-1 associated with an identifier 1730 “A1” satisfies a first error condition 142-1 by requiring one or more parameters of the reaction that causes an error preventing completion of the reaction at the corresponding well 812, such as by requesting addition of a compound that exceeds a volume of the well 812,

Referring to block 734, in some embodiments, the method 700 includes assigning a first indicia to the first well 812-1 in accordance with a determination the first well 812-1 in the array of wells satisfies a first Boolean condition 142-1. In some embodiments, the method 700 includes assigning a second indicia to the first well 812-2 in accordance with a determination the first well 812-2 in the array of wells fails to satisfy the first Boolean condition 142-1. For instance, in some embodiments, the method assigns a first color to the first well 812-1 in accordance with a determination the first well 812-1 in the array of wells satisfies a first Boolean condition 142-1. In some embodiments, the method 700 includes assigning a second color to the first well 812-2 in accordance with a determination the first well 812-2 in the array of wells fails to satisfy the first Boolean condition 142-1. As a non-limiting example, in some embodiments, the method assigns a first graphical icon to the first well 812-1 in accordance with a determination the first well 812-1 in the array of wells satisfies a second Boolean condition 142-1. In some embodiments, the method 700 includes assigning a second graphical icon to the first well 812-2 in accordance with a determination the first well 812-2 in the array of wells fails to satisfy the second Boolean condition 142-1. As yet another non-limiting example, in some embodiments, the method 700 assigns the first color to the first well 812-1 in accordance with a determination the first well 812-1 satisfies the first Boolean condition 142-1, assigns the second color to the first well 812-2 in accordance with a determination the first well 812-2 satisfies a second Boolean condition 142-2 and the first Boolean condition 142-1, assigns the first graphical icon to the first well 812-1 in accordance with a determination the first well 812-1 satisfies a third Boolean condition 142-1, and assigns the second graphical icon to the first well 812-2 in accordance with a determination the first well 812-2 in the array of wells fails to satisfy the third Boolean condition 142-1. However, the present disclosure in not limited thereto.

Referring to block 736, in some embodiments, the plurality of geometric conditions includes one or more dimensions associated with each respective well 812 in the array of wells, such as an opening diameter of the first well 812-1, a maximum diameter of the first well 812-1, a minimum diameter of the first well 812-1, a depth of the first well 812-1, a dead volume of the first well 812-1, a fluid volume of the first well 812-1, or the like. However, the present disclosure is not limited thereto.

Referring to block 738, in some embodiments, the method 700 includes generating, for display through a graphical user interface (e.g., user interface 78 of FIG. 2A, user interface 1900 of FIG. 8, user interface 1900 of FIG. 9, user interface 1900 of FIG. 11, user interface 1900 of FIG. 12, user interface 1900 of FIG. 13, user interface 1900 of FIG. 16, user interface 1900 of FIG. 17, user interface 1900 of FIG. 18, user interface 1900 of FIG. 19, user interface 1900 of FIG. 20, user interface 1900 of FIG. 21, user interface 1900 of FIG. 22, user interface 1900 of FIG. 23), a visualization of the first multi-well plate 800-1 based on the corresponding identifier assigned to each respective well 812 in the array of wells, the plurality of user-defined conditions 142, and/or the plurality of geometric conditions 142, which allows for an end-user to visualize the reaction conversion.

### Example Methods for Drawing a Liquid Solution

As illustrated in FIGS. 7A-7E, another aspect of the present disclosure provides a method for drawing a liquid solution (e.g., using pipettor 2910 of FIG. 29, etc.), such as during a workflow 2400 for performing the synthesis of a compound using a molecular reaction. In some embodiments, the molecular reaction is a multistep molecular reaction, which requires drawing and/or aspiring one or more fluids from and/or into the liquid solution.

Referring to block 3000 of FIG. 7A, a method 3000 for drawing a solution (e.g., solution 2920 of FIG. 29) into a robotic pipette tip (e.g., robotic pipette tip 2912 of pipettor 2910 of FIG. 20) is provided.

Referring to block 3002, in some embodiments, the method 300 includes obtaining a status of a solution 2920, such as the solution 2920 in a first well (e.g., well 812 of block 302 of FIG. 3A, well 812 of block 502 of FIG. 4A, well 812 of block 702 of FIG. 6A, etc.) in a multi-well container. In some embodiments, the status of the solution 2920 includes an identification of the solution 2920, such as an identification of one or more compounds of the solution 2920. For instance, in some embodiments, the identification of the solution 2920 is obtained from a configuration file 2400 and/or corresponding specification associated with a workflow 2400 that includes one or more tasks performed at the first well 812-1. However, the present disclosure is not limited thereto.

In some embodiments, the status of the first well 812-1 includes a dimension of the first well 812-1. For instance, in some embodiments, the dimension of the first well 812-1 includes an opening diameter of the first well 812-1, a maximum diameter of the first well 812-1, a minimum diameter of the first well 812-1, a depth of the first well 812-1, a dead volume of the first well 812-1, a fluid volume of the first well 812-1, or the like. However, the present disclosure is not limited thereto. Accordingly, by obtaining the dimension of the first well 812-1, the method 3000 (e.g., a model 160 of FIG. 2A) is informed about the spatial region consumed and/or utilizable by the first well 812-2.

Referring to block 3004, in some embodiments, the identification of the solution 2920 includes identifying a corresponding classification, in a plurality of classifications, of the solution 2920. For instance, in some embodiments, the solution 2920 and/or solvent 1920 is suitable for use in automation. In some embodiments, the solution 2920 and/or solvent 1920 has a boiling point, rate of evaporation, density, and/or surface tension that is the same or substantially the same or greater than that of water would be suitable for use in automation. In some embodiments, the solution 2920 and/or solvent 1920 has a boiling point, a rate of evaporation, density, and/or surface tension less than that of water that is not be ideal for use in automation. In some embodiments, the molecular reaction, and/or one or more instances thereof, is performed using a first classification associated with the solution 2920 and/or solvent 1920 suitable for use in automation, including but not limited to N-methyl-2-pyrrolidone (NMP), dimethylformamide (DMF), acetonitrile (MeCN), dimethyl sulfoxide (DMSO), or mixtures thereof). A non-limiting example of a solvent not ideal for use in automation is methylene chloride (DCM). In some embodiments, a solvent suitable for automation is a solvent capable of solubilizing one or more components of a reaction (e.g., reactants, reagents, catalysts) and/or exhibits thermal stability when heated during a reaction, including but not limited to N-methyl-2-pyrrolidone (NMP). However, the present disclosure is not limited thereto.

In some embodiments, the classification of the solution 2920 is associated with one or more classifications of pipetting velocities (e.g., one or more flow rates for drawing and/or aspirating a fluid from a well 812), one or more sample volume classifications, one or more leading air gap classifications, one or more trailing air gap classifications, one or more submerge depth classifications of the pipettor 2910, one or more delay classifications (e.g., one or more epochs defining time between when a drawing and/or aspirating begins and/or terminates), or the like.

Referring to block 3006, in some embodiments, the plurality of classifications is defined, at least in part, by a first source. For instance, in some embodiments, a supplier of the solution 2920 and/or a supplier of the pipettor 2910 provides a predetermined classification for use with the solution 2920. However, the present disclosure is not limited thereto. In some embodiments, the lookup table is obtained from the first source, which ensures accuracy and precision when evaluating reactions based on the corresponding classification of the solution 2920. However, the present disclosure is not limited thereto.

Referring to block 3008, in some embodiments, the plurality of classifications includes a first classification associated with one or more solvents 1920 that exceeds a first threshold conductivity and a first threshold volatility. For instance, in some embodiments, if the one or more solvents 1920 fail to exceed the first threshold volatility, then the one or more solvents 1920 have a tendency to drip from an opening of a robotic pipette tip 2912 due, such as due to high vapor pressure that forms inside a cavity at the opening of the robotic pipette tip 2912. Moreover, when the one or more solvents 1920 exceed the first threshold conductivity, the one or more solvents can utilize conductive liquid level detection processes in order to determine a liquid volume and/or depth of the solution 2920 within the well 812. However, the present disclosure is not limited thereto.

Referring to block 3010, in some embodiments, the one or more solvents 1920 of the first classification includes N-methyl-2-pyrrolidone (NMP), dimethyl sulfoxide (DMSO), water, acetic acid (AcOH), or a combination thereof.

Referring to block 3012, in some embodiments, the plurality of classifications includes a second classification associated with one or more solvents 1920 that exceeds a second threshold conductivity and fails to exceed a second threshold volatility.

Referring to block 3014 of FIG. 7B, in some embodiments, the one or more solvents 1920 of the second classification includes methanol.

Referring to block 3016, in some embodiments, the plurality of classifications includes a third classification associated with one or more solvents 1920 that fails to exceed a third threshold conductivity and exceeds a third threshold volatility.

Referring to block 3018, in some embodiments, the plurality of classifications includes a fourth classification associated with one or more solvents 1920 that fails to exceed a fourth threshold conductivity and a fourth threshold volatility.

Referring to block 3020, in some embodiments, the one or more solvents 1920 of the fourth classification comprises N,N-diisopropylethylamine (DIPEA)

Referring to block 3022, in some embodiments, the solution 2920 includes (i) a first component solution selected from the first classification or the second classification and (ii) a second component solution selected from the third classification or the fourth classification.

Referring to block 3024 of FIG. 7C, in some embodiments, the solution include (i) a first component solution selected from the first classification or the second classification and (ii) a second component solution selected from the third classification or the fourth classification. In some embodiments, the solution 2920 exceeds the first threshold conductivity of the first classification or the second threshold conductivity of the second classification.

Referring to block 3026, in some embodiments, the solution 2920 includes (i) a first component solution selected from the first classification or the second classification and (ii) a second component solution selected from the third classification or the fourth classification. In some embodiments, the solution 2920 exceeds the first threshold conductivity of the first classification and the second threshold conductivity of the second classification.

Referring to block 3028, in some embodiments, the status of the first well 812-1 further includes a depth of the solution 2920, such as a distance from a lower end portion of an interior surface of the first well 812-1 to an upper end surface of the solution 2920. For instance, referring briefly to FIG. 29, in some embodiments, the depth is a distance L1 from the lower end portion of the first well 812-1 to the meniscus formed by the solution 2920 at the interior surface of the first well 812-1. However, the present disclosure is not limited thereto. Accordingly, by obtaining the depth of the solution 2920, the method 3000 is capable of identifying a spatial region consumed by the solution 2920 within the first well 812-1 allowing for precise drawing of the solution 2920 from the first well 812-1.

Referring to block 3030, in some embodiments, the status of the first well 812-1 further includes a dead volume of the solution 2920, such as a first volume of the solution 2920 at the lower end portion of the interior surface of the first well 812-1. In some embodiments, the dead volume of the solution 2029 is the amount of liquid remaining in the first well 812-1 after drawing liquid from the first well 812-2, such as a volume of the well 812-1 associated with a region from the lower end portion of the well 812 to a lower end portion of the pipettor 2910.

Referring to block 3032, in some embodiments, the status of the first well 812-1 further includes a depth of the lower end portion of the first well 812-1. For instance, referring to FIG. 29, in some embodiments, the depth of the lower end portion of the first well 812-1 is a distance L4 from an upper edge portion of the first well 812-1 to the lower end portion of the first well 812-1.

Referring to block 3034, in some embodiments, the dimension of the first well 812-1 includes an interior volume of the first well 812-1, such as an internal volume of the first well 812-1 based on diameter D1 of FIG. 29.

Referring to block 3034, in some embodiments, the method 3000 includes obtaining, using a lookup table, a conductivity of the solution 2920. Accordingly, by obtaining the conductivity of the solution 2920, the method 3000 determines a volume of the solution 2920 accommodated by the first well 812-1 based on the conductivity of the solution 2920 and the dimension of the first well 812-2.

Referring to block 3036 of FIG. 7D, in some embodiments, the obtaining further includes using the lookup table to obtain a volatility of the solution 2920. For instance, in some embodiments, the volatility of the solution 2920 is obtained using a conductivity-to-volatility data structure associated with the lookup table. However, the present disclosure is not limited thereto.

Referring to block 3038, in some embodiments, in accordance with a determination that the volume of the solution 2920 exceeds a third threshold value, the at least one traverse instruction includes traversing the lower end portion of the robotic pipette tip 2912. In some embodiments, in accordance with a determination that the volume of the solution 2920 fails to exceed the third threshold value, the at least one traverse instruction includes traversing the lower end portion of the first well 812-1 in the first vertical direction, such as by lowering the pipettor 2910 deeper into the solution 2920 towards the lower end portion of the first well 812-1.

Referring to block 3040, in some embodiments, the obtaining further includes conducting the generating in accordance with a determination the volume of the solution 2920 satisfies a fourth threshold value based on the interior volume of the first well 812-1. In some embodiments, the obtaining further includes ceasing the performing the one or more execution instructions at the liquid handling robot in accordance with a determination the volume of the solution 2920 fails to exceed the fourth threshold value.

Referring to block 3042, in some embodiments, the method 3000 includes generating, in accordance with the volume of the solution 2920 and the dimension of the first well 812-1, one or more execution instructions for drawing some or all of the solution 2920 from the first well using the robotic pipette tip 2912. In some embodiments, the one or more execution instructions include an opening at a lower end portion of the robotic pipette tip 2912. In some embodiments, the one or more execution instructions comprises at least one traverse instruction to traverse either the lower end portion of the robotic pipette tip or a lower end portion of the first well in a first vertical direction. In some embodiments, the at least one traverse instruction comprises a coordinate instruction and a velocity instruction.

Referring to block 3044 of FIG. 7E, in some embodiments, the method 3000 includes performing the one or more execution instructions at a liquid handling robot at a molecular foundry 200 coupled to the robotic pipette tip, which draws the solution into the robotic pipette tip.

Referring to block 3046, in some embodiments, the method 3000 includes determining, concurrent with the performing the one or more execution instructions at the liquid handling robot, a capacitance of the solution. In some embodiments, in accordance with a determination the capacity of the solution exceeds a first threshold value, the method includes continuing with the performing the one or more execution instructions at the liquid handling robot. In some embodiments, in accordance with a determination the capacitance of the solution fails to exceed the first threshold value, the method includes ceasing the performing the one or more execution instructions at the liquid handling robot.

Referring to block 3048, in some embodiments, the method 3000 includes determining, concurrent with the performing the one or more execution instructions at the liquid handling robot (e.g., block 3044 of FIG. 7E), the depth of the lower end portion of the first well 812-1.

Another aspect of the present disclosure includes a system, including a memory; one or more processors; and one or more modules stored in the memory and configured for execution by the one or more processors, the one or more modules including instructions for performing any of the methods disclosed above.

Another aspect of the present disclosure includes a non-transitory computer readable storage medium, the non-transitory computer readable storage medium storing one or more programs for execution by one or more processors of a computer system, the one or more computer programs including instructions for performing any of the methods disclosed above.

In some embodiments, the systems and methods disclosed herein are advantageously used in any number of applications, including but not limited to hit discovery, hit-to-lead discovery, lead optimization, off-target side-effect prediction, molecular dynamics simulations, toxicity prediction, potency optimization, selectivity optimization, fitness modeling, drug repurposing, drug resistance prediction, personalized medicine, drug trial design, agrochemical design, and/or materials science.

### EXAMPLES

### Example 1—Improved Molecule Design for Drug Discovery Using Machine Learning Models

Molecular reactions conventionally used in drug discovery are performed by traditional chemistry methods. However, the use of a limited set of molecular reactions has led to a narrowly populated chemical space. In particular, repeated chemical synthesis efforts using similar chemistry and similar molecules does not lead to a greater number of drug candidates; while approximately 100,000,000 molecules have been synthesized in human history, the rate of drug approval has remained relatively constant.

To solve multiparameter problems, such as the discovery of drug-like molecules having properties that will function in vivo, the presently disclosed systems and methods aim to explore new types of molecules in a different chemical space. For instance, in some embodiments, predicted properties for a set of candidate molecules is obtained using machine learning approaches, in accordance with some embodiments of the present disclosure. Compared with Enamine, a widely used, conventional virtual library, the candidate molecules generated using the presently disclosed machine learning approaches were predicted to exhibit higher target inhibition and higher ADME scores.

Automated chemistry has the power to learn new molecular reactions using multiple reaction conditions. Furthermore, the development of new chemistry can lead to novel building blocks and new small molecules for use in the design and development of drug candidates that improve upon traditional methods.

### Buchwald Cross Coupling Reaction

A non-limiting example of a reaction suitable for automated reaction development is the Buchwald cross coupling reaction. Generally, the Buchwald cross coupling reaction is the reaction between an aryl halide and an amine or amide to form a new aryl C—N bond using a palladium catalyst, ligand, and base. Scheme 1 illustrates a non-limiting example of a general synthetic scheme of the Buchwald cross coupling.

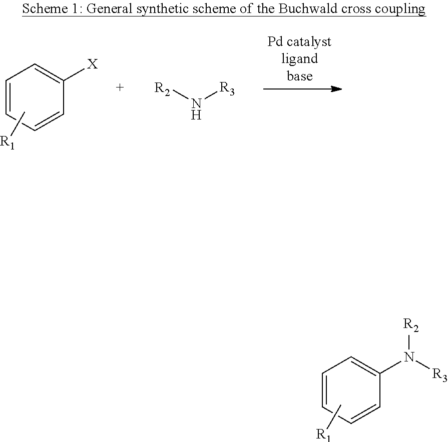

### Reactants

In this Example, for the exploratory and optimization phases of reaction development, six of one reactant and four of another reactant are used to probe the reactivity of desired conditions, and the reactants encompass the reactivity that is being tested (i.e., Buchwald cross coupling). In this case, initial, general reaction conditions for an automated synthesis for the Buchwald cross coupling were examined. The study sought to identify general reaction conditions, including identify a broad variety of reactants capable of carrying out the reaction, and within the 6×4 reagent constraints. The study also included identifying building blocks capable of being identified using liquid chromatography/mass spectroscopy (LCMS) for analysis and determination of percent conversion of the reaction. Desirable building blocks have a molecular weight (MW) of 150 g/mol or greater, are capable of being ionized by electrospray ionization (ESI), and are UV active. Additionally, availability and cost of the reactant are also factor that can be considered in reactant selection.

### Aryl Halides

In this Example, aryl halides are the set of six reactants. Non-limiting examples of factors considered in selecting the aryl halides include the identity of the halide or pseudohalide, the sterics surrounding the halide, and the electronics of the ring. As bromides and chlorides are more common and commercially available than iodides and triflates, two examples of bromides and chlorides were used. Scheme 2 below shows the structures of the six selected aryl halides.

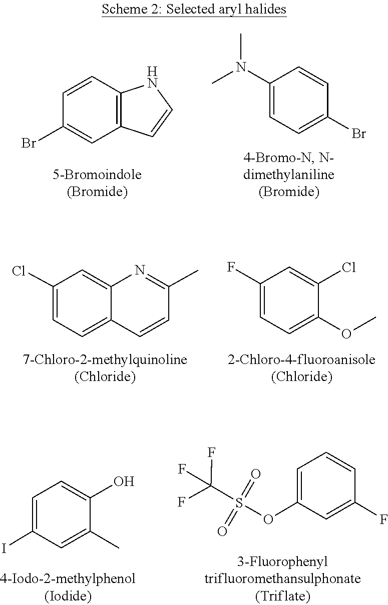

### Amines

In this Example, amines are the set of four reactants. Non-limiting examples of factors considered in selecting the amines include whether the nitrogen is in an amine or an amide, whether the amine is a primary or secondary amine, or whether the amine is an aryl or alkyl substituted amine. The four selected amines are shown in Scheme 3 below. By varying the structures and electronics of the aryl halides and amines, the selected six aryl halides and four amines provide a broad range of reactants for exploring conditions for the automated Buchwald cross coupling.

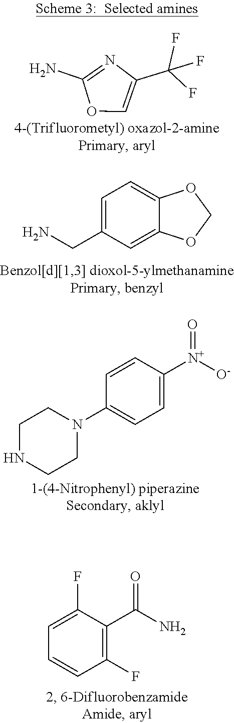 [SMILES: Nc1nc(C(F)(F)F)co1 ; NCc1ccc2c(c1)OCO2 ; O=[N+]([O-])c1ccc(N2CCNCC2)cc1 ; NC(=O)c1c(F)cccc1F]

### Amidation

A non-limiting example of a reaction suitable for automated reaction development is an amidation reaction. Scheme 4 illustrates a non-limiting example of a general synthetic scheme of an amidation reaction.

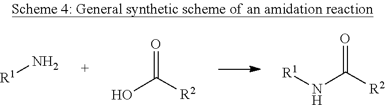

In this Example, six amines and four carboxylic acids are selected as reactants to form a set of 6×4 reactants (see, Schemes 5 and 6 for structures of amines and carboxylic acids). Four different solvents are selected for examination (e.g., N-methyl-2-pyrrolidone (NMP), dimethylformamide (DMF), acetonitrile (MeCN), and dimethyl sulfoxide (DMSO)), thereby providing 96 possible combinations of reactants and solvent for evaluation. The total number of combinations of reactions can be further expanded by treating each of the specific combinations of reactants and solvents with different sets of reagents (e.g., coupling agents, bases, acids, etc.) and under different reaction conditions.

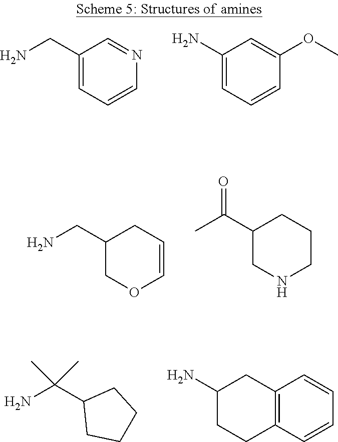

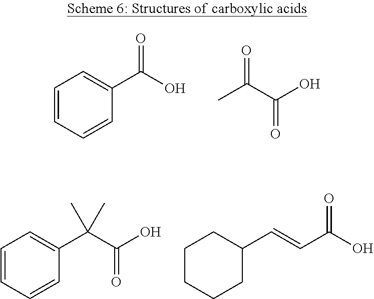

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method for implementing a workflow, the method comprising:
A) obtaining an initial workflow, the initial workflow comprising:
a selection of a plurality of target compounds, and
a plurality of synthesis tasks collectively configured to synthesize each target compound in the plurality of target compounds, wherein each synthesis task in the plurality of synthesis tasks includes a corresponding specification comprising (i) an identification of respective solvent in one or more solvents, (ii) an amount of at least one reactant in a plurality of reactants, (iii) an x-y address of a well in a first multi-well plate comprising a plurality of wells, (iv) a reaction duration, (v) a reaction temperature, and (vi) a reaction volume;
B) performing at least a respective subset of the plurality of synthesis tasks of the initial workflow, at a molecular foundry that comprises a plate handler for the first multi-well plate and one or more liquid handlers for at least the plurality of reactants, wherein the performing B) comprises reacting the plurality of reactants in the plurality of wells in accordance with the corresponding specification of at least the respective subset of the plurality of synthesis tasks;
C) informing a first data set associated with a physical property of a liquid in each well specified by the corresponding specification of each synthesis task in at least the respective subset set of synthesis tasks;
D) repeating the performing B) and obtaining C) until each synthesis task in the plurality of synthesis tasks has been performed;
E) determining an amount of each target compound in the plurality of target compounds that was synthesized in accordance with the plurality of synthesis tasks using the first data set; and
F) using the amount of each target compound in the plurality of target compounds that was synthesized to amend the corresponding specification of each synthesis task in the plurality of synthesis tasks.

2. The method of claim 1, wherein the using F) comprises training a model that estimates target compound synthetic efficiency as a function of synthesis task specification, wherein the model comprises a plurality of parameters, through application of a training procedure comprising:
i) applying the synthesis task specification of one or more synthesis tasks in the plurality of synthesis tasks for the synthesis of a corresponding target compound in the plurality of target compounds thereby obtaining a calculated synthetic efficiency for the corresponding target compound;
ii) determining a difference between (a) an efficiency of the corresponding target compound as determined by the model and (b) an actual efficiency of the corresponding target compound as determined by the first data set; and
iii) back-propagating the difference between the efficiency and the actual efficiency through the plurality of the models thereby training the model.

3. The method of claim 2, wherein the training procedure is repeated for each target compound, or batch of target compounds, in the plurality of target compounds.

4. The method of claim 1, wherein the plurality of synthesis tasks encodes a plurality of different specifications for synthesizing a first target compound in the plurality of target compounds and the using F) comprises pruning out a first subset of the plurality of different specifications for synthesizing the first target compound from the initial synthesis tasks that the first data set indicates synthesized the first target compound at a lower efficiency than a second subset of the plurality of different specifications for synthesizing the first target compound.

5. The method according to any preceding claim, wherein the physical property is determined using spectroscopy.

6. The method of claim 5, wherein the spectroscopy is ultraviolet (UV) spectroscopy and the physical property is absorbance of UV light.

7. The method of claim 5, wherein the spectroscopy is light spectroscopy and the physical property is absorbance of visible light.

8. The method of claim 5, wherein the spectroscopy is infrared (IR) spectroscopy and the physical property is absorbance of IR light.

9. The method of claim 5, wherein the spectroscopy is atomic absorption spectroscopy and the physical property is absorbance of light.

10. The method of claim 5, wherein the spectroscopy is inductively coupled plasma optical emission spectroscopy (ICP-OES) and the physical property is light emission.

11. The method of claim 5, wherein the spectroscopy is fluorescence spectroscopy and the physical property is light emission.

12. The method of claim 5, wherein the spectroscopy is Raman spectroscopy and the physical property is vibrational or rotational model of atoms of the target compound.

13. The method according to any preceding claim, wherein the initial workflow further comprises one or more plating tasks, one or more filtration tasks, one or more dilution tasks, one or more analytical tasks, or any combination thereof.

14. The method according to any preceding claim, wherein the plurality of target compounds consists of organic compounds.

15. The method according to any preceding claim, wherein the performing B) and/or the informing C) is conducted without human intervention.

16. The method according to any preceding claim, wherein the obtaining B) further comprises illuminating a field of view across the first multi-well plate with substantially uniform optical characteristics across the field of view.

17. The method of claim 16, wherein a spectral range of light when illuminating the field of view is between 250 nanometers (nm) and 315 nm.

18. The method according to claim 16, wherein the first data set comprises a first plurality of data elements associated with the field of view prior to the illuminating and a second plurality of data elements associated with the field of view when illuminated the informing C).

19. A method for visualizing a reaction conversion, the method comprising:
A) obtaining a selection of a first multi-well plate in a plurality of multi-well plates, wherein each multi-well plate in the plurality of multi-well plates comprises an array of wells;
B) assigning a corresponding identifier to each respective well of the first multi-well plate, wherein the corresponding identifier is associated with (i) a corresponding reagent-solvent pairing accommodated by the respective well and (ii) a corresponding reaction stoichiometry;
C) evaluating, when performing a reaction at a molecular foundry, a conversion for each respective well of the first multi-well plate, thereby producing a conversion data set; and
D) generating, for display through a graphical user interface, a visualization of the first multi-well plate based on the conversion data set, thereby visualizing the reaction conversion.

20. A method for performing a synthesis and/or purification task at a molecular foundry, the method comprising:
A) obtaining a first workflow comprising a plurality of tasks for performing a first reaction, wherein the plurality of tasks comprises a liquid chromatography task, an evaporation task, an agitation task, or a combination thereof;
B) assigning a first selection of parameters for each task in the plurality of tasks;
C) executing the first workflow using the first selection of parameters;
D) determining, concurrent with the executing C), (i) a conversion efficiency of the first reaction in making a target compound or (ii) a purity of the target compound;
E) assigning a second selection of parameters for one or more tasks in the plurality of tasks, wherein
the second selection of parameters comprises an increase in at least one parameter in the first selection of parameters in accordance with a determination that the conversion efficiency of the first reaction or the purity of the target compound fails to satisfy a threshold conversion efficiency or purity, and
the second selection of parameters comprises a decrease in at least one parameter in the first selection of parameters in accordance with a determination that the conversion efficiency of the first reaction or the purity of the target compound satisfies the threshold conversion efficiency or purity; and
F) generating a second workflow comprising some or all of the plurality of tasks for performing the first reaction, wherein the second workflow comprises the second selection of parameters; and
G) executing the second workflow, thereby performing the synthesis and/or purification task at the molecular foundry.
